(19) United States
(12) Patent Application Publication
(10) Pub. No.: US 2013/0018039 A1
(43) Pub. Date: Jan. 17, 2013

(54) IMIDAZOLYL-IMIDAZOLES AS KINASE INHIBITORS

(71) Applicants: Vera Q. Bodmer, Collegeville, PA (US); Linda N. Casillas, Collegeville, PA (US); Michael P. DeMartino, Collegeville, PA (US); Philip A. Harris, Collegeville, PA (US); Bryan W. King, Collegeville, PA (US); Ami Lakdawala Shah, King of Prussia, PA (US); Lara Kathryn Leister, Collegeville, PA (US); Joshi M. Ramanjulu, Collegeville, PA (US); Joseph J. Romano, Collegeville, PA (US); Gren Z. Wang, Collegeville, PA (US); Matthew A. Wilson, Moreland Hills, OH (US); David Duff Wisnoski, Collegeville, PA (US)

(72) Inventors: Vera Q. Bodmer, Collegeville, PA (US); Linda N. Casillas, Collegeville, PA (US); Michael P. DeMartino, Collegeville, PA (US); Philip A. Harris, Collegeville, PA (US); Bryan W. King, Collegeville, PA (US); Ami Lakdawala Shah, King of Prussia, PA (US); Lara Kathryn Leister, Collegeville, PA (US); Joshi M. Ramanjulu, Collegeville, PA (US); Joseph J. Romano, Collegeville, PA (US); Gren Z. Wang, Collegeville, PA (US); Matthew A. Wilson, Moreland Hills, OH (US); David Duff Wisnoski, Collegeville, PA (US)

(21) Appl. No.: 13/637,443

(22) PCT Filed: Mar. 31, 2011
(86) PCT No.: PCT/US11/30677

§ 371 Date: Sep. 26, 2012

Related U.S. Application Data
(60) Provisional application No. 61/319,572, filed on Mar. 31, 2010.

(51) Int. Cl. A61K 31/4184; C07D 401/14; C07D 405/14; C07D 235/14; C07D 417/14; C07D 403/14; C07D 471/04; A61K 31/5377; A61K 31/4439; A61K 31/428; A61K 31/4245; A61K 31/437; A61K 31/496; A61P 17/00; A61P 3/10; A61P 19/02; A61P 1/04; A61P 1/16; A61P 11/06; A61P 37/06; C07D 413/14

(52) U.S. Cl. 514/210.21; 544/139; 546/273.4; 548/306.1; 548/159; 548/305.1; 548/126; 546/118; 544/135; 544/127; 514/234.5; 514/338; 514/394; 514/367; 514/362; 514/303; 514/254.02; 514/234.2

(57) ABSTRACT

Disclosed are compounds having the formula: wherein R^{1A}, R^{1B}, R² and R³ are defined herein, and methods of making and using the same.

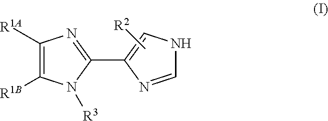

### BACKGROUND OF THE INVENTION

1. Field of the Invention

The present invention relates to imidazolyl-imidazoles that inhibit RIP2 kinase and methods of making and using the same. Specifically, the present invention relates to substituted benzimidazoles as RIP2 kinase inhibitors.

2. Background of the Invention

Receptor interacting protein-2 (RIP2) kinase, which is also referred to as CARD3, RICK, CARDIAK, or RIPK2, is a TKL family serine/threonine protein kinase involved in innate immune signaling. RIP2 kinase is composed of an N-terminal kinase domain and a C-terminal caspase-recruitment domain (CARD) linked via an intermediate (IM) region ((1998) J. Biol. Chem. 273, 12296-12300; (1998) Current Biology 8, 885-889; and (1998) J. Biol. Chem. 273, 16968-16975). The CARD domain of RIP2 kinase mediates interaction with other CARD-containing proteins, such as NOD1 and NOD2 ((2000) J. Biol. Chem. 275, 27823-27831 and (2001) EMBO reports 2, 736-742). NOD1 and NOD2 are cytoplasmic receptors which play a key role in innate immune surveillance. They recognize both gram positive and gram negative bacterial pathogens and are activated by specific peptidoglycan motifs, diaminopimelic acid (i.e., DAP) and muramyl dipeptide (MDP), respectively ((2007) J Immunol 178, 2380-2386).

Following activation, RIP2 kinase associates with NOD1 or NOD2 and appears to function principally as a molecular scaffold to bring together other kinases (TAK1, IKKα/β/γ) involved in NF-κB and mitogen-activated protein kinase activation ((2006) Nature Reviews Immunology 6, 9-20). RIP2 kinase undergoes a K63-linked polyubiquitination on lysine-209 which facilitates TAK1 recruitment ((2008) EMBO Journal 27, 373-383). This post-translational modification is required for signaling as mutation of this residue prevents NOD1/2 mediated NF-kB activation. RIP2 kinase also undergoes autophosphorylation on serine-176, and possibly other residues ((2006) Cellular Signalling 18, 2223-2229). Studies using kinase dead mutants (K47A) and non-selective small molecule inhibitors have demonstrated that RIP2 kinase activity is important for regulating the stability of RIP2 kinase expression and signaling ((2007) Biochem J 404, 179-190 and (2009) J. Biol. Chem. 284, 19183-19188).

Dysregulation of RIP2-dependent signaling has been linked to autoinflammatory diseases. Gain-of-function mutations in the NACHT-domain of NOD2 cause Blau Syndrome/Early-onset Sarcoidosis, a pediatric granulomateous disease characterized by uveitis, dermatitis, and arthritis ((2001) Nature Genetics 29, 19-20; (2005) Journal of Rheumatology 32, 373-375; (2005) Current Rheumatology Reports 7, 427-433; (2005) Blood 105, 1195-1197; (2005) European Journal of Human Genetics 13, 742-747; (2006) American Journal of Ophthalmology 142, 1089-1092; (2006) Arthritis & Rheumatism 54, 3337-3344; (2009) Arthritis & Rheumatism 60, 1797-1803; and (2010) Rheumatology 49, 194-196). Mutations in the LRR-domain of NOD2 have been strongly linked to susceptibility to Crohn's Disease ((2002) Am. J. Hum. Genet. 70, 845-857; (2004) European Journal of Human Genetics 12, 206-212; (2008) Mucosal Immunology (2008) 1 (Suppl 1), S5-S9. 1, S5-S9; (2008) Inflammatory Bowel Diseases 14, 295-302; (2008) Experimental Dermatology 17, 1057-1058; (2008) British Medical Bulletin 87, 17-30; (2009) Inflammatory Bowel Diseases 15, 1145-1154 and (2009) Microbes and Infection 11, 912-918). Mutations in NOD1 have been associated with asthma ((2005) Hum. Mol. Genet. 14, 935-941) and early-onset and extra-intestinal inflammatory bowel disease ((2005) Hum. Mol. Genet. 14, 1245-1250). Genetic and functional studies have also suggested a role for RIP2-dependent signaling in a variety of other granulomateous disorders, such as sarcoidosis ((2009) Journal of Clinical Immunology 29, 78-89 and (2006) Sarcoidosis Vasculitis and Diffuse Lung Diseases 23, 23-29) and Wegner's Granulomatosis ((2009) Diagnostic Pathology 4, 23).

A potent, selective, small molecule inhibitor of RIP2 kinase activity would block RIP2-dependent pro-inflammatory signaling and thereby provide a therapeutic benefit in autoinflammatory diseases characterized in increased and/or dysregulated RIP2 kinase activity.

### SUMMARY OF THE INVENTION

The invention is directed imidazolyl-imidazoles. Specifically, the invention is directed to a compound according to Formula (I):

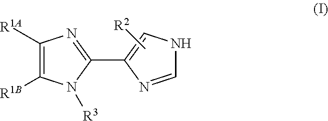

wherein:

R^{1A} and R^{1B} are each independently selected from H and a (C₁-C₆)alkyl group,

or R^{1A} and R^{1B}, taken together with the atoms through which they are attached, form a 6-membered non-aromatic carbocyclic ring or an optionally substituted 6-membered aromatic carbocyclic or heterocyclic ring,

wherein the 6-membered aromatic heterocyclic ring contains one or two nitrogen heteroatoms, and the 6-membered aromatic carbocyclic or heterocyclic ring is optionally substituted by 1-3 substituents each independently selected from halogen, cyano, (C₁-C₄)alkyl, halo(C₁-C₆)alkyl, hydroxy(C₁-C₆)alkyl, (C₁-C₄)alkoxy, halo(C₁-C₆)alkoxy, (C₁-C₄ alkyl)amino-, hydroxy(C₂-C₄ alkyl)amino-, (C₁-C₆ alkyl)(C₁-C₄ alkyl)amino-, (hydroxy(C₂-C₄ alkyl))(C₁-C₄ alkyl)amino-, —CO₂H, —CO₂(C₁-C₄alkyl), —CONH₂, —CONH(C₁-C₄alkyl), —CON(C₁-C₄alkyl)(C₁-C₆alkyl), —CONH(aryl), —CONH(heteroaryl), —SO₂NH₂, —SO₂NH(C₁-C₄alkyl), —SO₂NH(—C₁-C₄alkyl-phenyl), —SO₂N(C₁-C₄alkyl)(C₁-C₆alkyl), heterocycloalkyl, —CO(heterocycloalkyl), and —SO₂(heterocycloalkyl),

wherein any of said heterocycloalkyl is optionally substituted by 1-3 substituents each independently selected from hydroxy, (C₁-C₆)alkyl and hydroxy(C₁-C₄)alkyl,

any of said (C₁-C₄alkyl) is optionally substituted by 1-3 substituents each independently selected from (C₁-C₆)alkoxy, heterocycloalkyl, amino, (C₁-C₄ alkyl)amino-, and (C₁-C₄ alkyl)(C₁-C₄ alkyl)amino-, and

said aryl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy;

R² is monocyclic or bicyclic aryl or monocyclic or bicyclic heteroaryl, optionally substituted by one, two or three R^{2A} substituents,

wherein each R^{2A} is independently selected from halogen, cyano, (C₁-C₄)alkyl, halo(C₁-C₄)alkyl, C₁-C₄ alkoxy, hydroxyl, —CO₂H, —CO₂(C₁-C₄)alkyl, —CONH₂, —CONH(C₁-C₄alkyl), —CON(C₁-C₄alkyl)(C₁-C₄alkyl), phenylC₁-C₄ alkoxy, C₁-C₄ alkylthio-, —SO₂(C₁-C₄)alkyl, —SO₂NH₂, —SO₂NH(C₁-C₄alkyl), —SO₂N(C₁-C₄alkyl)(C₁-C₄alkyl), and monocyclic or bicyclic heteroaryl optionally substituted by (C₁-C₄)alkyl;

R³ is (C₁-C₄)alkyl, (C₁-C₂)alkoxy(C₁-C₄)alkyl-, hydroxy(C₂-C₄)alkyl-, 5-6 membered heterocycloalkyl, 5-6 membered heterocycloalkyl(C₁-C₄)alkyl-, or 5-6 membered heteroaryl(C₁-C₄)alkyl-;

provided the compound is not 1-[2-(methyloxy)ethyl]-5′-phenyl-1H, 1H-2,4′-biimidazole;

or a salt, particularly a pharmaceutically acceptable salt, thereof.

The present invention is also directed to a method of inhibiting RIP2 kinase which comprises contacting the kinase with a compound or salt, thereof, according to Formula (I-A).

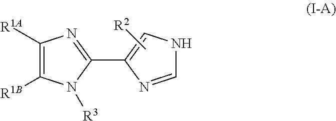

wherein:

R^{1A} and R^{1B} are each independently selected from H and a (C₁-C₆)alkyl group,

or R^{1A} and R^{1B}, taken together with the atoms through which they are attached, form a 6-membered non-aromatic carbocyclic ring or an optionally substituted 6-membered aromatic carbocyclic or heterocyclic ring,

wherein the 6-membered aromatic heterocyclic ring contains one or two nitrogen heteroatoms, and the 6-membered aromatic carbocyclic or heterocyclic ring is optionally substituted by 1-3 substituents each independently selected from halogen, cyano, (C₁-C₄)alkyl, halo(C₁-C₆)alkyl, hydroxy(C₁-C₆)alkyl, (C₁-C₄)alkoxy, halo(C₁-C₆)alkoxy, (C₁-C₄ alkyl)amino-, hydroxy(C₂-C₄ alkyl)amino-, (C₁-C₆ alkyl)(C₁-C₄ alkyl)amino-, (hydroxy(C₂-C₄ alkyl))(C₁-C₄ alkyl)amino-, —CO₂H, —CO₂(C₁-C₄alkyl), —CONH₂, —CONH(C₁-C₄alkyl), —CON(C₁-C₄alkyl)(C₁-C₆alkyl), —CONH(aryl), —CONH(heteroaryl), —SO₂NH₂, —SO₂NH(C₁-C₄alkyl), —SO₂NH(—C₁-C₄alkyl-phenyl), —SO₂N(C₁-C₄alkyl)(C₁-C₆alkyl), heterocycloalkyl, —CO(heterocycloalkyl), and —SO₂(heterocycloalkyl),

wherein any of said heterocycloalkyl is optionally substituted by 1-3 substituents each independently selected from hydroxy, (C₁-C₆)alkyl and hydroxy(C₁-C₄)alkyl,

any of said (C₁-C₄alkyl) is optionally substituted by 1-3 substituents each independently selected from (C₁-C₆)alkoxy, heterocycloalkyl, amino, (C₁-C₄ alkyl)amino-, and (C₁-C₄ alkyl)(C₁-C₄ alkyl)amino-, and

said aryl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy;

R² is monocyclic or bicyclic aryl or monocyclic or bicyclic heteroaryl, optionally substituted by one, two or three R^{2A} substituents,

wherein each R^{2A} is independently selected from halogen, cyano, (C₁-C₄)alkyl, halo(C₁-C₄)alkyl, C₁-C₄ alkoxy, hydroxyl, —CO₂H, —CO₂(C₁-C₄)alkyl, —CONH₂, —CONH(C₁-C₄alkyl), —CON(C₁-C₄alkyl)(C₁-C₄alkyl), phenylC₁-C₄ alkoxy, C₁-C₄ alkylthio-, SO₂(C₁-C₄)alkyl, —SO₂NH₂, —SO₂NH(C₁-C₄alkyl), —SO₂N(C₁-C₄alkyl)(C₁-C₄alkyl), and monocyclic or bicyclic heteroaryl optionally substituted by (C₁-C₄)alkyl;

R³ is (C₁-C₄)alkyl, (C₁-C₂)alkoxy(C₁-C₄)alkyl-, hydroxy(C₂-C₄)alkyl-, 5-6 membered heterocycloalkyl, 5-6 membered heterocycloalkyl(C₁-C₄)alkyl-, or 5-6 membered heteroaryl(C₁-C₄)alkyl-;

or a salt, particularly a pharmaceutically acceptable salt, thereof.

The compounds of the invention are inhibitors of RIP2 kinase and can be useful for the treatment of RIP2-mediated diseases and disorders, particularly uveitis, dermatitis, arthritis, Crohn's disease, asthma, early-onset and extra-intestinal inflammatory bowel disease, and granulomateous disorders, such as adult sarcoidosis, Blau syndrome, early-onset sarcoidosis, and Wegner's Granulomatosis. Accordingly, the invention is also directed to methods of inhibiting RIP2 kinase and treatment of conditions associated therewith using a compound of the invention or a pharmaceutical composition comprising a compound of the invention. The invention is further directed to pharmaceutical compositions comprising a compound of the invention.

### DETAILED DESCRIPTION OF THE INVENTION

The alternative definitions for the various groups and substituent groups of Formula I provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

It will be appreciated by those skilled in the art that the compounds of this invention, represented by generic Formula (I) may exist as tautomers. For example, one preferred embodiment of the compounds of this invention may be represented by the formula:

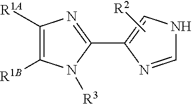

which may exist as imidazole tautomers, represented by Formula (I) and Formula (II):

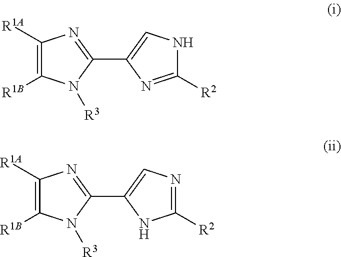

In addition, it will be appreciated by those skilled in the art that the compounds of this invention, depending on further substitution, may exist in other tautomeric forms. All tautomeric forms of the compounds described herein are intended to be encompassed within the scope of the present invention. It is to be understood that any reference to a named compound of this invention is intended to encompass all tautomers of the named compound and any mixtures of tautomers of the named compound.

In one embodiment of this invention, R^{1A} and R^{1B} are each independently selected from H, methyl and ethyl.

In another embodiment, R^{1A} and R^{1B}, taken together with the atoms through which they are attached form a 6-membered non-aromatic carbocyclic group; specifically, R^{1A} and R^{1B} are —CH₂CH₂CH₂CH₂—.

In another embodiment, R^{1A} and R^{1B}, taken together with the atoms through which they are attached, form a 6-membered aromatic carbocyclic or heterocyclic ring, wherein the 6-membered aromatic carbocyclic or heterocyclic ring is optionally substituted by 1-3 substituents each independently selected from halogen, (C₁-C₄)alkyl, halo(C₁-C₆)alkyl, (C₁-C₄)alkoxy, halo(C₁-C₆)alkoxy, —CO₂H, —CO₂(C₁-C₄alkyl), —CONH₂, —CONH(C₁-C₄alkyl), —CON(C₁-C₄alkyl)(C₁-C₆alkyl), —CONH(aryl), —CONH(heteroaryl), —SO₂NH₂, —SO₂NH(C₁-C₄alkyl), and —SO₂N(C₁-C₄alkyl)(C₁-C₆alkyl),

wherein any of said (C₁-C₄alkyl) is optionally substituted by 1-3 substituents each independently selected from (C₁-C₆)alkoxy, heterocycloalkyl, amino, (C₁-C₄ alkyl)amino-, and (C₁-C₄ alkyl)(C₁-C₄ alkyl)amino-,

and said aryl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy.

In a more specific embodiment of the groups defined above, said heteroaryl is a 5-6 membered aromatic ring containing one heteroatom selected from N, O and S, or containing one nitrogen atom and one additional heteroatom selected from N, O and S, said aryl is phenyl, and any of said heterocycloalkyl is a 4-7 membered non-aromatic ring containing one heteroatom selected from N, O and S, or containing one nitrogen atom and one additional heteroatom selected from N, O and S.

In yet another embodiment, when R^{1A} and R^{1B}, taken together with the atoms through which they are attached form a 6-membered aromatic carbocyclic or heterocyclic ring, the 6-membered aromatic carbocyclic or heterocyclic ring is optionally substituted by one, two or three groups each independently selected from halogen, cyano, (C₁-C₄)alkyl, halo(C₁-C₆)alkyl, hydroxy(C₁-C₄)alkyl, (C₁-C₄)alkoxy, halo(C₁-C₄)alkoxy, (C₁-C₄ alkyl)amino-, hydroxy(C₂-C₄ alkyl)amino-, (C₁-C₄)alkoxy-(C₂-C₄ alkyl)amino-, (C₁-C₄ alkyl)(C₁-C₄ alkyl)amino-, (hydroxy(C₂-C₄ alkyl))(C₁-C₄ alkyl)amino-, ((C₁-C₄)alkoxy-(C₂-C₄ alkyl))(C₁-C₄ alkyl)amino-, —CO₂H, —CO₂(C₁-C₄alkyl), heterocycloalkyl, —CO(heterocycloalkyl), —SO₂(heterocycloalkyl), —CONH₂, (C₁-C₄ alkyl)HNCO—, ((C₁-C₄)alkoxy-(C₂-C₄)alkyl)HNCO—, (heterocycloalkyl-(C₁-C₄)alkyl)HNCO—, (C₁-C₄ alkyl)(C₁-C₄ alkyl)NCO—, ((C₁-C₄)alkoxy-(C₂-C₄)alkyl)(C₁-C₄ alkyl)NCO—, —CONH(phenyl), —CONH(heteroaryl), —SO₂NH₂, (C₁-C₄ alkyl)HNSO₂—, ((C₁-C₄)alkoxy-(C₂-C₄)alkyl)HNSO₂—, (phenyl(C₁-C₄)alkyl)HNSO₂—, (C₁-C₄alkyl)(C₁-C₄ alkyl)NSO₂—, and ((C₁-C₄)alkoxy-(C₂-C₄)alkyl)(C₁-C₄alkyl)NSO₂—,

said heteroaryl is a 5-6 membered aromatic ring containing one heteroatom selected from N, O and S, or containing one nitrogen atom and one additional heteroatom selected from N, O and S, said phenyl or heteroaryl is optionally substituted by 1-3 substituents independently selected from halogen, (C₁-C₄)alkyl, halo(C₁-C₄)alkyl, (C₁-C₄)alkoxy, and halo(C₁-C₄)alkoxy, and

any of said heterocycloalkyl is a 4-7 membered non-aromatic ring containing one heteroatom selected from N, O and S, or containing one nitrogen atom and one additional heteroatom selected from N, O and S; which heterocycloalkyl is optionally substituted by 1-2 substituents each independently selected from hydroxy, (C₁-C₆)alkyl and hydroxy(C₁-C₄)alkyl.

In another embodiment, R^{1A} and R^{1B}, taken together with the atoms through which they are attached form a 6-membered aromatic carbocyclic or heterocyclic ring. In this embodiment, the invention is directed to compounds according to Formula (I-B):

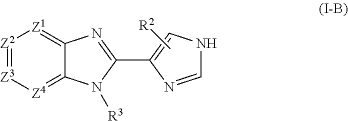

wherein:

each Z¹, Z², Z³, and Z⁴ is independently selected from CH and CR¹; or any one or two of Z¹, Z², Z³, and Z⁴ is N, and each of the remaining two or three of Z¹, Z², Z³, and Z⁴ are independently selected from CH and CR¹;

each R¹ is independently selected from halogen, cyano, (C₁-C₄)alkyl, halo(C₁-C₆)alkyl, hydroxy(C₁-C₆)alkyl, (C₁-C₄)alkoxy, halo(C₁-C₆)alkoxy, (C₁-C₄ alkyl)amino-, hydroxy(C₂-C₄ alkyl)amino-, (C₁-C₆ alkyl)(C₁-C₄ alkyl)amino-, (hydroxy(C₂-C₄ alkyl))(C₁-C₄ alkyl)amino-, —CO₂H, —CO₂(C₁-C₄alkyl), —CONH₂, —CONH(C₁-C₄alkyl), —CON(C₁-C₄alkyl)(C₁-C₆alkyl), —CONH(aryl), —CONH(heteroaryl), —SO₂NH₂, —SO₂NH(C₁-C₄alkyl), —SO₂NH(—C₁-C₄alkyl-phenyl), —SO₂N(C₁-C₄alkyl)(C₁-C₆alkyl), heterocycloalkyl, —CO(heterocycloalkyl), and —SO₂(heterocycloalkyl),

wherein any of said heterocycloalkyl is optionally substituted by 1-3 substituents each independently selected from hydroxy, (C₁-C₆)alkyl and hydroxy(C₁-C₄)alkyl,

any of said (C₁-C₄alkyl) is optionally substituted by 1-3 substituents independently selected from (C₁-C₆)alkoxy, heterocycloalkyl, amino, (C₁-C₄ alkyl)amino-, and (C₁-C₄ alkyl)(C₁-C₄ alkyl)amino-, and

said aryl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy;

and R² and R³ are as defined herein.

In another embodiment of the compound of Formula (I-B), each R¹ is independently selected from halogen, (C₁-C₄)alkyl, halo(C₁-C₆)alkyl, (C₁-C₄)alkoxy, halo(C₁-C₆)alkoxy, —CO₂H, —CO₂(C₁-C₄alkyl), —CONH₂, —CONH(C₁-C₄alkyl), —CON(C₁-C₄alkyl)(C₁-C₆alkyl), —CONH(aryl), —CONH(heteroaryl), —SO₂NH₂, —SO₂NH(C₁-C₄alkyl), and —SO₂N(C₁-C₄alkyl)(C₁-C₆alkyl),

wherein any of said (C₁-C₄alkyl) is optionally substituted by 1-3 substituents each independently selected from (C₁-C₆)alkoxy, heterocycloalkyl, amino, (C₁-C₄ alkyl)amino-, and (C₁-C₄ alkyl)(C₁-C₄ alkyl)amino-,

and said aryl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy;

or a salt, particularly a pharmaceutically acceptable salt, thereof.

In another embodiment of the compound of Formula (I-B), said heteroaryl is a 5-6 membered aromatic ring containing one heteroatom selected from N, O and S, or containing one nitrogen atom and one additional heteroatom selected from N, O and S, said aryl is phenyl, and any of said heterocycloalkyl is a 4-7 membered non-aromatic ring containing one heteroatom selected from N, O and S, or containing one nitrogen atom and one additional heteroatom selected from N, O and S.

In another embodiment of the compound of Formula (I-B), each R¹ is independently selected from halogen, cyano, (C₁-C₄)alkyl, halo(C₁-C₆)alkyl, hydroxy(C₁-C₄)alkyl, (C₁-C₄)alkoxy, halo(C₁-C₄)alkoxy, (C₁-C₄ alkyl)amino-, hydroxy(C₂-C₄ alkyl)amino-, (C₁-C₄)alkoxy-(C₂-C₄ alkyl)amino-, (C₁-C₄ alkyl)(C₁-C₄ alkyl)amino-, (hydroxy(C₂-C₄ alkyl))(C₁-C₄ alkyl)amino-, ((C₁-C₄)alkoxy-(C₂-C₄ alkyl))(C₁-C₄ alkyl)amino-, —CO₂H, —CO₂(C₁-C₄alkyl), heterocycloalkyl, —CO(heterocycloalkyl), —SO₂(heterocycloalkyl), —CONH₂, (C₁-C₄ alkyl)HNCO—, ((C₁-C₄)alkoxy-(C₂-C₄)alkyl)HNCO—, (heterocycloalkyl-(C₁-C₄)alkyl)HNCO—, (C₁-C₄ alkyl)(C₁-C₄ alkyl)NCO—, ((C₁-C₄)alkoxy-(C₂-C₄)alkyl)(C₁-C₄ alkyl)NCO—, —CONH(phenyl), —CONH(heteroaryl), —SO₂NH₂, (C₁-C₄ alkyl)HNSO₂—, ((C₁-C₄)alkoxy-(C₂-C₄)alkyl)HNSO₂—, (phenyl(C₁-C₄)alkyl)HNSO₂—, (C₁-C₄alkyl)(C₁-C₄ alkyl)NSO₂—, and ((C₁-C₄)alkoxy-(C₂-C₄)alkyl)(C₁-C₄alkyl)NSO₂—,

said heteroaryl is a 5-6 membered aromatic ring containing one heteroatom selected from N, O and S, or containing one nitrogen atom and one additional heteroatom selected from N, O and S, said phenyl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy, and

any of said heterocycloalkyl is a 4-7 membered non-aromatic ring containing one heteroatom selected from N, O and S, or containing one nitrogen atom and one additional heteroatom selected from N, O and S; which heterocycloalkyl is optionally substituted by 1-2 substituents each independently selected from hydroxy, (C₁-C₆)alkyl and hydroxy(C₁-C₄)alkyl.

In one embodiment of the compound of Formula (I-B), each of Z¹, Z², Z³, and Z⁴ is CH.

In another embodiment, one of Z² or Z³ is CR¹ and Z¹, Z⁴ and the other of Z² or Z³ is CH.

In yet another embodiment, Z² and Z³ are CR¹ and Z¹ and Z⁴ are CH.

In a further embodiment, any one of Z¹, Z², Z³, and Z⁴ is N, and each of the remaining three of Z¹, Z², Z³, L and Z⁴ is CH. Specifically Z² is N, and Z¹, Z³, and Z⁴ are CH or Z⁴ is N, and Z¹, Z², and Z³ are CH.

In a still further embodiment, any one of Z¹, Z², Z³, and Z⁴ is N, one of the remaining Z¹, Z², Z³, and Z⁴ is CR¹; and the remaining two of Z¹, Z², Z³, and Z⁴ is CH. Specifically Z² is N, Z¹ is CR¹ and Z³, and Z⁴ are CH; or Z² is N, Z′ and Z³ are CR¹ and Z⁴ is CH; or Z⁴ is N, Z³ is CR¹ and Z¹ and Z² are CH.

In other specific embodiments, each R¹ is independently selected from halogen, cyano, (C₁-C₄)alkyl, halo(C₁-C₆)alkyl, hydroxy(C₁-C₄)alkyl, (C₁-C₄)alkoxy, (hydroxy(C₂-C₄ alkyl))(C₁-C₄ alkyl)amino-, ((C₁-C₄)alkoxy-(C₂-C₄ alkyl))(C₁-C₄ alkyl)amino-, —CO₂H, —CO₂(C₁-C₄alkyl), heterocycloalkyl, —CO(heterocycloalkyl), —SO₂(heterocycloalkyl), (C₁-C₄ alkyl)HNCO—, ((C₁-C₄)alkoxy-(C₂-C₄)alkyl)HNCO—, (heterocycloalkyl-(C₁-C₄)alkyl)HNCO—, (C₁-C₄ alkyl)(C₁-C₄ alkyl)NCO—, —CONH(heteroaryl), —SO₂NH₂, (C₁-C₄ alkyl)HNSO₂—, ((C₁-C₄)alkoxy-(C₂-C₄)alkyl)HNSO₂—, and (Phenyl(C₁-C₄)alkyl)HNSO₂,

wherein said heteroaryl is a 6-membered aromatic heterocyclic ring containing one or two nitrogen heteroatoms which ring is optionally substituted by 1-2 substituents each independently selected from halogen, (C₁-C₄)alkyl and (C₁-C₄)alkoxy, and

said heterocycloalkyl is a 4-6 membered non-aromatic heterocyclic ring containing one heteroatom selected from N, O and S, or containing one nitrogen atoms and one additional heteroatom selected from N, O and S, which ring is optionally substituted by 1-2 substituents each independently selected from hydroxy, (C₁-C₆)alkyl and hydroxy(C₁-C₄)alkyl.

In other specific embodiments, R¹ is methyl, chloro, fluoro, bromo, cyano, trifluoromethyl, methoxy, ethoxy, —CH₂OH, —C(CH₃)₂OH, —CO₂H, —CO₂CH₃, —SO₂CH₃, —SO₂-benzyl, —SO₂NH₂, —CONHCH₃, —CON(CH₃)₂, —CONHCH₂CH₂OCH₃, —CONHCH₂CH₂(morpholin-4-yl), —CONH-pyrid-2-yl, —CONH-pyrid-3-yl, —CONH-pyrid-4-yl, morpholin-4-yl-CO—, [(3R)-3-methyl-morpholin-4-yl]-CO—, [(3S)-3-methyl-morpholin-4-yl]-CO—, (4-methyl-piperazin-1-yl)-CO—, morpholin-4-yl, (3S)-3-methyl-morpholin-4-yl, (3R)-3-methyl-morpholin-4-yl, (3R)-3-ethyl-morpholin-4-yl, 2-methyl-morpholin-4-yl, ((2S,5R)-5-ethyl-2-hydroxymethyl-morpholin-4-yl, ((2S,5S)-5-methyl-2-hydroxymethyl-morpholin-4-yl, piperidin-1-yl, pyrrolidin-1-yl, azetidin-1-yl, 4-methyl-piperazin-1-yl, (3S)-3,4-dimethyl-piperazin-1-yl, (2-(methoxy)ethyl)(methyl)amino-, (2-(methoxy)ethyl)(ethyl)amino-, (2-(hydroxyl)ethyl)(methyl)amino-, morpholin-4-yl-SO₂—, pyrrolidin-1-yl-SO₂—, piperazin-1-yl-SO₂—, 4-methyl-piperazin-1-yl-SO₂—, and (2-(methoxy)ethyl)HNSO₂—.

In another embodiment of this invention, each R¹ is independently selected from halo, (C₁-C₄)alkyl, halo(C₁-C₆)alkyl, (C₁-C₄)alkoxy, (C₁-C₄)alkoxy(C₁-C₄)alkyl-, (C₁-C₄)alkoxy(C₂-C₄)alkoxy-, —CO₂H, —CO₂(C₁-C₄alkyl), —CONH(C₁-C₄alkyl), CON(C₁-C₄alkyl)(C₁-C₆alkyl), and —CONH(heteroaryl), where any of said (C₁-C₄alkyl) is optionally substituted by (C₁-C₆)alkoxy or heterocycloalkyl, and said heteroaryl is optionally substituted by 1-2 substituents each independently selected from halogen, (C₁-C₄)alkyl and (C₁-C₄)alkoxy.

In other specific embodiments, R¹ is methyl, chloro, trifluoromethyl, methoxy, —CO₂H, —CO₂CH₃, —CONHCH₃, —CON(CH₃)₂, —CONHCH₂CH₂OCH₃, —CONHCH₂CH₂(morpholin-4-yl), —CONH-pyrid-2-yl, —CONH-pyrid-3-yl, or —CONH-pyrid-4-yl.

In yet another embodiment of this invention, one of Z² or Z³ is CR¹ and R¹ is halo, (C₁-C₄)alkyl, halo(C₁-C₄)alkyl, (C₁-C₄)alkoxy, —CO₂H, —CO₂(C₁-C₄)alkyl, ((C₁-C₄)alkyl)NHCO—, ((C₁-C₄)alkyl)((C₁-C₄)alkyl)NCO—, ((C₁-C₄)alkoxy(C₂-C₄)alkyl)NHCO—, ((C₁-C₄)alkoxy(C₂-C₄)alkyl)((C₁-C₄)alkyl)NCO—, (5-6 membered heterocycloalkyl-(C₁-C₄)alkyl)NHCO—, (5-6 membered heterocycloalkyl-(C₁-C₄)alkyl)((C₁-C₄)alkyl)NCO—, (heteroaryl)NHCO—, or (heteroaryl(C₁-C₄)alkyl)NHCO—.

Accordingly, in an embodiment of the compounds of Formula (I) or (I-A), when R^{1A} and R^{1B}, taken together with the atoms through which they are attached form a 6-membered aromatic carbocyclic or heterocyclic ring, the carbocyclic or heterocyclic ring is optionally substituted by one or two groups each independently selected from halogen, cyano, (C₁-C₄)alkyl, halo(C₁-C₆)alkyl, hydroxy(C₁-C₄)alkyl, (C₁-C₄)alkoxy, (hydroxy(C₂-C₄ alkyl))(C₁-C₄ alkyl)amino-, ((C₁-C₄)alkoxy-(C₂-C₄ alkyl))(C₁-C₄ alkyl)amino-, —CO₂H, —CO₂(C₁-C₄alkyl), heterocycloalkyl, —CO(heterocycloalkyl), —SO₂(heterocycloalkyl), (C₁-C₄ alkyl)HNCO—, ((C₁-C₄)alkoxy-(C₂-C₄)alkyl)HNCO—, (heterocycloalkyl-(C₁-C₄)alkyl)HNCO—, (C₁-C₄ alkyl)(C₁-C₄ alkyl)NCO—, —CONH(heteroaryl), —SO₂NH₂, (C₁-C₄ alkyl)HNSO₂—, ((C₁-C₄)alkoxy-(C₂-C₄)alkyl)HNSO₂—, and (phenyl(C₁-C₄)alkyl)HNSO₂,

wherein said heteroaryl is a 6-membered aromatic heterocyclic ring containing one or two nitrogen heteroatoms which ring is optionally substituted by 1-2 substituents each independently selected from halogen, (C₁-C₄)alkyl and (C₁-C₄)alkoxy, and

said heterocycloalkyl is a 5-6 membered non-aromatic heterocyclic ring containing one heteroatom selected from N, O and S, or containing one nitrogen atoms and one additional heteroatom selected from N, O and S, which ring is optionally substituted by 1-2 substituents each independently selected from hydroxy, (C₁-C₆)alkyl and hydroxy(C₁-C₄)alkyl.

For the compounds of Formula (I) or (I-A), when R^{1A} and R^{1B}, taken together with the atoms through which they are attached form a 6-membered aromatic carbocyclic or heterocyclic ring, the carbocyclic or heterocyclic ring is optionally substituted by one or two groups each independently selected from halogen, methyl, chloro, fluoro, bromo, cyano, trifluoromethyl, methoxy, ethoxy, —CH₂OH, —C(CH₃)₂OH, —CO₂H, —CO₂CH₃, —SO₂CH₃, —SO₂-benzyl, —SO₂NH₂, —CONHCH₃, —CON(CH₃)₂, —CONHCH₂CH₂OCH₃, —CONHCH₂CH₂(morpholin-4-yl), —CONH-pyrid-2-yl, —CONH-pyrid-3-yl, —CONH-pyrid-4-yl, morpholin-4-yl-CO—, [(3R)-3-methyl-morpholin-4-yl]-CO—, [(3S)-3-methyl-morpholin-4-yl]-CO—, (4-methyl-piperazin-1-yl)-CO—, morpholin-4-yl, (3S)-3-methyl-morpholin-4-yl, (3R)-3-methyl-morpholin-4-yl, (3R)-3-ethyl-morpholin-4-yl, 2-methyl-morpholin-4-yl, ((2S,5R)-5-ethyl-2-hydroxymethyl-morpholin-4-yl, ((2S,5S)-5-methyl-2-hydroxymethyl-morpholin-4-yl, piperidin-1-yl, pyrrolidin-1-yl, azetidin-1-yl, 4-methyl-piperazin-1-yl, (3S)-3,4-dimethyl-piperazin-1-yl, (2-(methoxy)ethyl)(methyl)amino-, (2-(methoxy)ethyl)(ethyl)amino-, (2-(hydroxyl)ethyl)(methyl)amino-, morpholin-4-yl-SO₂—, pyrrolidin-1-yl-SO₂—, piperazin-1-yl-SO₂—, 4-methyl-piperazin-1-yl-SO₂—, and (2-(methoxy)ethyl)HNSO₂—.

In specific embodiments of this invention one of Z² or Z³ is CR¹ and R¹ is methyl, chloro, trifluoromethyl, methoxy, —CO₂H, —CO₂CH₃, —CONHCH₃, —CON(CH₃)₂, —CONHCH₂CH₂OCH₃, —CONHCH₂CH₂(morpholin-4-yl), —CONH-pyrid-2-yl, —CONH-pyrid-3-yl, or —CONH-pyrid-4-yl.

In one embodiment of this invention, R² is a 5-6 membered, monocyclic or a 9-10 membered, bicyclic heteroaryl, wherein said heteroaryl contains one heteroatom selected from N, O and S, or contains one or two nitrogen atoms and one additional heteroatom selected from N, O and S.

In another embodiment, R² is a 6-membered heteroaryl containing one or two nitrogen heteroatoms. In another embodiment, R² is a 9-membered heteroaryl containing one or two nitrogen heteroatoms and optionally containing one additional heteroatom selected from O and S. Specifically, R² is a 9-membered bicyclic heteroaryl, wherein the 5-membered ring moiety thereof contains one or two nitrogen heteroatoms or contains one nitrogen atom and one sulfur atom or contains two nitrogen atoms and one oxygen atom.

In another embodiment of this invention, R² is phenyl or a 5-6 membered or a 9-10 membered heteroaryl, optionally substituted one, two or three times with R^{2A}, where each R^{2A} is independently selected from halogen, (C₁-C₄)alkyl, halo(C₁-C₄)alkyl, hydroxyl, C₁-C₄ alkoxy, —CONH₂, —CONH(C₁-C₄alkyl), —CON(C₁-C₄alkyl)(C₁-C₄alkyl), —SO₂(C₁-C₄)alkyl, —SO₂NH₂, —SO₂NH(C₁-C₄alkyl), and a 5-6 membered heteroaryl optionally substituted by (C₁-C₄)alkyl.

In selected embodiments, R² is phenyl, pyridyl, benzothiazolyl, indolyl, indazolyl, or benzoxadiazolyl, each optionally substituted by one R^{2A} and further optionally substituted by a second R^{2A}.

In other selected embodiments, each R^{2A} is independently selected from chloro, fluoro, methyl, trifluoromethyl, hydroxyl, methoxy, —CON(CH₃)₂, —CONH₂, —SO₂NH₂, and —SO₂CH₃.

More specifically, R² is phenyl, pyridyl, benzothiazolyl, indolyl, indazolyl, or benzoxadiazolyl, each optionally substituted by one R^{2A} selected from chloro, fluoro, methyl, trifluoromethyl, hydroxyl, methoxy, —CON(CH₃)₂, —CONH₂, —SO₂NH₂, and —SO₂CH₃, and further optionally substituted by a second R^{2A} selected from chloro, fluoro, and methoxy.

In more specific embodiments, R² is an unsubstituted benzothiazolyl (specifically, 1,3-benzothiazol-5-yl), indolyl (specifically, indol-5-yl and indol-6-yl), indazolyl (specifically, indazol-5-yl and indazol-6-yl) or benzoxadiazolyl (specifically, 2,1,3-benzoxadiazol-5-yl) group, or R² is an optionally substituted phenyl group, optionally substituted by one R^{2A} selected from chloro, fluoro, methoxy, —CON(CH₃)₂, —CONH₂, —SO₂NH₂, and —SO₂CH₃, and further optionally substituted by a second R^{2A} selected from chloro, fluoro, and methoxy.

In other embodiments, R² is 1,3-benzothiazol-5-yl, indol-5-yl, indol-6-yl, indazol-5-yl indazol-6-yl, 2,1,3-benzoxadiazol-5-yl, phenyl, 3-chlorophenyl, 4-chlorophenyl, 2-methoxyphenyl, 3-methoxyphenyl, 4-methoxyphenyl, 2,5-dimethoxyphenyl, 2-fluoro-5-methoxyphenyl, 4-chloro-3-methoxyphenyl, 4-chloro-3-hydroxyphenyl, 3-(H₂NCO)phenyl, 3-(H₂NSO₂)phenyl, 4-(H₂NSO₂)phenyl, or 4-(CH₃SO₂)phenyl; more specifically, R² is 1,3-benzothiazol-5-yl or 4-chlorophenyl.

In another embodiment of this invention, R³ is (C₁-C₄)alkyl, (C₁-C₂)alkoxy(C₁-C₃)alkyl-, hydroxy(C₂-C₃)alkyl-, 5 membered heterocycloalkyl, 5-6 membered heterocycloalkyl(C₁-C₃)alkyl-, or 5-6 membered heteroaryl(C₁-C₃)alkyl-. In other embodiments, R³ is (C₁)alkoxy(C₁-C₃)alkyl-.

In specific embodiments, R³ is —CH₂CH₂CH₃, —CH₂CH₂OCH₃, —CH₂CH₂OCH₂CH₃, —CH₂CH₂OH, tetrahydrofuran-3-yl, —CH₂-tetrahydrofuran-2-yl, —CH₂-pyrid-2-yl, or —CH₂CH₂-pyrid-2-yl, more specifically, R³ is —CH₂CH₂OCH₃.

The invention is more specifically directed to a compound according to Formula (I), wherein: Z² is CR¹, and Z¹, Z³, and Z⁴ are CH; R¹ is halo, (C₁-C₄)alkyl, halo(C₁-C₄)alkyl, (C₁-C₄)alkoxy, —CO₂H, —CO₂(C₁-C₄)alkyl, ((C₁-C₄)alkyl)NHCO—, ((C₁-C₄)alkyl)((C₁-C₄)alkyl)NCO—, ((C₁-C₄)alkoxy(C₂-C₄)alkyl)NHCO—, ((C₁-C₄)alkoxy(C₂-C₄)alkyl)((C₁-C₄)alkyl)NCO—, (5-6 membered heterocycloalkyl-(C₁-C₄)alkyl)NHCO—, (5-6 membered heterocycloalkyl-(C₁-C₄)alkyl)((C₁-C₄)alkyl)NCO—, (heteroaryl)NHCO—, or (heteroaryl(C₁-C₄)alkyl)NHCO—; R² is phenyl or a 5-6 membered or a 9-10 membered heteroaryl, optionally substituted one, two or three times with R^{2A}, where each R^{2A} is independently selected from halogen, (C₁-C₄)alkyl, halo(C₁-C₄)alkyl, hydroxyl, C₁-C₄ alkoxy, —CONH₂, —CONH(C₁-C₄alkyl), —CON(C₁-C₄alkyl)(C₁-C₄alkyl), —SO₂(C₁-C₄)alkyl, —SO₂NH₂, —SO₂NH(C₁-C₄alkyl), and a 5-6 membered heteroaryl optionally substituted by (C₁-C₄)alkyl; R³ is (C₁-C₄)alkyl, (C₁)alkoxy(C₁-C₃)alkyl-, hydroxy(C₂-C₃)alkyl-, 5 membered heterocycloalkyl, 5-6 membered heterocycloalkyl(C₁-C₃)alkyl-, or 5-6 membered heteroaryl(C₁-C₃)alkyl-; or a salt, particularly a pharmaceutically acceptable salt, thereof.

In another embodiment of the compounds of Formula (I), Z² is CR¹, and Z¹, Z³, and Z⁴ are CH, where R¹ is methyl, chloro, trifluoromethyl, methoxy, —CO₂H, —CO₂CH₃, —CONHCH₃, —CON(CH₃)₂, —CONHCH₂CH₂OCH₃, —CONHCH₂CH₂(morpholin-4-yl), —CONH-pyrid-2-yl, —CONH-pyrid-3-yl, or —CONH-pyrid-4-yl; R² is unsubstituted 1,3-benzothiazol-5-yl, indol-5-yl, indol-6-yl, indazol-5-yl, indazol-6-yl, or benzoxadiazolyl, or phenyl, substituted by chloro, fluoro, methoxy, —CON(CH₃)₂, —CONH₂, —SO₂NH₂, or —SO₂CH₃, and further optionally substituted by a second group selected from chloro, fluoro, and methoxy; and R³ is —CH₂CH₂CH₃, —CH₂CH₂OCH₃, —CH₂CH₂OCH₂CH₃, —CH₂CH₂OH, tetrahydrofuran-3-yl, —CH₂-tetrahydrofuran-2-yl, —CH₂-pyrid-2-yl, or —CH₂CH₂-pyrid-2-yl; or a salt, particularly a pharmaceutically acceptable salt, thereof.

This invention is further directed to a compound according to Formula (I-C):

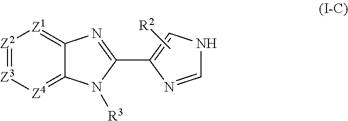

wherein Z¹, Z², Z³, Z⁴, R¹, R² and R³ are as defined herein, in any combination of said definitions, or a salt, particularly a pharmaceutically acceptable salt, thereof.

The invention is further directed to a compound according to Formula (II):

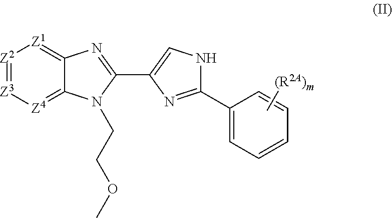

wherein m is 1, 2 or 3, Z¹, Z², Z³, Z⁴, R¹ and R^{2A} are as defined herein, in any combination of said definitions,

or a salt, particularly a pharmaceutically acceptable salt, thereof.

In another embodiment, the invention is directed to a compound according to Formula (III):

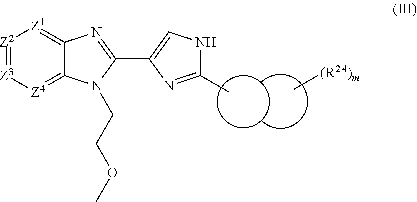

wherein m is 1, 2 or 3,

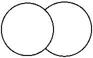

is a 9-10 membered heteroaryl, and

Z¹, Z², Z³, Z⁴, R¹ and R^{2A} are as defined herein, in any combination of said definitions,

or a salt, particularly a pharmaceutically acceptable salt, thereof.

In a further embodiment, the invention is directed to a compound or salt according to Formula (II) or Formula (III), wherein:

one of Z² or Z³ is CR¹ and Z¹, Z⁴ and the other of Z² or Z³ is CH, or

Z² and Z³ are CR¹ and Z′ and Z⁴ are CH, or

any one of Z¹, Z², Z³, and Z⁴ is N, and each of the remaining three of Z¹, Z², Z³, and Z⁴ is CH, or

any one of Z¹, Z², Z³, and Z⁴ is N, one of the remaining Z¹, Z², Z³, and Z⁴ is CR¹; and the remaining two of Z¹, Z², Z³, and Z⁴ is CH;

each R¹ is independently selected from halogen, (C₁-C₄)alkyl, halo(C₁-C₆)alkyl, (C₁-C₄)alkoxy, halo(C₁-C₆)alkoxy, —CO₂H, —CO₂(C₁-C₄alkyl), —CONH₂, —CONH(C₁-C₄alkyl), CON(C₁-C₄alkyl)(C₁-C₈alkyl), —CONH(aryl), —CONH(heteroaryl), —SO₂NH₂, —SO₂NH(C₁-C₄alkyl), and —SO₂N(C₁-C₄alkyl)(C₁-C₄)alkyl),

wherein any of said (C₁-C₄alkyl) is optionally substituted by 1-3 substituents each independently selected from (C₁-C₆)alkoxy, heterocycloalkyl, amino, (C₁-C₄ alkyl)amino-, and (C₁-C₄ alkyl)(C₁-C₄ alkyl)amino-,

and said aryl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy;

m is 0, 1, 2 or 3 and each R^{2A} is independently selected from halogen, cyano, (C₁-C₄)alkyl, halo(C₁-C₄)alkyl, C₁-C₄ alkoxy, hydroxyl, —CO₂H, —CO₂(C₁-C₄)alkyl, —CONH₂, —CONH(C₁-C₄alkyl), —CON(C₁-C₄alkyl)(C₁-C₄alkyl), phenylC₁-C₄ alkoxy, C₁-C₄ alkylthio-, —SO₂(C₁-C₄)alkyl, —SO₂NH₂, —SO₂NH(C₁-C₄alkyl), —SO₂N(C₁-C₄alkyl)(C₁-C₄alkyl), and monocyclic or bicyclic heteroaryl optionally substituted by (C₁-C₄)alkyl.

Accordingly, a compound of this invention includes a compound of Formula (I), (I-A), (I-B), (I-C), (II) or (III), or a salt thereof, particularly a pharmaceutically acceptable salt thereof.

As used herein, the term “alkyl” represents a saturated, straight or branched hydrocarbon moiety, which may be unsubstituted or substituted by one, or more of the substituents defined herein. Exemplary alkyls include, but are not limited to methyl (Me), ethyl (Et), propyl, isopropyl, butyl, isobutyl, t-butyl and pentyl. The term “C₁-C₄” refers to an alkyl containing from 1 to 4 carbon atoms.

When the term “alkyl” is used in combination with other substituent groups, such as “haloalkyl” or “hydroxyalkyl” or “arylalkyl”, the term “alkyl” is intended to encompass a divalent straight or branched-chain hydrocarbon radical. For example, “arylalkyl” is intended to mean the radical -alkylaryl, wherein the alkyl moiety thereof is a divalent straight or branched-chain carbon radical and the aryl moiety thereof is as defined herein, and is represented by the bonding arrangement present in a benzyl group (—CH₂-phenyl).

As used herein, the term “alkenyl” refers to a straight or branched hydrocarbon moiety containing at least 1 and up to 3 carbon-carbon double bonds. Examples include ethenyl and propenyl.

As used herein, the term “alkynyl” refers to a straight or branched hydrocarbon moiety containing at least 1 and up to 3 carbon-carbon triple bonds. Examples include ethynyl and propynyl.

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” refers to a group containing an alkyl radical attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Alkylthio-” refers to a group containing an alkyl radical attached through a sulfur linking atom. The term “(C₁-C₄)alkylthio-” refers to a straight- or branched-chain hydrocarbon radical having at least 1 and up to 4 carbon atoms attached through a sulfur linking atom. Exemplary “(C₁-C₄)alkylthio-” groups useful in the present invention include, but are not limited to, methylthio-, ethylthio-, n-propylthio-, isopropylthio-, n-butylthio-, s-butylthio-, and t-butylthio-.

“Cycloalkyloxy” and “cycloalkylthio” refers to a group containing a saturated carbocyclic ring atoms attached through an oxygen or sulfur linking atom, respectively. Examples of “cycloalkyloxy” moieties include, but are not limited to, cyclopropoxy, cyclobutoxy, cyclopentyloxy, cyclohexyloxy, and the like.

“Aryl” represents a group or moiety comprising an aromatic, monovalent monocyclic or bicyclic hydrocarbon radical containing from 6 to 10 carbon ring atoms, which may be unsubstituted or substituted by one or more of the substituents defined herein, and to which may be fused one or more cycloalkyl rings, which may be unsubstituted or substituted by one or more substituents defined herein.

Generally, in the compounds of this invention, aryl is phenyl.

Heterocyclic groups may be heteroaryl or heterocycloalkyl groups.

“Heterocycloalkyl” represents a group or moiety comprising a non-aromatic, monovalent monocyclic or bicyclic radical, which is saturated or partially unsaturated, containing 3 to 10 ring atoms, which includes 1 to 4 heteroatoms selected from nitrogen, oxygen and sulfur, and which may be unsubstituted or substituted by one or more of the substituents defined herein. Illustrative examples of heterocycloalkyls include, but are not limited to, azetidinyl, pyrrolidyl (or pyrrolidinyl), piperidinyl, piperazinyl, morpholinyl, tetrahydro-2H-1,4-thiazinyl, tetrahydrofuryl (or tetrahydrofuranyl), dihydrofuryl, oxazolinyl, thiazolinyl, pyrazolinyl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-membered and/or 6-membered heterocycloalkyl groups, such as pyrrolidyl (or pyrrolidinyl), tetrahydrofuryl (or tetrahydrofuranyl), tetrahydrothienyl, dihydrofuryl, oxazolinyl, thiazolinyl or pyrazolinyl, piperidyl (or piperidinyl), piperazinyl, morpholinyl, tetrahydropyranyl, dihydropyranyl, 1,3-dioxanyl, tetrahydro-2H-1,4-thiazinyl, 1,4-dioxanyl, 1,3-oxathianyl, and 1,3-dithianyl.

“Heteroaryl” represents a group or moiety comprising an aromatic monovalent monocyclic or bicyclic radical, containing 5 to 10 ring atoms, including 1 to 4 heteroatoms selected from nitrogen, oxygen and sulfur, which may be unsubstituted or substituted by one or more of the substituents defined herein. This term also encompasses bicyclic heterocyclic-aryl compounds containing an aryl ring moiety fused to a heterocycloalkyl ring moiety, containing 5 to 10 ring atoms, including 1 to 4 heteroatoms selected from nitrogen, oxygen and sulfur, which may be unsubstituted or substituted by one or more of the substituents defined herein. Illustrative examples of heteroaryls include, but are not limited to, thienyl, pyrrolyl, imidazolyl, pyrazolyl, furyl (or furanyl), isothiazolyl, furazanyl, isoxazolyl, oxazolyl, oxadiazolyl, thiazolyl, pyridyl (or pyridinyl), pyrazinyl, pyrimidinyl, pyridazinyl, triazinyl, tetrazinyl, triazolyl, tetrazolyl, benzo[b]thienyl, isobenzofuryl, 2,3-dihydrobenzofuryl, chromenyl, chromanyl, indolizinyl, isoindolyl, indolyl, indazolyl, purinyl, isoquinolyl, quinolyl, phthalazinyl, naphthridinyl, quinzolinyl, benzothiazolyl, benzimidazolyl, tetrahydroquinolinyl, cinnolinyl, pteridinyl, isothiazolyl.

Generally, the heteroaryl groups present in the compounds of this invention are 5-membered and/or 6-membered monocyclic heteroaryl groups. Selected 5-membered heteroaryl groups contain one nitrogen, oxygen or sulfur ring heteroatom, and optionally contain 1, 2 or 3 additional nitrogen ring atoms. Selected 6-membered heteroaryl groups contain 1, 2, 3 or 4 nitrogen ring heteroatoms. Selected 5- or 6-membered heteroaryl groups include thienyl, pyrrolyl, imidazolyl, pyrazolyl, furyl, isothiazolyl, furazanyl, isoxazolyl, oxazolyl, oxadiazolyl, thiazolyl, triazolyl, and tetrazolyl or pyridyl, pyrazinyl, pyrimidinyl, pyridazinyl, and triazinyl.

It is to be understood that the terms heterocycle, heterocyclic, heteroaryl, heterocycloalkyl, are intended to encompass stable heterocyclic groups where a ring nitrogen heteroatom is optionally oxidized (e.g., heterocyclic groups containing an N-oxide, such as pyridine-N-oxide) or where a ring sulfur heteroatom is optionally oxidized (e.g., heterocyclic groups containing sulfones or sulfoxide moieties, such as tetrahydrothienyl-1-oxide (a tetramethylene sulfoxide) or tetrahydrothienyl-1,1-dioxide (a tetramethylene sulfone)).

“Oxo” represents a double-bonded oxygen moiety; for example, if attached directly to a carbon atom forms a carbonyl moiety (C═O). The terms “halogen” and “halo” represent chloro, fluoro, bromo or iodo substituents. “Hydroxy” or “hydroxyl” is intended to mean the radical —OH.

As used herein, the term “compound(s) of the invention” means a compound of Formula (I), (I-A), (I-B), (I-C), (II) or (III) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means unsubstituted groups or rings (e.g., cycloalkyl, heterocycle, and heteroaryl rings) and groups or rings substituted with one or more specified substituents.

Specific compounds of this invention include:
2-[2-(4-chlorophenyl)-1H-imidazol-4-yl]-1-[2-(methyloxy)ethyl]-N-[2-(4-morpholinyl)ethyl]-1H-benzimidazole-5-carboxamide,
2-[2-(4-chlorophenyl)-1H-imidazol-4-yl]-N-methyl-1-(2-pyridinylmethyl)-1H-benzimidazole-5-carboxamide,
2-[2-(4-chlorophenyl)-1H-imidazol-4-yl]-N-methyl-1-(tetrahydro-2-furanylmethyl)-1H-benzimidazole-5-carboxamide,
2-[2-(4-chlorophenyl)-1H-imidazol-4-yl]-N-methyl-1-(tetrahydro-3-furanyl)-1H-benzimidazole-5-carboxamide,
2-[2-(4-chlorophenyl)-1H-imidazol-4-yl]-N-methyl-1-[2-(2-pyridinyl)ethyl]-1H-benzimidazole-5-carboxamide,
2-{2-[2-fluoro-5-(methyloxy)phenyl]-1H-imidazol-4-yl}-N-methyl-1-[2-(methyloxy)ethyl]-1H-benzimidazole-5-carboxamide,
N-methyl-1-[2-(methyloxy)ethyl]-2-{2-[3-(methyloxy)phenyl]-1H-imidazol-4-yl}-1H-benzimidazole-5-carboxamide,
2-{2-[2,5-bis(methyloxy)phenyl]-1H-imidazol-4-yl}-N-methyl-1-[2-(methyloxy)ethyl]-1H-benzimidazole-5-carboxamide,
N-methyl-1-[2-(methyloxy)ethyl]-2-{2-[2-(methyloxy)phenyl]-1H-imidazol-4-yl}-1H-benzimidazole-5-carboxamide,
N-methyl-1-[2-(methyloxy)ethyl]-2-{2-[4-(methyloxy)phenyl]-1H-imidazol-4-yl}-1H-benzimidazole-5-carboxamide,
2-{2-[4-chloro-3-(methyloxy)phenyl]-1H-imidazol-4-yl}-N,N-dimethyl-1-[2-(methyloxy)ethyl]-1H-benzimidazole-5-carboxamide,
2-[2-(4-chloro-3-hydroxyphenyl)-1H-imidazol-4-yl]-N,N-dimethyl-1-[2-(methyloxy)ethyl]-1H-benzimidazole-5-carboxamide,
2-[2-(1,3-benzothiazol-5-yl)-1H-imidazol-4-yl]-N-methyl-1-[2-(methyloxy)ethyl]-1H-benzimidazole-5-carboxamide,
2-{2-[4-chloro-3-(methyloxy)phenyl]-1H-imidazol-4-yl}-N-methyl-1-[2-(methyloxy)ethyl]-1H-benzimidazole-5-carboxamide,
N-methyl-1-[2-(methyloxy)ethyl]-2-{2-[4-(methylsulfonyl)phenyl]-1H-imidazol-4-yl}-1H-benzimidazole-5-carboxamide,
2-{2-[4-(aminosulfonyl)phenyl]-1H-imidazol-4-yl}-N-methyl-1-[2-(methyloxy)ethyl]-1H-benzimidazole-5-carboxamide,
2-[2-(1H-indol-5-yl)-1H-imidazol-4-yl]-N-methyl-1-[2-(methyloxy)ethyl]-1H-benzimidazole-5-carboxamide,
2-[2-(1H-indol-6-yl)-1H-imidazol-4-yl]-N-methyl-1-[2-(methyloxy)ethyl]-1H-benzimidazole-5-carboxamide,
2-{2-[3-(aminosulfonyl)phenyl]-1H-imidazol-4-yl}-N-methyl-1-[2-(methyloxy)ethyl]-1H-benzimidazole-5-carboxamide,
2-{2-[3-(aminocarbonyl)phenyl]-1H-imidazol-4-yl}-N-methyl-1-[2-(methyloxy)ethyl]-1H-benzimidazole-5-carboxamide,
2-[2-(1H-indazol-5-yl)-1H-imidazol-4-yl]-N-methyl-1-[2-(methyloxy)ethyl]-1H-benzimidazole-5-carboxamide,
2-[2-(1H-indazol-6-yl)-1H-imidazol-4-yl]-N-methyl-1-[2-(methyloxy)ethyl]-1H-benzimidazole-5-carboxamide,
2-[2-(2,1,3-benzoxadiazol-5-yl)-1H-imidazol-4-yl]-N-methyl-1-[2-(methyloxy)ethyl]-1H-benzimidazole-5-carboxamide,
2-[2-(2,1,3-benzoxadiazol-5-yl)-1H-imidazol-4-yl]-N,N-dimethyl-1-[2-(methyloxy)ethyl]-1H-benzimidazole-5-carboxamide,
2-[2-(1,3-benzothiazol-5-yl)-1H-imidazol-4-yl]-N,N-dimethyl-1-[2-(methyloxy)ethyl]-1H-benzimidazole-5-carboxamide,
5-(4-{6-methyl-1-[2-(methyloxy)ethyl]-1H-benzimidazol-2-yl}-1H-imidazol-2-yl)-2,1,3-benzoxadiazole,
5-(4-{6-methyl-1-[2-(methyloxy)ethyl]-1H-benzimidazol-2-yl}-1H-imidazol-2-yl)-1,3-benzothiazole,
2-{2-[4-chloro-3-(methyloxy)phenyl]-1H-imidazol-4-yl}-6-methyl-1-[2-(methyloxy)ethyl]-1H-benzimidazole,
2-chloro-5-(4-{6-methyl-1-[2-(methyloxy)ethyl]-1H-benzimidazol-2-yl}-1H-imidazol-2-yl)phenol,
2-[2-(4-chlorophenyl)-1H-imidazol-4-yl]-1-[2-(methyloxy)ethyl]-1H-benzimidazole, methyl 2-[2-(4-chlorophenyl)-1H-imidazol-4-yl]-1-[2-(methyloxy)ethyl]-1H-benzimidazole-5-carboxylate,
2-[2-(4-chlorophenyl)-1H-imidazol-4-yl]-N,1-bis[2-(methyloxy)ethyl]-1H-benzimidazole-5-carboxamide,
2-[2-(4-chlorophenyl)-1H-imidazol-4-yl]-N-methyl-1-[2-(methyloxy)ethyl]-1H-benzimidazole-5-carboxamide,
2-[2-(4-chlorophenyl)-1H-imidazol-4-yl]-N,N-dimethyl-1-[2-(methyloxy)ethyl]-1H-benzimidazole-5-carboxamide,
2-[2-(4-chlorophenyl)-1H-imidazol-4-yl]-1-[2-(methyloxy)ethyl]-5-(trifluoromethyl)-1H-benzimidazole,
2-[2-(4-chlorophenyl)-1H-imidazol-4-yl]-6-(methyloxy)-1-[2-(methyloxy)ethyl]-1H-benzimidazole,
2-[2-(4-chlorophenyl)-1H-imidazol-4-yl]-5-methyl-1-[2-(methyloxy)ethyl]-1H-benzimidazole,
2-[2-(4-chlorophenyl)-1H-imidazol-4-yl]-6-methyl-1-[2-(methyloxy)ethyl]-1H-benzimidazole,
N,1-bis[2-(methyloxy)ethyl]-2-(2-phenyl-1H-imidazol-4-yl)-1H-benzimidazole-5-carboxamide,
2-[2-(4-chlorophenyl)-1H-imidazol-4-yl]-1-(2-hydroxyethyl)-N-[2-(methyloxy)ethyl]-1H-benzimidazole-5-carboxamide,
2-[2-(4-chlorophenyl)-1H-imidazol-4-yl]N-methyl-1-[2-(methyloxy)ethyl]-1H-benzimidazole-6-carboxamide,
2-[2-(4-chlorophenyl)-1H-imidazol-4-yl]-1-[2-(methyloxy)ethyl]-1H-benzimidazole-6-carboxylic acid,
2-[2-(3-chlorophenyl)-1H-imidazol-4-yl]N-methyl-1-[2-(methyloxy)ethyl]-1H-benzimidazole-5-carboxamide,
2-[2-(4-chlorophenyl)-1H-imidazol-4-yl]-N-methyl-1-propyl-1H-benzimidazole-5-carboxamide,
2-[2-(4-chlorophenyl)-1H-imidazol-4-yl]-1-[2-(ethyloxy)ethyl]-N-methyl-1H-benzimidazole-5-carboxamide,
2-[2-(4-chlorophenyl)-1H-imidazol-4-yl]-7-methyl-1-[2-(methyloxy)ethyl]-1H-benzimidazole,
2-[2-(4-chlorophenyl)-1H-imidazol-4-yl]-1-[2-(methyloxy)ethyl]-N-4-pyridinyl-1H-benzimidazole-5-carboxamide,
2-[2-(4-chlorophenyl)-1H-imidazol-4-yl]-1-[2-(methyloxy)ethyl]-N-2-pyridinyl-1H-benzimidazole-6-carboxamide,
2-[2-(4-chlorophenyl)-1H-imidazol-4-yl]-1-[2-(methyloxy)ethyl]-N-3-pyridinyl-1H-benzimidazole-6-carboxamide,
2-[2-(4-chlorophenyl)-1H-imidazol-4-yl]-1-[2-(methyloxy)ethyl]-N-4-pyridinyl-1H-benzimidazole-6-carboxamide,
2-[2-(4-chlorophenyl)-1H-imidazol-4-yl]-1-[2-(methyloxy)ethyl]-N-2-pyridinyl-1H-benzimidazole-5-carboxamide,
2-[2-(4-chlorophenyl)-1H-imidazol-4-yl]-1-[2-(methyloxy)ethyl]-1H-imidazo[4,5-c]pyridine,
2-[2-(1,3-benzothiazol-5-yl)-1H-imidazol-4-yl]-1-[2-(methyloxy)ethyl]-1H-benzimidazole-6-carbonitrile,
5-{4-[6-methyl-1-[2-(methyloxy)ethyl]-5-(methylsulfonyl)-1H-benzimidazol-2-yl]-1H-imidazol-2-yl}-1,3-benzothiazole,
5-(4-{6-fluoro-1-[2-(methyloxy)ethyl]-1H-benzimidazol-2-yl}-1H-imidazol-2-yl)-1,3-benzothiazole,
5-(4-{7-chloro-1-[2-(methyloxy)ethyl]-1H-benzimidazol-2-yl}-1H-imidazol-2-yl)-1,3-benzothiazole,
5-(4-{4-methyl-1-[2-(methyloxy)ethyl]-1H-benzimidazol-2-yl}-1H-imidazol-2-yl)-1,3-benzothiazole,
5-(4-{6-chloro-1-[2-(methyloxy)ethyl]-1H-benzimidazol-2-yl}-1H-imidazol-2-yl)-1,3-benzothiazole,
5-(4-{6-bromo-1-[2-(methyloxy)ethyl]-1H-benzimidazol-2-yl}-1H-imidazol-2-yl)-1,3-benzothiazole,
5-(4-{6-(ethyloxy)-1-[2-(methyloxy)ethyl]-1H-benzimidazol-2-yl}-1H-imidazol-2-yl)-1,3-benzothiazole,
5-{4-[1-[2-(methyloxy)ethyl]-6-(trifluoromethyl)-1H-benzimidazol-2-yl]-1H-imidazol-2-yl}-1,3-benzothiazole,
2-[2-(1,3-benzothiazol-5-yl)-1H-imidazol-4-yl]-N,N,6-trimethyl-1-[2-(methyloxy)ethyl]-1H-benzimidazole-5-carboxamide,
5-{4-[6-methyl-1-[2-(methyloxy)ethyl]-5-(4-morpholinylcarbonyl)-1H-benzimidazol-2-yl]-1H-imidazol-2-yl}-1,3-benzothiazole,
5-(4-{6-methyl-5-{[(3R)-3-methyl-4-morpholinyl]carbonyl}-1-[2-(methyloxy)ethyl]-1H-benzimidazol-2-yl}-1H-imidazol-2-yl)-1,3-benzothiazole,
5-(4-{6-methyl-5-{[(3S)-3-methyl-4-morpholinyl]carbonyl}-1-[2-(methyloxy)ethyl]-1H-benzimidazol-2-yl}-1H-imidazol-2-yl)-1,3-benzothiazole,
5-(4-{6-methyl-1-[2-(methyloxy)ethyl]-5-[(4-methyl-1-piperazinyl)carbonyl]-1H-benzimidazol-2-yl}-1H-imidazol-2-yl)-1,3-benzothiazole,
methyl 2-[2-(1,3-benzothiazol-5-yl)-1H-imidazol-4-yl]-1-[2-(methyloxy)ethyl]-1H-benzimidazole-6-carboxylate,
2-[2-(1,3-benzothiazol-5-yl)-1H-imidazol-4-yl]-1-[2-(methyloxy)ethyl]-1H-benzimidazole-6-carboxylic acid,
methyl 2-[2-(1,3-benzothiazol-5-yl)-1H-imidazol-4-yl]-1-[2-(methyloxy)ethyl]-1H-benzimidazole-5-carboxylate,
2-{2-[2-(1,3-benzothiazol-5-yl)-1H-imidazol-4-yl]-1-[2-(methyloxy)ethyl]-1H-benzimidazol-5-yl}-2-propanol,
2-{2-[2-(1,3-benzothiazol-5-yl)-1H-imidazol-4-yl]-1-[2-(methyloxy)ethyl]-1H-benzimidazol-5-yl}-2-propanol,
2-[2-(1,3-benzothiazol-5-yl)-1H-imidazol-4-yl]-3-[2-(methyloxy)ethyl]-3H-imidazo[4,5-b]pyridine,
2-[2-(1,3-benzothiazol-5-yl)-1H-imidazol-4-yl]-5-methyl-3-[2-(methyloxy)ethyl]-3H-imidazo[4,5-b]pyridine,
2-[2-(1,3-benzothiazol-5-yl)-1H-imidazol-4-yl]-1-[2-(methyloxy)ethyl]-1H-imidazo[4,5-b]pyridine,
2-[2-(1,3-benzothiazol-5-yl)-1H-imidazol-4-yl]-6-methyl-3-[2-(methyloxy)ethyl]-3H-imidazo[4,5-b]pyridine,
{2-[2-(1,3-benzothiazol-5-yl)-1H-imidazol-4-yl]-1-[2-(methyloxy)ethyl]-1H-benzimidazol-5-yl}methanol,
5-{4-[1-[2-(methyloxy)ethyl]-5-(trifluoromethyl)-1H-benzimidazol-2-yl]-1H-imidazol-2-yl}-1,3-benzothiazole,
5-{4-[1-[2-(methyloxy)ethyl]-5-(methylsulfonyl)-1H-benzimidazol-2-yl]-1H-imidazol-2-yl}-1,3-benzothiazole,
2-[2-(1,3-benzothiazol-5-yl)-1H-imidazol-4-yl]-1-[2-(methyloxy)ethyl]-1H-benzimidazole-5-sulfonamide,
5-(4-{5-bromo-1-[2-(methyloxy)ethyl]-1H-benzimidazol-2-yl}-1H-imidazol-2-yl)-1,3-benzothiazole,
2-[2-(1,3-benzothiazol-5-yl)-1H-imidazol-4-yl]-1-[2-(methyloxy)ethyl]-1H-benzimidazole-5-carbonitrile,
2-[2-(1,3-benzothiazol-5-yl)-1H-imidazol-4-yl]-6-chloro-1-[2-(methyloxy)ethyl]-1H-benzimidazole-5-carboxylic acid,
5-(4-{1-[2-(methyloxy)ethyl]-1H-benzimidazol-2-yl}-1H-imidazol-2-yl)-1,3-benzothiazole,
5-{4-[1-[2-(methyloxy)ethyl]-4-(methylsulfonyl)-1H-benzimidazol-2-yl]-1H-imidazol-2-yl}-1,3-benzothiazole,
5-(4-{1-[2-(methyloxy)ethyl]-4-[(phenylmethyl)sulfonyl]-1H-benzimidazol-2-yl}-1H-imidazol-2-yl)-1,3-benzothiazole,
5-{4-[1-[2-(methyloxy)ethyl]-4-(4-morpholinyl)-1H-benzimidazol-2-yl]-1H-imidazol-2-yl}-1,3-benzothiazole,
2-[2-(1,3-benzothiazol-5-yl)-1H-imidazol-4-yl]-1-[2-(methyloxy)ethyl]-4-(4-morpholinyl)-1H-imidazo[4,5-c]pyridine,
2-[2-(1,3-benzothiazol-5-yl)-1H-imidazol-4-yl]-4-[(3S)-3-methyl-4-morpholinyl]-1-[2-(methyloxy)ethyl]-1H-imidazo[4,5-c]pyridine,
2-[2-(1,3-benzothiazol-5-yl)-1H-imidazol-4-yl]-4-[(3R)-3-methyl-4-morpholinyl]-1-[2-(methyloxy)ethyl]-1H-imidazo[4,5-c]pyridine,
2-[2-(1,3-benzothiazol-5-yl)-1H-imidazol-4-yl]-4-[(3R)-3-ethyl-4-morpholinyl]-1-[2-(methyloxy)ethyl]-1H-imidazo[4,5-c]pyridine,
2-[2-(1,3-benzothiazol-5-yl)-1H-imidazol-4-yl]-4-(2-methyl-4-morpholinyl)-1-[2-(methyloxy)ethyl]-1H-imidazo[4,5-c]pyridine,
((2S,5R)-4-{2-[2-(1,3-benzothiazol-5-yl)-1H-imidazol-4-yl]-1-[2-(methyloxy)ethyl]-1H-imidazo[4,5-c]pyridin-4-yl}-5-ethyl-2-morpholinyl)methanol,
((2S,5S)-4-{2-[2-(1,3-benzothiazol-5-yl)-1H-imidazol-4-yl]-1-[2-(methyloxy)ethyl]-1H-imidazo[4,5-c]pyridin-4-yl}-5-methyl-2-morpholinyl)methanol,
2-[2-(1,3-benzothiazol-5-yl)-1H-imidazol-4-yl]-1-[2-(methyloxy)ethyl]-4-(1-piperidinyl)-1H-imidazo[4,5-c]pyridine,
2-[2-(1,3-benzothiazol-5-yl)-1H-imidazol-4-yl]-1-[2-(methyloxy)ethyl]-4-(1-pyrrolidinyl)-1H-imidazo[4,5-c]pyridine,
4-(1-azetidinyl)-2-[2-(1,3-benzothiazol-5-yl)-1H-imidazol-4-yl]-1-[2-(methyloxy)ethyl]-1H-imidazo[4,5-c]pyridine,
2-[2-(1,3-benzothiazol-5-yl)-1H-imidazol-4-yl]-1-[2-(methyloxy)ethyl]-4-(4-methyl-1-piperazinyl)-1H-imidazo[4,5-c]pyridine,
2-[2-(1,3-benzothiazol-5-yl)-1H-imidazol-4-yl]-4-[(3S)-3,4-dimethyl-1-piperazinyl]-1-[2-(methyloxy)ethyl]-1H-imidazo[4,5-c]pyridine,
2-[2-(1,3-benzothiazol-5-yl)-1H-imidazol-4-yl]-N-methyl-N,1-bis[2-(methyloxy)ethyl]-1H-imidazo[4,5-c]pyridin-4-amine,
2-[{2-[2-(1,3-benzothiazol-5-yl)-1H-imidazol-4-yl]-1-[2-(methyloxy)ethyl]-1H-imidazo[4,5-c]pyridin-4-yl}(methyl)amino]ethanol,
2-[2-(1,3-benzothiazol-5-yl)-1H-imidazol-4-yl]-N-ethyl-N,1-bis[2-(methyloxy)ethyl]-1H-imidazo[4,5-c]pyridin-4-amine,
2-[2-(1,3-benzothiazol-5-yl)-1H-imidazol-4-yl]-6-methyl-1-[2-(methyloxy)ethyl]-4-(4-morpholinyl)-1H-imidazo[4,5-c]pyridine,
5-{4-[1-[2-(methyloxy)ethyl]-5-(4-morpholinylsulfonyl)-1H-benzimidazol-2-yl]-1H-imidazol-2-yl}-1,3-benzothiazole,
5-{4-[1-[2-(methyloxy)ethyl]-5-(1-piperazinylsulfonyl)-1H-benzimidazol-2-yl]-1H-imidazol-2-yl}-1,3-benzothiazole,
5-{4-[1-[2-(methyloxy)ethyl]-5-(1-pyrrolidinylsulfonyl)-1H-benzimidazol-2-yl]-1H-imidazol-2-yl}-1,3-benzothiazole,
2-[2-(1,3-benzothiazol-5-yl)-1H-imidazol-4-yl]-N,1-bis[2-(methyloxy)ethyl]-1H-benzimidazole-5-sulfonamide,
2-[2-(1,3-benzothiazol-5-yl)-1H-imidazol-4-yl]-6-methyl-N,1-bis[2-(methyloxy)ethyl]-1H-benzimidazole-5-sulfonamide,
2-[2-(1,3-benzothiazol-5-yl)-1H-imidazol-4-yl]-6-methyl-1-[2-(methyloxy)ethyl]-1H-benzimidazole-5-carboxylic acid,
5-(4-{6-methyl-1-[2-(methyloxy)ethyl]-5-[(4-methyl-1-piperazinyl)sulfonyl]-1H-benzimidazol-2-yl}-1H-imidazol-2-yl)-1,3-benzothiazole,
5-{4-[6-methyl-1-[2-(methyloxy)ethyl]-5-(4-morpholinylsulfonyl)-1H-benzimidazol-2-yl]-1H-imidazol-2-yl}-1,3-benzothiazole,
2-[2-(1,3-benzothiazol-5-yl)-1H-imidazol-4-yl]-1-[2-(methyloxy)ethyl]-1H-benzimidazole-5-carboxylic acid,
5-(1-methyl-4-{6-methyl-1-[2-(methyloxy)ethyl]-1H-benzimidazol-2-yl}-1H-imidazol-2-yl)-1,3-benzothiazole,
2′-(1,3-benzothiazol-5-yl)-1-[2-(methyloxy)ethyl]-1H, 1′H-2,4′-biimidazole,
2′-(1,3-benzothiazol-5-yl)-4,5-dimethyl-1-[2-(methyloxy)ethyl]-1H,1′H-2,4′-biimidazole,
2′-(1,3-benzothiazol-5-yl)-4,5-diethyl-1-[2-(methyloxy)ethyl]-1H,1′H-2,4′-biimidazole, and
5-(4-{1-[2-(methyloxy)ethyl]-4,5,6,7-tetrahydro-1H-benzimidazol-2-yl}-1H-imidazol-2-yl)-1,3-benzothiazole,

in free base form, or in the form of a salt, particularly a pharmaceutically acceptable salt, thereof.

Representative compounds of this invention include the compounds of Examples 1-116.

Compound names were generated using the software naming program ACD/Name Pro V6.02 available from Advanced Chemistry Development, Inc., 110 Yonge Street, 14^{th} Floor, Toronto, Ontario, Canada, M5C 1T4 (http://www.acdlabs.com/). It will be appreciated by those skilled in the art that many of the compounds of this invention, as well as compounds used in the preparation of the compounds of Formula (I), (I-A), (I-B), (I-C), (II) or (III), may exist in tautomeric forms. The program used to name the compounds of this invention will only name one of such tautomeric forms at a time. It is to be understood that any reference to a named compound or a structurally depicted compound is intended to encompass all tautomers of such compounds and any mixtures of tautomers thereof.

The compounds according to Formula (I), (I-A), (I-B), (I-C), (II) or (III) may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of this invention, or in any chemical structure illustrated herein, is not specified the structure is intended to encompass all individual stereoisomers and all mixtures thereof. Thus, compounds according to Formula (I), (I-A), (I-B), (I-C), (II) or (III) containing one or more chiral center may be used as racemic mixtures, enantiomerically enriched mixtures, or as enantiomerically pure individual stereoisomers.

Individual stereoisomers of a compound according to according to Formula (I), (I-A), (I-B), (I-C), (II) or (III) which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation. When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

Because of their potential use in medicine, the salts of the compounds of Formula (I), (I-A), (I-B), (I-C), (II) or (III) are preferably pharmaceutically acceptable salts. Suitable pharmaceutically acceptable salts include those described by Berge, Bighley and Monkhouse J. Pharm. Sci (1977) 66, pp 1-19. Salts encompassed within the term “pharmaceutically acceptable salts” refer to non-toxic salts of the compounds of this invention.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, and the like, or with a pyranosidyl acid, such as glucuronic acid or galacturonic acid, or with an alpha-hydroxy acid, such as citric acid or tartaric acid, or with an amino acid, such as aspartic acid or glutamic acid, or with an aromatic acid, such as benzoic acid or cinnamic acid, or with a sulfonic acid, such as p-toluenesulfonic acid, methanesulfonic acid, ethanesulfonic acid or the like.

Suitable addition salts are formed from acids which form non-toxic salts and examples include acetate, p-aminobenzoate, ascorbate, aspartate, benzenesulfonate, benzoate, bicarbonate, bismethylenesalicylate, bisulfate, bitartrate, borate, calcium edetate, camsylate, carbonate, clavulanate, citrate, cyclohexylsulfamate, edetate, edisylate, estolate, esylate, ethanedisulfonate, ethanesulfonate, formate, fumarate, gluceptate, gluconate, glutamate, glycollate, glycollylarsanilate, hexylresorcinate, hydrabamine, hydrobromide, hydrochloride, dihydrochloride, hydrofumarate, hydrogen phosphate, hydroiodide, hydromaleate, hydrosuccinate, hydroxynaphthoate, isethionate, itaconate, lactate, lactobionate, laurate, malate, maleate, mandelate, mesylate, methylsulfate, monopotassium maleate, mucate, napsylate, nitrate, N-methylglucamine, oxalate, oxaloacetate, pamoate (embonate), palmate, palmitate, pantothenate, phosphate/diphosphate, pyruvate, polygalacturonate, propionate, saccharate, salicylate, stearate, subacetate, succinate, sulfate, tannate, tartrate, teoclate, tosylate, triethiodide, trifluoroacetate and valerate.

Other exemplary acid addition salts include pyrosulfate, sulfite, bisulfite, decanoate, caprylate, acrylate, isobutyrate, caproate, heptanoate, propiolate, oxalate, malonate, suberate, sebacate, butyne-1,4-dioate, hexyne-1,6-dioate, chlorobenzoate, methylbenzoate, dinitrobenzoate, hydroxybenzoate, methoxybenzoate, phthalate, phenylacetate, phenylpropionate, phenylbutrate, lactate, γ-hydroxybutyrate, mandelate, and sulfonates, such as xylenesulfonate, propanesulfonate, naphthalene-1-sulfonate and naphthalene-2-sulfonate.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as N-methyl-D-glucamine, diethylamine, isopropylamine, trimethylamine, ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

Compounds of the invention having both a basic and acidic moiety may be in the form of zwitterions, acid-addition salt of the basic moiety or base salts of the acidic moiety. This invention also provides for the conversion of one pharmaceutically acceptable salt of a compound of this invention, e.g., a hydrochloride salt, into another pharmaceutically acceptable salt of a compound of this invention, e.g., a sodium salt.

For solvates of the compounds of the invention, or salts thereof that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The subject invention also includes isotopically-labeled compounds which are identical to those recited in according to Formula (I), (I-A), (I-B), (I-C), (II) or (III) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography).

Because the compounds of Formula (I), (I-A), (I-B), (I-C), (II) or (III) are intended for use in pharmaceutical compositions it will readily be understood that they are each preferably provided in substantially pure form, for example at least 60% pure, more suitably at least 75% pure and preferably at least 85%, especially at least 98% pure (')/0 are on a weight for weight basis). Impure preparations of the compounds may be used for preparing the more pure forms used in the pharmaceutical compositions.

### General Synthetic Methods

The compounds of Formula (I), (I-A), (I-B), (I-C), (II) or (III) may be obtained by using synthetic procedures illustrated in the Schemes below or by drawing on the knowledge of a skilled organic chemist. The synthesis provided in these Schemes are applicable for producing compounds of the invention having a variety of different R¹ and R² groups employing appropriate precursors, which are suitably protected if needed, to achieve compatibility with the reactions outlined herein. Subsequent deprotection, where needed, affords compounds of the nature generally disclosed. While the Schemes are shown with compounds only of Formula (I), (I-A), (I-B), (I-C), (II) or (III), they are illustrative of processes that may be used to make the compounds of the invention.

Intermediates (compounds used in the preparation of the compounds of the invention) may also be present as salts. Thus, in reference to intermediates, the phrase “compound(s) of formula (number)” means a compound having that structural formula or a pharmaceutically acceptable salt thereof.

Examples 1-5 (Table 1) can be made as illustrated in scheme 1. 4-Fluoro-3-nitrobenzoic acid 1.1 was treated with oxalyl chloride in an appropriate solvent followed by addition of an amine to afford amide 1.2. Amide 1.2 was then heated with an amine in the presence of Hunig's base in EtOH to produce nitro-aniline 1.3 which was subsequently reduced to dianiline 1.4 using either zinc ammonium formate or zinc acetic acid. The benzimidazole final product (1-9) was obtained by condensation and cyclization of dianiline 1.4 with 2-(4-chlorophenyl)-1H-imidazole-4-carbaldehyde in the presence of sodium bisulfite.

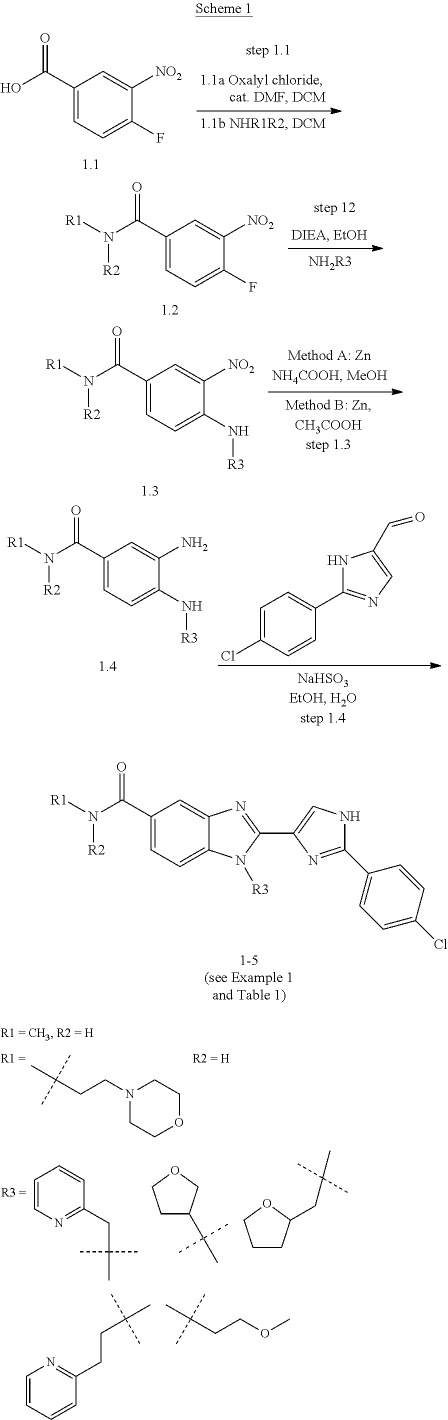

Scheme 2 showed how imidazole aldehyde used in scheme 1 was generally made. Cyano compounds (2.1) were converted to amidines (2.2) then treated with 2,5-bis(hydroxymethyl)-1,4-dioxane-2,5-diol (DHAD) to afford imidazole alcohol 2.3, which was subsequently oxidized to aldehyde 2.4. The aldehyde 2.4 was condensed and cyclized with 1.4 to give rise to benzimidazole 8-12.

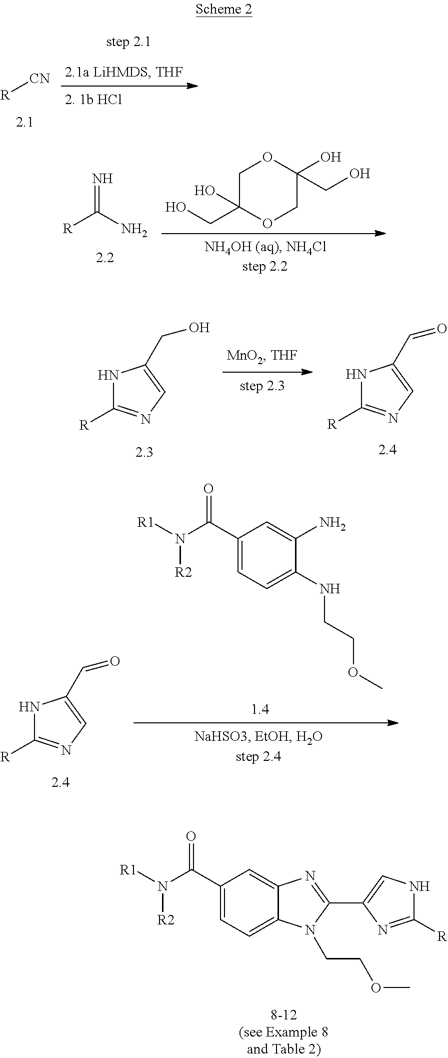

Alternatively, benzimidazoles 13-31 can be made as shown in scheme 3. Protection of imidazole 3.1 with SEM-Cl, followed by bromination using NBS and AlBN to afford bromoimidazole 3.3. Ester 3.3 was reduced to a mixture of aldehyde 3.4 along with alcohol 3.5 which can be converted to aldehyde 3.4 using MnO₂ later. The aldehyde 3.4 was then condensed and cyclized with either 1.4 (scheme 1) or 3.10 to afford formulae 3.6, followed by Suzuki coupling with boronic acid or ester and deprotection of imidazole to provide compounds 13 and 15-30. Formulae 3.7 can also be converted to compounds 14 and 31 using BBr₃.

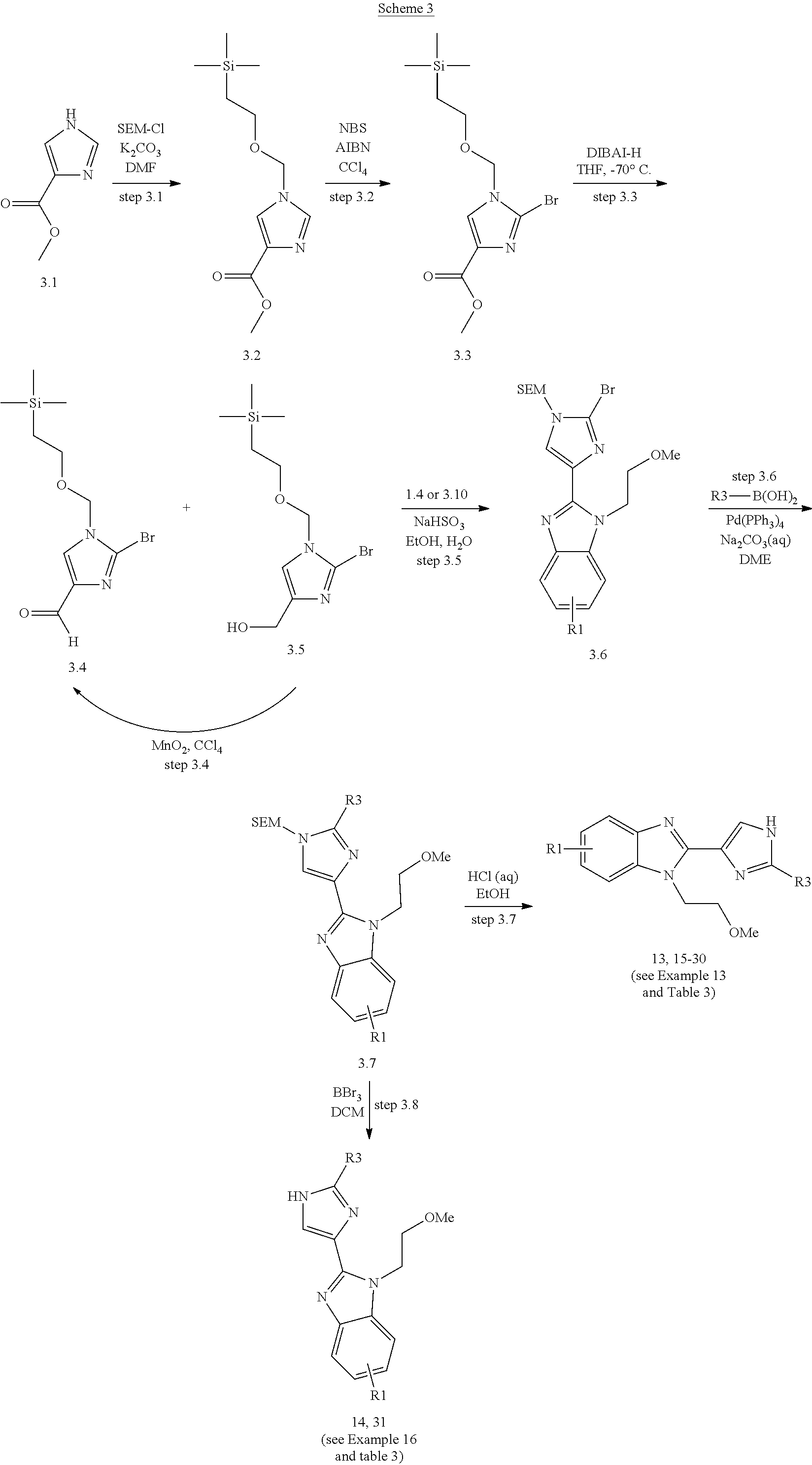

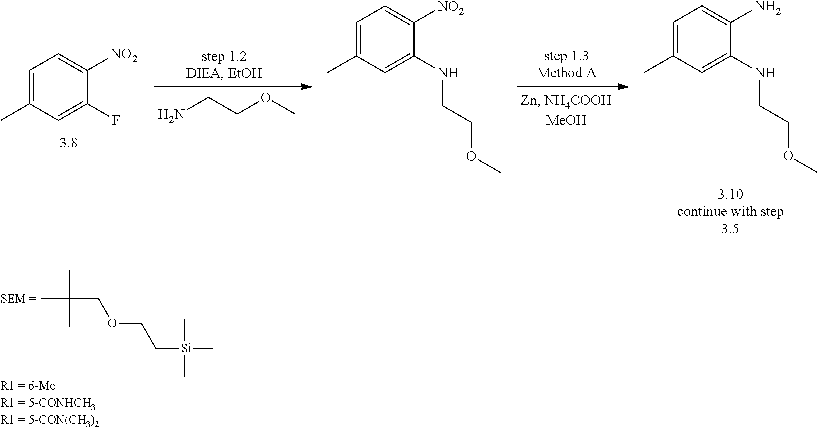

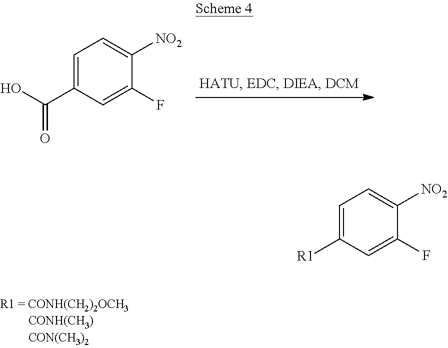

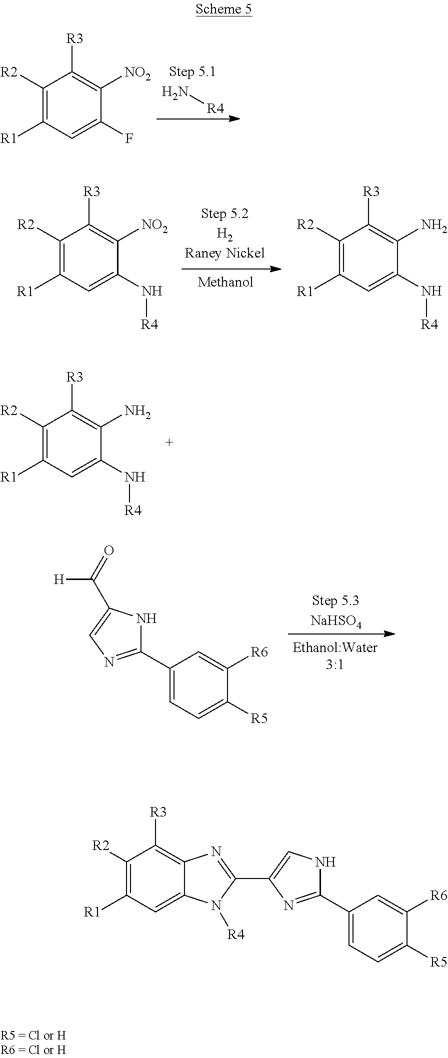

| R₁ | R₂ | R₃ | R₄ |
|---|---|---|---|
| COOCH₃ | H | H | (CH₂)₂OCH₃ |
| H | H | H | (CH₂)₂OCH₃ |
| CON(CH₂)₂OCH₃ | H | H | (CH₂)₂OCH₃ |
| CON(CH₂)₂OCH₃ | H | H | (CH₂)₂OH |
| CON(CH₃) | H | H | (CH₂)₂OCH₃ |
| CON(CH₃) | H | H | (CH₂)₂CH₃ |
| CON(CH₃) | H | H | (CH₂)₂O CH₂CH₃ |
| CON(CH₃) | H | H | CH₂CCH₂ |
| CON(CH₃)₂ | H | H | (CH₂)₂OCH₃ |
| CF₃ | H | H | (CH₂)₂OCH₃ |
| CH₃ | H | H | (CH₂)₂OCH₃ |
| COOH | H | H | (CH₂)₂OCH₃ |
| CON(CH₃) | H | H | CH₃ |
| H | Cl | H | (CH₂)₂OCH₃ |
| H | OCH₃ | H | (CH₂)₂OCH₃ |
| H | CH₃ | H | (CH₂)₂OCH₃ |
| H | CON(CH₃) | H | (CH₂)₂OCH₃ |
| H | COOH | H | (CH₂)₂OCH₃ |
| H | CH₃ | H | CH₃ |
| H | H | CH₃ | (CH₂)₂OCH₃ |
| H | H | H | H |

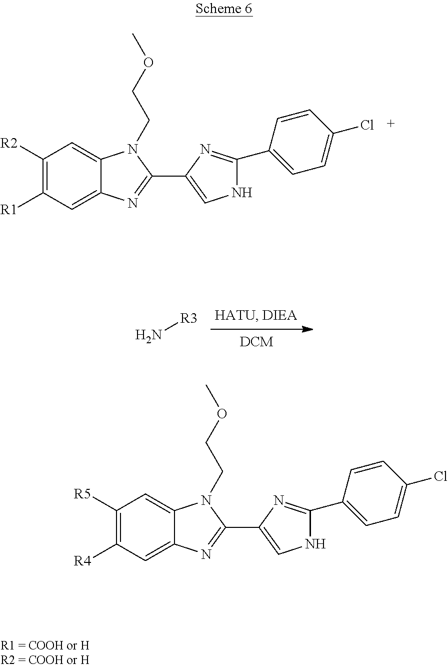

The present invention is also directed to a method of inhibiting RIP2 kinase which comprises contacting the kinase with a compound according to Formula (I), (I-A), (I-B), (I-C), (II) or (III), or a salt, particularly a pharmaceutically acceptable salt, thereof. This invention is also directed to a method of treatment of a RIP2-mediated disease or disorder comprising administering a therapeutically effective amount of a compound according to Formula (I), (I-A), (I-B), (I-C), (II) or (III), or a salt thereof, particularly a pharmaceutically acceptable salt thereof, to a patient, specifically a human, in need thereof. As used herein, “patient” refers to a human or other mammal. The invention is still further directed to the use of a compound according to Formula (I), (I-A), (I-B), (I-C), (II) or (III), or a salt thereof, particularly a pharmaceutically acceptable salt thereof, or a pharmaceutical composition comprising a compound according to Formula (I), (I-A), (I-B), (I-C), (II) or (III), or a salt thereof, particularly a pharmaceutically acceptable salt thereof, to inhibit RIP2 kinase and/or treat a RIP2 kinase-mediated disease or disorder.

The compounds of this invention may be particularly useful for treatment of the following RIP2-mediated diseases or disorders, particularly, uveitis, interleukin-1 converting enzyme (ICE, also known as Caspase-1) associated fever syndrome, dermatitis, type 2 diabetes mellitus, acute lung injury, arthritis (specifically rheumatoid arthritis), inflammatory bowel disorders (such as ulcerative colitis and Crohn's disease), prevention of ischemia reperfusion injury in solid organ transplant, liver diseases (non-alcohol steatohepatitis, alcohol steatohepatitis, autoimmune hepatitis), allergic diseases (such as asthma), autoimmune diseases (such as systemic lupus erythematosus and Multiple Sclerosis), transplant reactions (such as graft versus host disease) and granulomateous disorders, such as adult sarcoidosis, Blau syndrome, early-onset sarcoidosis, cutaneous sarcoidosis, Wegner's granulomatosis, and interstitial pulmonary disease. The compounds of this invention may be particularly useful in the treatment of uveitis, ICE fever, Blau Syndrome/early-onset sarcoidosis, ulcerative colitis, Crohn's disease, Wegener's granulamatosis and sarcoidosis.

Treatment of RIP2-mediated disease conditions, or more broadly, treatment of immune mediated disease, such as, but not limited to, allergic diseases, autoimmune diseases, prevention of transplant rejection and the like, may be achieved using a compound of this invention of as a monotherapy, or in dual or multiple combination therapy, particularly for the treatment of refractory cases, such as in combination with other anti-inflammatory and/or anti-TNF agents, which may be administered in therapeutically effective amounts as is known in the art. For example, the compounds of this invention may be administered in combination with corticosteroids and/or anti-TNF agents to treat Blau syndrome/early-onset sarcoidosis; or in combination with anti-TNF biologics or other anti-inflammatory biologics to treat Crohn's Disease; or in combination with low-dose corticosteroids and/or methotrexate to treat Wegener's granulamatosis or sarcoidosis or interstitial pulmonary disease; or in combination with a biologic (e.g. anti-TNF, anti-IL-6, etc.) to treat rheumatoid arthritis; or in combination with anti-IL6 and or methotrexate to treat ICE fever.

Examples of suitable anti-inflammatory agents include corticosteroids, particularly low-dose corticosteroids (such as Deltasone® (prednisone)) and anti-inflammatory biologics (such as Acterma® (anti-IL6R mAb) and Rituximab® (anti-CD20 mAb)). Examples of suitable anti-TNF agents include anti-TNF biologics (such as Enbrel® (etanecerpt)), Humira® (adalimumab), Remicade® (infliximab) and Simponi® (golimumab)).

This invention also provides a compound according to Formula (I), (I-A), (I-B), (I-C), (II) or (III), or a salt thereof, particularly a pharmaceutically acceptable salt thereof, for use in the treatment or prophylaxis of RIP2-mediated diseases or disorders, for example those diseases and disorders mentioned hereinabove.

The invention also provides the use of a compound according to Formula (I), (I-A), (I-B), (I-C), (II) or (III), or a salt thereof, particularly a pharmaceutically acceptable salt thereof, in the manufacture of a medicament for the treatment or prophylaxis of RIP2-mediated diseases or disorders, for example those diseases and disorders mentioned hereinabove. Accordingly, the present invention is also directed to pharmaceutical compositions comprising a compound according to Formula (I), (I-A), (I-B), (I-C), (II) or (III), or a salt thereof, particularly a pharmaceutically acceptable salt thereof.

A therapeutically “effective amount” is intended to mean that amount of a compound that, when administered to a patient in need of such treatment, is sufficient to effect treatment, as defined herein. Thus, e.g., a therapeutically effective amount of a compound of Formula (I), (I-A), (I-B), (I-C), (II) or (III), or a pharmaceutically acceptable salt thereof, is a quantity of an inventive agent that, when administered to a human in need thereof, is sufficient to modulate or inhibit the activity of RIP2 kinase such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmaceutical characteristics), disease or condition and its severity and the specific composition and method being used, but can nevertheless be determined by one of skill in the art.

“Treating” or “treatment” is intended to mean at least the mitigation of a disease condition in a patient. The methods of treatment for mitigation of a disease condition include the use of the compounds in this invention in any conventionally acceptable manner, for example for prevention, retardation, prophylaxis, therapy or cure of a mediated disease. Specific diseases and conditions that may be particularly susceptible to treatment using a compound of this invention are described herein.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

For use in therapy, the compounds of the invention will be normally, but not necessarily, formulated into a pharmaceutical composition prior to administration to a patient. Accordingly, the invention is also directed to pharmaceutical compositions comprising a compound of the invention and a pharmaceutically-acceptable excipient.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula (I), (I-A), (I-B), (I-C), (II) or (III) or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

The compounds of the invention and the pharmaceutically-acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Names for the intermediate and final compounds described herein were generated using a commercially available software naming program. It will be appreciated by those skilled in the art that in certain instances such programs will name a structurally depicted compound (e.g., intermediates of Preparation 23) as a single tautomer of that compound. It is to be understood that any reference to a named compound or a structurally depicted compound is intended to encompass all tautomers of such compounds and any mixtures of tautomers thereof.

In the following experimental descriptions, the following abbreviations may be used:

| Abbreviation | Meaning |
|---|---|
| AcOH | acetic acid |
| aq | aqueous |
| brine | saturated aqueous NaCl |
| CH₂Cl₂ | methylene chloride |
| CH₃CN or MeCN | acetonitrile |
| CH₃NH₂ | methylamine |
| d | day |
| DMF | N,N-dimethylformamide |
| DMSO | dimethylsulfoxide |
| equiv | equivalents |
| Et | ethyl |
| Et₃N | triethylamine |
| Et₂O | diethyl ether |
| EtOAc | ethyl acetate |
| h, hr | hour |
| HCl | hydrochloric acid |
| i-Pr₂NEt | N′,N′-diisopropylethylamine |
| KOt-Bu | potassium tert-butoxide |
| LCMS | liquid chromatography-mass spectroscopy |
| Me | methyl |
| MeOH or CH₃OH | methanol |
| MgSO₄ | magnesium sulfate |
| min | minute |
| MS | mass spectrum |
| μw | microwave |
| NaBH₄ | sodium borohydride |
| Na₂CO₃ | sodium carbonate |
| NaHCO₃ | sodium bicarbonate |
| NaOH | sodium hydroxide |
| Na₂SO₄ | sodium sulfate |
| NH₄Cl | ammonium chloride |
| NiCl₂•6H₂O | nickel (II) chloride hexahydrate |
| NMP | N-methyl-2-pyrrolidone |
| Ph | phenyl |
| rt | room temperature |
| satd | saturated |
| SCX | strong cation exchange |
| SPE | solid phase extraction |
| TFA | trifluoroacetic acid |
| THF | tetrahydrofuran |
| t_{R} | retention time |

### Preparation 1

### 4-fluoro-N-methyl-3-nitrobenzamide

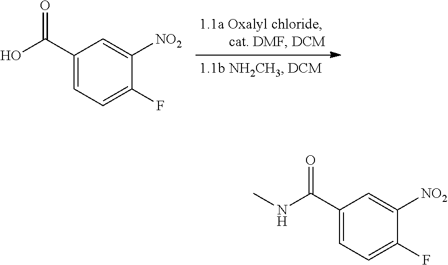

Oxalyl chloride (7.09 mL, 81 mmol) was dissolved in dichloromethane (DCM) (100 mL) under nitrogen and cooled in an ice/brine bath. N,N-dimethylformamide (DMF) (0.42 mL, 5.4 mmol) was added dropwise and the mixture was stirred cooled for 10 minutes. Then 4-fluoro-3-nitrobenzoic acid (10.0 g, 54.0 mmol) was added slowly and reaction mixture was stirred cooled for 30 minutes. Methyl amine solution (135 mL, 270 mmol, 2M in THF) was added and reaction mixture was stirred at room temperature overnight. Reaction was diluted with satd. NaHCO₃ and more DCM, layers were separated and organics were dried over sodium sulfate. Organics were concentrated to give a solid which was triturated in diethyl ether, filtered and dried to give the title compound as a bright orange/yellow solid (9.72 g, 82%, >90% pure). ¹H NMR (400 MHz, CHLOROFORM-d) δ 8.48 (dd, J=2.27, 7.07 Hz, 1H), 8.15 (m, 1H), 7.41 (dd, J=8.72, 10.23 Hz, 1H), 6.34 (br. s., 1H), 3.07 (d, J=3.79 Hz, 3H); MS (m/z) 198.9 W.

### Preparation 2

### N-methyl-4-{[2-(methyloxy)ethyl]amino}-3-nitrobenzamide

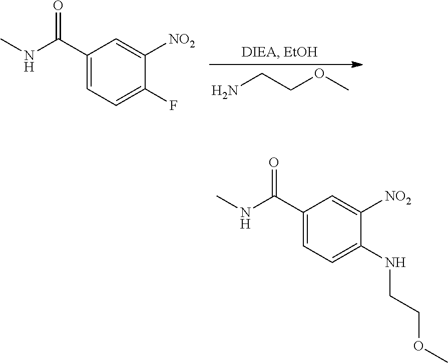

The 4-fluoro-N-methyl-3-nitrobenzamide (2.63 g, 13.3 mmol) was partially dissolved in ethanol (7 mL) in a microwave vial and N,N-diisopropylethylamine (DIEA) (2.3 mL, 13.3 mmol) and [2-(methyloxy)ethyl]amine (1.16 mL, 13.3 mmol) were added. The vial was capped and heated in the microwave at 150° C. for 15 minutes. Reaction was repeated two times on same scale and all crude reactions were combined. Mixture was cooled to room temperature and left to sit for 2 hours. A solid formed and it was filtered, rinsed with diethyl ether and dried to give the title compound as an orange solid (9.19 g, 86%, >95% purity). Product was taken on without further purification. ¹H NMR (400 MHz, DMSO-d₆) δ 8.63 (d, J=2.27 Hz, 1H), 8.48 (d, J=4.55 Hz, 1H), 8.36-8.44 (m, 1H), 7.99 (dd, J=2.02, 9.09 Hz, 1H), 7.16 (d, J=9.09 Hz, 1H), 3.51-3.69 (m, 4H), 3.32 (s, 3H), 2.77 (d, J=4.55 Hz, 3H); MS (m/z) 254.0 (M+H)⁺.

### Preparation 3

### 4-fluoro-N-[2-(methyloxy)ethyl]-3-nitrobenzamide

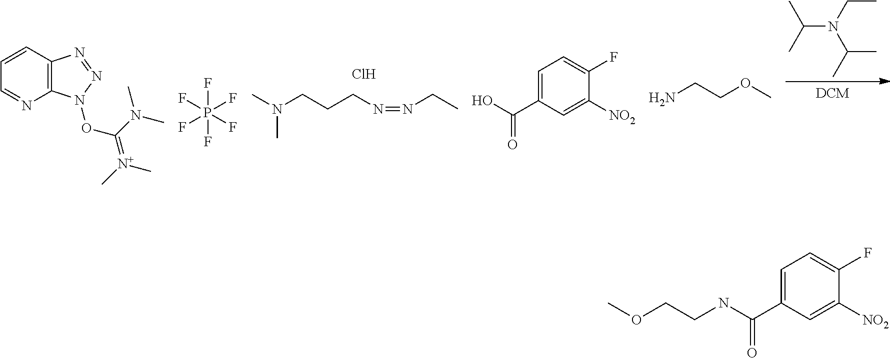

4-Fluoro-3-nitrobenzoic acid (2 g, 10.80 mmol) was dissolved in Dichloromethane (DCM) (30 mL) and to that was added HATU (6.16 g, 16.21 mmol), EDC (3.11 g, 16.21 mmol), and DIEA (3.77 mL, 21.61 mmol). Once everything was in solution, 2-(methyloxy)ethanamine (1.055 g, 14.05 mmol) was added and the reaction was allowed to stir until complete by LCMS. Organic phase was washed with saturated bicarbonate twice and then brine twice. The organic layer was dried over sodium sulphate, filtered and then removed under vacuum. 100% recovery was assumed and carried onto next reaction without further purification.

### Preparation 4

### 4-Fluoro-2-methyl-5-nitrobenzenesulfonyl chloride

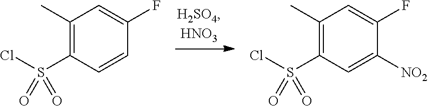

A mixture of 4-fluoro-2-methylbenzenesulfonyl chloride (3.8 g, 18.21 mmol) and sulfuric acid (13 mL) was cooled to 0° C. and nitric acid (1.5 mL, 60-70%) was added to the solution in ca. 5 min. The reaction was stirred for 1 hr, then diluted with EtOAc and poured into ice-water. The aqueous was extracted with EtOAc (2×80 mL), extracts were combined, washed with brine, dried (MgSO4), concentrated to give 4-fluoro-2-methyl-5-nitrobenzenesulfonyl chloride (4.5 g, 17.74 mmol, 97% yield) as orange oil. The ¹HNMR is identical as reported in the literature (WO 2009/069311 A1). ¹H NMR (400 MHz, CHLOROFORM-d) 8.8-8.9 (d, 1H); 7.38-7.45 (d, 1H), 2.9 (s., 3H).

### Example 1

### 2-[2-(4-chlorophenyl)-1H-imidazol-4-yl]-1-[2-(methyloxy)ethyl]-N-[2-(4-morpholinyl)ethyl]-1H-benzimidazole-5-carboxamide

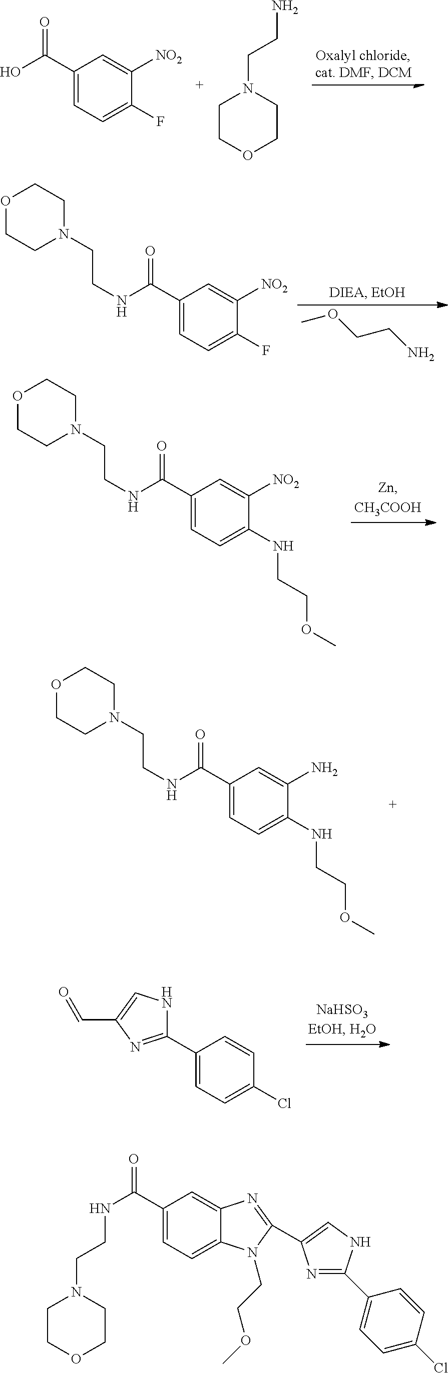

Step 1. 4-fluoro-N-[2-(4-morpholinyl)ethyl]-3-nitrobenzamide: Oxalyl chloride (0.34 ml, 4.05 mmol) was dissolved in DCM (10 ml) under nitrogen and cooled in an ice/brine bath. DMF was added dropwise to the above solution and the reaction was stirred cooled for 10 minutes. Then 4-fluoro-3-nitrobenzoic acid (500 mg, 2.7 mmol) was added and the reaction was stirred cooled for 30 minutes. The mixture was concentrated on rotovap and redissolved in DCM. Then add TEA (turns orange) and [2-(4-morpholinyl)ethyl]amine (387 μl, 2.97 mmol) and the reaction was stirred at room temperature for 15 minutes. Reaction was diluted with saturated NaHCO₃ and more DCM, layers were separated and organic was dried over sodium sulfate. Concentrate to give 900 mg (95%) desired product as orange solid. MS (m/z) 298.0 (M+H)⁺.

Step 2. 4-{[2-(methyloxy)ethyl]amino}-N-[2-(4-morpholinyl)ethyl]-3-nitrobenzamide: 4-fluoro-N-[2-(4-morpholinyl)ethyl]-3-nitrobenzamide (900 mg, 2.57 mmol) was partially dissolved in ethanol in a microwave vial and DIEA (0.49 ml, 2.57 mmol) and [2-(methyloxy)ethyl]amine (0.23 ml, 2.57 mmol) were added. The vial was capped and heated in the microwave at 150° C. for 5 minutes. Complete by LCMS. Reaction was concentrated and purified by Biotage using 1-5% MeOH/DCM gradient to afford 963 mg (100%) yellow/orange solid which was carried to next step directly. MS (m/z) 353.1 (M+H)⁺.

Step 3. 4-{[2-(methyloxy)ethyl]amino}-N-[2-(4-morpholinyl)ethyl]-3-nitrobenzamide: 4-{[2-(methyloxy)ethyl]amino}-N-[2-(4-morpholinyl)ethyl]-3-nitrobenzamide (960 mg, 2.59 mmol) was dissolved in acetic acid (10 mL) and zinc (846 mg, 12.9 mmol) was added. The reaction was stirred at room temperature for 0.5 hours. The reaction was filtered through a large acrodisc and the filtrate was concentrated and partitioned between satd. NaHCO₃ and DCM. Aqueous was extracted several times with DCM. Organics were combined, concentrated and purified by Biotage (25 g silica column, 0.5-5% MeOH/DCM (plus NH₄OH), 20 min.; 5%, 10 min.) to give the title compound as a purple foam (331 mg, 39%). ¹H NMR (400 MHz, CHLOROFORM-d) δ (7.27, s, 1H), 7.24 (dd, J=2.02, 8.08 Hz, 1H), 6.70 (br. s., 1H), 6.64 (d, J=8.34 Hz, 1H), 3.72-3.82 (m, 4H), 3.69 (t, J=5.18 Hz, 2H), 3.57 (q, J=5.39 Hz, 2H), 3.43 (s, 3H), 3.36 (t, J=5.18 Hz, 2H), 2.64 (t, J=5.94 Hz, 2H), 2.56 (br. s., 4H); MS (m/z) 323.1 (M+H)⁺.

Step 4. 2-[2-(4-chlorophenyl)-1H-imidazol-4-yl]-1-[2-(methyloxy)ethyl]-N-[2-(4-morpholinyl)ethyl]-1H-benzimidazole-5-carboxamide: 3-amino-4-{[2-(methyloxy)ethyl]amino}-N-[2-(4-morpholinyl)ethyl]benzamide (325 mg, 1.008 mmol), 2-(4-chlorophenyl)-1H-imidazole-4-carbaldehyde (208 mg, 1.008 mmol) and sodium bisulfite (105 mg, 1.008 mmol) were combined in water/ethanol mixture and microwaved at 150° C. for 5 minutes. Reaction was concentrated and purified by Biotage (25 g silica column, 1-7% MeOH/DCM (plus NH₄OH), 20 min.; 7%, 10 min.). 314 mg (61%) light yellow foam was obtained as desired product. ¹H NMR (400 MHz, DMSO-d6) δ 13.26 (br. s., 1H), 8.38 (t, J=5.56 Hz, 1H), 7.98-8.18 (m, 4H), 7.71-7.79 (m, 1H), 7.53-7.69 (m, 3H), 5.05 (br. s., 2H), 3.84 (t, J=5.43 Hz, 2H), 3.59 (t, J=4.55 Hz, 4H), 3.42 (q, J=6.57 Hz, 2H), 3.20 (s, 3H), 2.49 (br. s., 2H), 2.44 (br. s., 4H); MS (m/z) 509.3 (M+H)⁺.

The following compounds were prepared with procedures analogous to that described in Example 1.

TABLE 1

| | | MS | |
|---|---|---|---|
| Structure | Example | (m/z) | Notes |
| 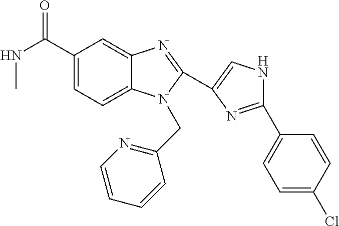 [SMILES: CNC(=O)c1ccc2c(c1)nc(-c1c[nH]c(-c3ccc(Cl)cc3)n1)n2Cc1ccccn1] | 2 | 443.1 | Step 4: Crude solid triturated in DCM, then crystallized from MeOH. |
| 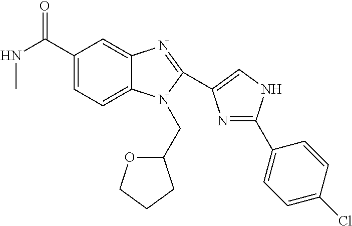 [SMILES: CNC(=O)c1ccc2c(c1)nc(-c1c[nH]c(-c3ccc(Cl)cc3)n1)n2CC1CCCO1] | 3 | 436.1 | Step 4: Solid was crystallized from 9:1 MeOH:water. |
| 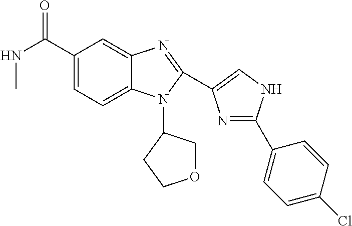 [SMILES: CNC(=O)c1ccc2c(c1)nc(-c1c[nH]c(-c3ccc(Cl)cc3)n1)n2C1CCOC1] | 4 | 422.1 | Step 4: Column purification, MeOH/DCM/NH₄OH gradient |
| 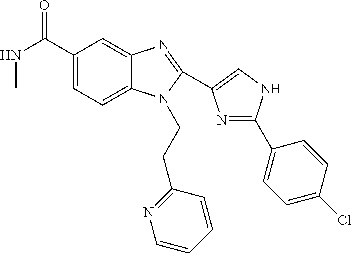 [SMILES: CNC(=O)c1ccc2c(c1)nc(-c1c[nH]c(-c3ccc(Cl)cc3)n1)n2CCc1ccccn1] | 5 | 457.1 | None. |

### Example 6

### 2-{2-[2-fluoro-5-(methyloxy)phenyl]-1H-imidazol-4-yl}-N-methyl-1-[2-(methyloxy)ethyl]-1H-benzimidazole-5-carboxamide

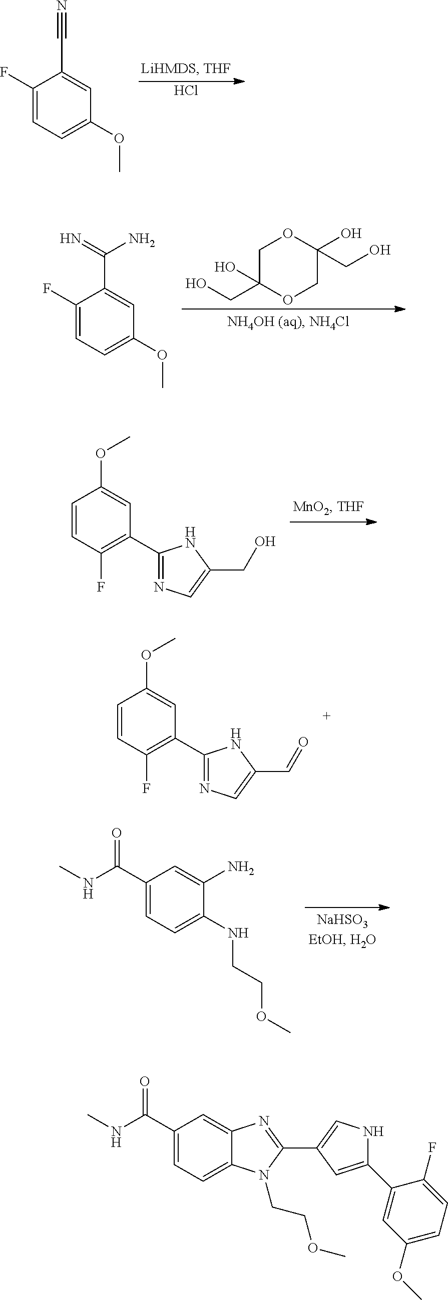

Step 1. 2-fluoro-5-(methyloxy)benzenecarboximidamide: To a dry reaction flask under nitrogen was added LiHMDS solution (13.2 mL, 13.2 mmol, 1M in THF). Next 2-fluoro-5-(methyloxy)benzonitrile (2.0 g, 13.2 mmol) was added slowly and the reaction was stirred at room temperature overnight. The reaction mixture was cooled in an ice/brine bath and HCl (15 mL, 4M in dioxane, mixed with 10 mL i-PrOH) was added dropwise. The mixture was warmed to room temperature and a solid formed. The solid was filtered, rinsed with diethyl ether and dried to give the title compound as an off-white solid (2.74 g, quant.). ¹H NMR (400 MHz, METHANOL-d₄) δ 7.25-7.35 (m, 2H), 7.22 (dd, J=2.78, 5.31 Hz, 1H), 3.88 (s, 3H); MS (m/z) 169.0 (M+H)⁺.

Step 2. {2-[2-fluoro-5-(methyloxy)phenyl]-1H-imidazol-4-yl}methanol: To 2,5-bis(hydroxymethyl)-1,4-dioxane-2,5-diol (2.64 g, 14.68 mmol) and 2-fluoro-5-(methyloxy)benzenecarboximidamide (2.73 g, 13.34 mmol) was added ammonium hydroxide (27 mL, 400 mmol, 28% in water) and ammonium chloride (3.77 g, 70.4 mmol). The reaction was stirred at 80° C. for 1.5 hours. DCM was added and the layers were separated. A solid precipitated out of the organic layer and it was filtered and dried to give the title compound as an orange/tan solid (1.50 g). The aqueous layer was extracted two times with 10% MeOH/DCM. Organics were combined and concentrated to give a solid which was triturated in DCM, filtered and dried to give a second batch of the title compound as an orange/tan solid (311 mg). 61% combined yield. ¹H NMR (400 MHz, DMSO-d₆) δ 12.05 (br. m., 1H), 7.47 (dd, J=3.28, 6.06 Hz, 1H), 7.25 (dd, J=9.09, 10.61 Hz, 1H), 7.08 (s, 1H), 6.93 (m, 1H), 5.05 (br. s., 0.35H), 4.91 (br. t., J=5.18 Hz, 0.65H), 4.37-4.54 (m, 2H), 3.79 (s, 3H); MS (m/z) 222.9 W.

Step 3. 2-[2-fluoro-5-(methyloxy)phenyl]-1H-imidazole-4-carbaldehyde: To a solution of {2-[2-fluoro-5-(methyloxy)phenyl]-1H-imidazol-4-yl}methanol (0.50 g, 2.25 mmol) in chloroform (10 mL) under nitrogen was added manganese dioxide (1.17 g, 13.5 mmol). The reaction was heated at 80° C. for 4 hours. Reaction mixture was filtered through hard packed Celite, rinsing with methanol, but not all MnO₂ was removed. The filtrate was run through a 0.45 μm nylon acrodisc to remove the remaining MnO₂. The filtrate was concentrated and purified by column chromatography, Biotage (25 g silica column; 0-5% Methanol/DCM, 15 min; 5%, 10 min) to give the title compound as a light yellow solid (368 mg, 70%). ¹H NMR (400 MHz, METHANOL-d₄) δ 9.84 (s, 1H), 8.04 (s, 1H), 7.62 (br. s., 1H), 7.22 (dd, J=9.22, 10.48 Hz, 1H), 7.05 (m, 1H), 3.87 (s, 3H); MS (m/z) 221.0 (M+H)⁺.

Step 4. 2-{2-[2-fluoro-5-(methyloxy)phenyl]-1H-imidazol-4-yl}-N-methyl-1-[2-(methyloxy)ethyl]-1H-benzimidazole-5-carboxamide: 3-Amino-N-methyl-4-{[2-(methyloxy)ethyl]amino}benzamide (0.150 g, 0.672 mmol), 2-[2-fluoro-5-(methyloxy)phenyl]-1H-imidazole-4-carbaldehyde (0.148 g, 0.672 mmol) and sodium bisulfite (0.140 g, 1.344 mmol) were combined in water (0.5 mL) and ethanol (2 mL) and microwaved at 150° C. for 5 minutes. The reaction was filtered while still warm and filtrate was concentrated and purified by Biotage (10 g silica column; (MeOH contains 1% NH₄OH); 0-6% MeOH/DCM, 15 min; 6%, 10 min.) to give the title compound as a light brown foam (207 mg, 71%). ¹H NMR (400 MHz, METHANOL-d₄) δ 8.12 (s, 1H), 7.99 (s, 1H), 7.76-7.82 (m, 1H), 7.63-7.71 (m, 2H), 7.16-7.24 (m, 1H), 6.95-7.03 (m, 1H), 5.04 (t, J=5.43 Hz, 2H), 3.94 (t, J=5.43 Hz, 2H), 3.88 (s, 3H), 3.30 (s, 3H), 2.98 (s, 3H); MS (m/z) 424.2 (M+H)⁺.

The following compounds were prepared with procedures analogous to that described in Example 6.

TABLE 2

| Structure | Example | MS (m/z) | ¹H NMR |
|---|---|---|---|
| 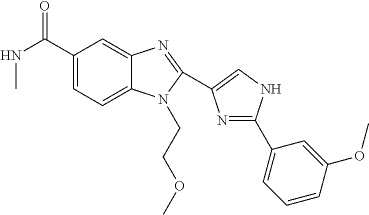 [SMILES: CNC(=O)c1ccc2c(c1)nc(-c1c[nH]c(-c3cccc(OC)c3)n1)n2CCOC] | 7 | 406.2 | ¹H NMR (400 MHz, ACETONITRILE-d₃) δ 11.14 (br. s., 1H), 8.03 (s, 1H), 7.95 (s, 1H), 7.69-7.76 (m, 1H), 7.54-7.62 (m, 3H), 7.44 (t, J = 7.96 Hz, 1H), 7.02 (dd, J = 2.15, 7.96 Hz, 2H), 5.07 (t, J = 5.56 Hz, 2H), 3.93 (t, J = 5.56 Hz, 2H), 3.90 (s, 3H), 3.31 (s, 3H), 2.93 (d, J = 4.80 Hz, 3H) |
| 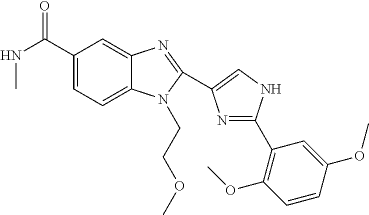 [SMILES: CNC(=O)c1ccc2c(c1)nc(-c1c[nH]c(-c3cc(OC)ccc3OC)n1)n2CCOC] | 8 | 436.2 | ¹H NMR (400 MHz, ACETONITRILE-d₃) δ 11.35 (br. s., 1H), 8.02 (s, 1H), 7.91 (br. s., 1H), 7.88 (d, J = 3.28 Hz, 1H), 7.72 (d, J = 8.59 Hz, 1H), 7.58 (d, J = 8.59 Hz, 1H), 7.12 (d, J = 8.84 Hz, 1H), 7.03 (br. s., 1H), 6.99 (dd, J = 3.28, 9.09 Hz, 1H), 5.00-5.12 (m, 2H), 4.01 (s, 3H), 3.95 (t, J = 5.56 Hz, 2H), 3.86 (s, 3H), 3.27 (s, 3H), 2.93 (d, J = 4.55 Hz, 3H) |
| 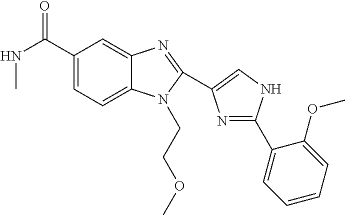 [SMILES: CNC(=O)c1ccc2c(c1)nc(-c1c[nH]c(-c3ccccc3OC)n1)n2CCOC] | 9 | 406.2 | |
| 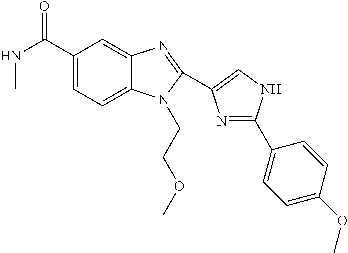 [SMILES: CNC(=O)c1ccc2c(c1)nc(-c1c[nH]c(-c3ccc(OC)cc3)n1)n2CCOC] | 10 | 406.2 | ¹H NMR (400 MHz, METHANOL-d₄) δ 8.13 (s, 1H), 7.87-7.97 (m, 3H), 7.80 (d, J = 8.84 Hz, 1H), 7.68 (d, J = 8.59 Hz, 1H), 7.06 (d, J = 8.84 Hz, 2H), 5.05 (br. s., 2H), 3.93 (t, J = 5.31 Hz, 2H), 3.88 (s, 3H), 3.29 (s, 3H), 2.98 (s, 3H) |

### Example 11

### 2-{2-[4-chloro-3-(methyloxy)phenyl]-1H-imidazol-4-yl}-N,N-dimethyl-1-[2-(methyloxy)ethyl]-1H-benzimidazole-5-carboxamide

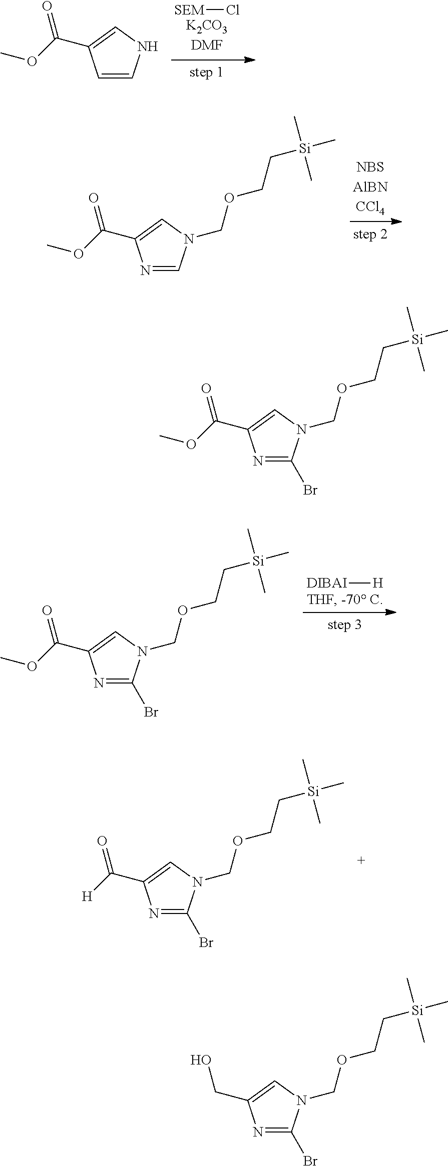

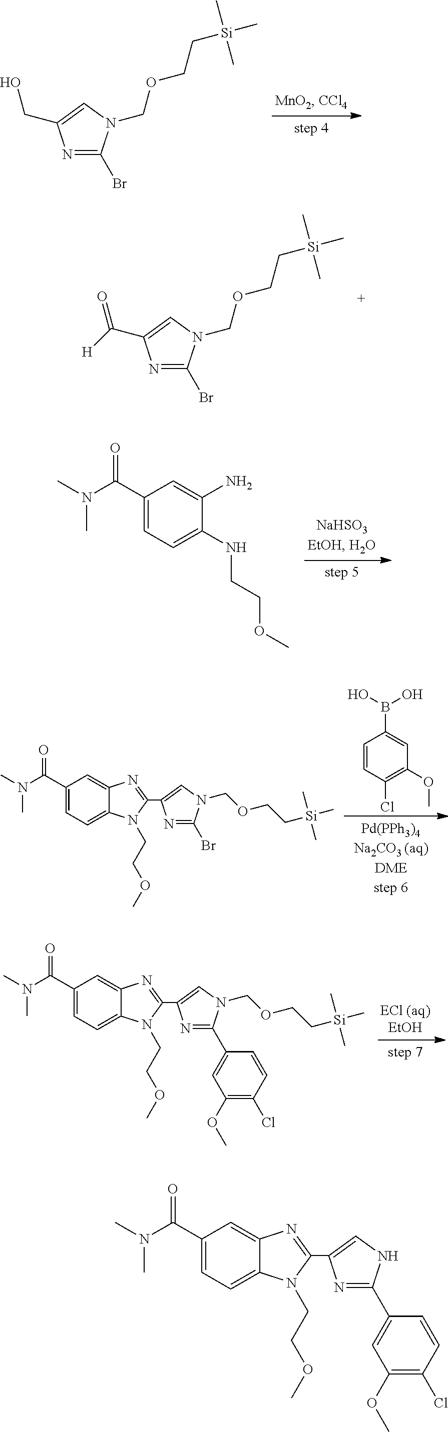

Step 1. methyl 1-({[2-(trimethylsilyl)ethyl]oxy}methyl)-1H-imidazole-4-carboxylate: A mixture of methyl 1H-imidazole-4-carboxylate (2.80 g, 22.20 mmol), {2-[(chloromethyl)oxy]ethyl}(trimethyl)silane (5.11 mL, 28.9 mmol) and potassium carbonate (7.67 g, 55.5 mmol) in DMF (20 mL) was heated at 80° C. for 24 hours. Reaction showed ˜1:1 starting material:desired product by TLC. The reaction was cooled to room temperature, diluted with ethyl acetate and water, and layers were separated. Organics were washed with brine, concentrated and purified by Biotage (50 g silica column, 10-75% E/H, 20 min.; 75%, 10 min.). Clean fractions were combined, concentrated and dried to give the title compound as a light yellow oil (2.46 g, 45%). ¹H NMR (400 MHz, DMSO-d₆) δ 8.02 (d, J=1.26 Hz, 1H), 7.92 (d, J=1.26 Hz, 1H), 5.37 (s, 2H), 3.74 (s, 3H), 3.44-3.52 (m, 2H), 0.80-0.88 (m, 2H), −0.03 (s, 9H); MS (m/z) 257.0 (M+H)⁺.

Step 2. methyl 2-bromo-1-({[2-(trimethylsilyl)ethyl]oxy}methyl)-1H-imidazole-4-carboxylate: Methyl 1-({[2-(trimethylsilyl)ethyl]oxy}methyl)-1H-imidazole-4-carboxylate (200 mg, 0.780 mmol) was dissolved in chloroform (4 mL) and NBS (146 mg, 0.819 mmol) and AIBN (6.41 mg, 0.039 mmol) were added. Reaction was heated at 60° C. for 3 hours. Reaction was diluted with satd. NaHCO₃ and layers were separated. Organics were concentrated and purified by Biotage (10 g silica column, 10-40% E/H, 15 min.) to give the title compound as colorless oil, which solidified upon standing (161 mg, 60%). ¹H NMR (400 MHz, DMSO-d₆) δ 8.26 (s, 1H), 5.34 (s, 2H), 3.76, (s, 3H), 3.54 (t, J=7.96 Hz, 2H), 0.85 (t, J=7.96 Hz, 2H), 0.03 (s, 9H); MS (m/z) 335.0/337.0, (M+H)⁺, shows Br pattern.

Step 3. 2-bromo-1-({[2-(trimethylsilyl)ethyl]oxy}methyl)-1H-imidazole-4-carbaldehyde and [2-bromo-1-({[2-(trimethylsilyl)ethyl]oxy}methyl)-1H-imidazol-4-yl]methanol: Methyl 2-bromo-1-({[2-(trimethylsilyl)ethyl]oxy}methyl)-1H-imidazole-4-carboxylate (1.71 g, 5.10 mmol) was dissolved in tetrahydrofuran (THF) (10 mL) under nitrogen and cooled in an acetone/dry ice bath. DIBAI-H (7.65 mL, 7.65 mmol, 1M in toluene) was added dropwise and the reaction was stirred cooled for 1 hour. There was no increase in product peak by LCMS from 30 to 60 minutes, so another 2 mL DIBAI-H solution was added and the reaction was warmed to room temperature and stirred overnight. LCMS showed a mixture of aldehyde and alcohol products. The reaction was cooled in an ice/water bath and water (2 mL) was added carefully to quench and the mixture was concentrated. DCM was added and the mixture was stirred for 30 minutes and aluminum salts precipitated out. The mixture was filtered through Celite, rinsing with DCM. The filtrate was concentrated and purified by Biotage (25 g silica column, 5-40% E/H, 15 min. to elute aldehyde and 80% E/H, 10 min to elute alcohol) to give title compounds (aldehyde, 376 mg white solid, 24% and alcohol, 1.04 g pale yellow oil, 66%).

Aldehyde: ¹H NMR (400 MHz, DMSO-d₆) δ 9.67 (s, 1H), 8.39 (s, 1H), 5.39 (s, 2H), 3.49-3.62 (t, J=8.08 Hz, 2H), 0.86 (t, J=8.08 Hz, 2H), −0.03 (m, 9H); MS (m/z) 304.9/306.9, (M+H)⁺, shows Br pattern.

Alcohol: ¹H NMR (400 MHz, DMSO-d₆) δ 7.29 (s, 1H), 5.24 (s, 2H), 5.00 (t, J=5.68 Hz, 1H), 4.29 (d, J=5.05 Hz, 2H), 3.47-3.55 (m, 2H), 0.81-0.89 (m, 2H), −0.03 (s, 9H) MS (m/z) 306.9/308.9, (M+H)⁺, shows Br pattern.

Step 4. 2-bromo-1-({[2-(trimethylsilyl)ethyl]oxy}methyl)-1H-imidazole-4-carbaldehyde: To a solution of [2-bromo-1-({[2-(trimethylsilyl)ethyl]oxy}methyl)-1H-imidazol-4-yl]methanol (320 mg, 1.04 mmol) dissolved in chloroform (10 mL) under nitrogen was added manganese dioxide (540 mg, 6.2 mmol, activated, <5 micron). The reaction was heated at 80° C. for 4 hours. The mixture was filtered through hard packed Celite, rinsing with DCM, but some manganese dioxide comes through. Filtrate was run through a 0.45 μm nylon acrodisc to remove remaining manganese dioxide. Filtrate was concentrated and dried to give the title compound as a white solid (278 mg, 88%). ¹H NMR (400 MHz, DMSO-d₆) δ 9.67 (s, 1H), 8.39 (s, 1H), 5.39 (s, 2H), 3.49-3.62 (t, J=8.08 Hz, 2H), 0.86 (t, J=8.08 Hz, 2H), −0.03 (m, 9H); MS (m/z) 304.9/306.9; MS (m/z) 304.9/306.9, (M+H)⁺, shows Br pattern.

Step 5. 2-[2-bromo-1-({[2-(trimethylsilyl)ethyl]oxy}methyl)-1H-imidazol-4-yl]-N,N-dimethyl-1-[2-(methyloxy)ethyl]-1H-benzimidazole-5-carboxamide: 3-amino-N,N-dimethyl-4-{[2-(methyloxy)ethyl]amino}benzamide (233 mg, 0.982 mmol), 2-bromo-1-({[2-(trimethylsilyl)ethyl]oxy}methyl)-1H-imidazole-4-carbaldehyde (300 mg, 0.982 mmol) and sodium bisulfite (102 mg, 0.982 mmol) were combined in water (0.50 mL) and ethanol (3 mL) and microwaved at 150° C. for 5 minutes. The reaction was concentrated and purified by Biotage (12 g silica column, 0.5-2.5% MeOH/DCM (plus NH₄OH), 20 min.) to give the title compound as a light yellow foam (307 mg, 57%). ¹H NMR (400 MHz, CHLOROFORM-d) δ 8.00 (br. s., 1H), 7.78 (s, 1H), 7.53 (d, J=8.34 Hz, 1H), 7.42 (dd, J=1.52, 8.34 Hz, 1H), 5.37 (s, 2H), 4.92 (t, J=5.68 Hz, 2H), 3.86 (t, J=5.56 Hz, 2H), 3.59-3.66 (m, 2H), 3.32 (s, 3H), 3.07-3.17 (br. d., 6H), 0.94-1.01 (m, 2H), 0.02 (s, 9H); MS (m/z) 522.1/524.1, (M+H)⁺, shows Br pattern.

Step 6. 2-[2-[4-chloro-3-(methyloxy)phenyl]-1-({[2-(trimethylsilyl)ethyl]oxy}methyl)-1H-imidazol-4-yl]-N,N-dimethyl-1-[2-(methyloxy)ethyl]-1H-benzimidazole-5-carboxamide: 2-[2-bromo-1-({[2-(trimethylsilyl)ethyl]oxy}methyl)-1H-imidazol-4-yl]-N,N-dimethyl-1-[2-(methyloxy)ethyl]-1H-benzimidazole-5-carboxamide (140 mg, 0.255 mmol),[4-chloro-3-(methyloxy)phenyl]boronic acid (56.9 mg, 0.305 mmol), tetrakis(triphenylphosphine) palladium(0) (29.4 mg, 0.025 mmol) and sodium carbonate (0.255 mL, 0.764 mmol) were combined in 1,2-dimethoxyethane in a microwave vial and heated in microwave at 160° C. for 30 min. The reaction was concentrated and purified by Biotage (12 g silica column; (MeOH contains 1% NH₄OH); 0.5-3% MeOH/DCM, 30 min; 3%, 10 min) to give the title compound as a light yellow solid (125 mg, 80%). ¹H NMR (400 MHz, DMSO-d₆) δ 8.36 (s, 1H), 7.58-7.70 (m, 4H), 7.50 (dd, J=1.89, 8.21 Hz, 1H), 7.29 (dd, J=1.64, 8.21 Hz, 1H), 5.55 (s, 2H), 5.01 (t, J=5.56 Hz, 2H), 3.95 (s, 3H), 3.83 (t, J=5.43 Hz, 2H), 3.65-3.72 (m, 2H), 3.20 (s, 3H), 3.01 (s, 6H), 0.87-0.97 (m, 2H), −0.03 (s, 9H); MS (m/z) 584.2, (M+H)⁺.

Step 7. 2-{2-[4-chloro-3-(methyloxy)phenyl]-1H-imidazol-4-yl}-N,N-dimethyl-1-[2-(methyloxy)ethyl]-1H-benzimidazole-5-carboxamide: 2-[2-[4-chloro-3-(methyloxy)phenyl]-1-({[2-(trimethylsilyl)ethyl]oxy}methyl)-1H-imidazol-4-yl]-N,N-dimethyl-1-[2-(methyloxy)ethyl]-1H-benzimidazole-5-carboxamide (40 mg, 0.065 mmol) was dissolved in ethanol (2 mL) and HCl (0.078 mL, 0.470 mmol, 6M in water) was added. The reaction was heated at 80° C. for 6 hours. Mixture was concentrated and purified by Biotage (12 g silica column, 0.5-6.5% MeOH/DCM (plus NH₄OH), 20 min.; 6.5%, 10 min.) to give the title compound as an off-white solid (20 mg, 66%). ¹H NMR (400 MHz, DMSO-d₆) δ13.29 (br. s., 1H), 8.15 (s, 1H), 7.81 (s, 1H), 7.53-7.71 (m, 4H), 7.28 (d, J=7.83 Hz, 1H), 5.04 (br. s., 2H), 3.97 (s, 3H), 3.87 (t, J=5.31 Hz, 2H), 3.23 (s, 3H), 3.01 (s, 6H); MS (m/z) 454.2, (M+H)⁺.

### Example 12

### 2-[2-(4-chloro-3-hydroxyphenyl)-1H-imidazol-4-yl]-N,N-dimethyl-1-[2-(methyloxy)ethyl]-1H-benzimidazole-5-carboxamide

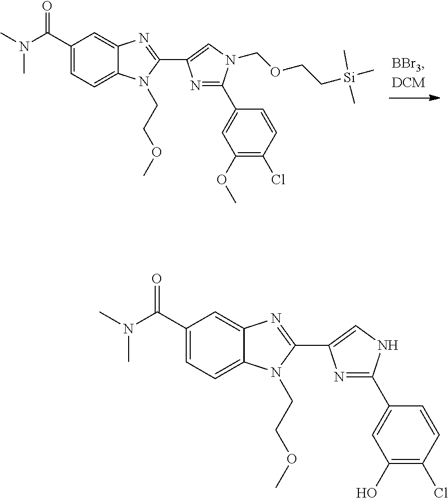

2-[2-[4-Chloro-3-(methyloxy)phenyl]-1-({[2-(trimethylsilyl)ethyl]oxy}methyl)-1H-imidazol-4-yl]-N,N-dimethyl-1-[2-(methyloxy)ethyl]-1H-benzimidazole-5-carboxamide (41 mg, 0.067 mmol) was dissolved in DCM (2 mL) under nitrogen and cooled in an ice/brine bath. Then boron tribromide (0.133 mL, 0.133 mmol, 1M in DCM) was added and reaction was allowed to warm to room temperature and was stirred for 4 hours. Reaction was not complete by LCMS. Reaction was cooled again in an ice/brine bath and boron tribromide (0.067 mL, 0.067 mmol, 1M in DCM) was added. Reaction was warmed to room temperature and stirred for 16 hours. Reaction was nearly complete. Cooled in ice/brine bath again and added more boron tribromide (0.133 mL, 0.133 mmol, 1M in DCM). Warmed to room temperature and stirred for another 24 hours. Reaction was complete. Reaction was quenched carefully with water (0.5 mL) and methanol (3 mL) and stirred for 10 minutes. Mixture was concentrated and purified by Biotage (12 g silica column, 0.5-8% MeOH/DCM (plus NH₄OH), 20 min.; 8%, 10 min.) to give the title compound as an off-white solid (23 mg, 76%). ¹H NMR (400 MHz, DMSO-d₆) δ 13.30 (br. s., 1H), 8.14 (s, 1H), 7.81 (s, 1H), 7.60-7.72 (m, 3H), 7.53-7.60 (m, 1H), 7.28 (d, J=8.08 Hz, 1H), 5.09 (br. s., 1H), 4.87 (br. s., 2H), 3.81-4.04 (m, 5H), 3.01 (s, 6H); MS (m/z) 440.3, (M+H)⁺.

The following compounds 13-28 were prepared with procedures analogous to that described in Example 11. Compound 29 was prepared with procedures analogous to that described in Example 12.

TABLE 3

| Structure | Example | MS (m/z) | ¹H NMR |
|---|---|---|---|
| 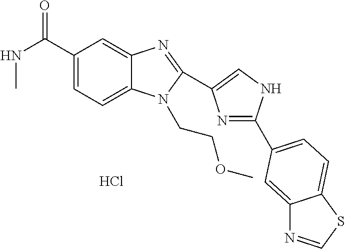 [SMILES: CNC(=O)c1ccc2c(c1)nc(-c1c[nH]c(-c3ccc4scnc4c3)n1)n2CCOC ; Cl] | 13 | 433.3 | ¹H NMR (400 MHz, DMSO-d₆) δ 14.18 (br. s., 1H), 9.53 (s, 1H), 8.85 (s, 1H), 8.70-8.81 (m, 2H), 8.39 (d, J = 8.34 Hz, 1H), 8.27 (d, J = 8.34 Hz, 1H), 8.23 (s, 1H), 8.05 (s, 2H), 5.18 (t, J = 4.55 Hz, 2H), 3.96 (t, J = 4.80 Hz, 2H), 3.23 (s, 3H), 2.85 (d, J = 4.29 Hz, 3H) |
| 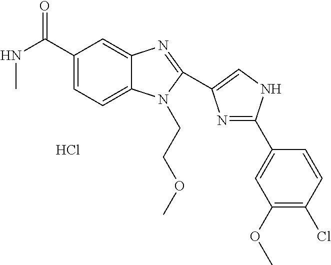 [SMILES: CNC(=O)c1ccc2c(c1)nc(-c1c[nH]c(-c3ccc(Cl)c(OC)c3)n1)n2CCOC ; Cl] | 14 | 440.2 | ¹H NMR (400 MHz, DMSO-d₆) δ 13.94 (br. s., 1H), 8.66 (br. s., 1H), 8.53 (br. s., 1H), 8.19 (s, 1H), 7.97 (br. s., 2H), 7.88 (br. s., 1H), 7.71 (dd, J = 1.64, 8.21 Hz, 1H), 7.63 (d, J = 8.34 Hz, 1H), 5.08 (br. s., 2H), 3.99 (s, 3H), 3.91 (t, J = 4.93 Hz, 2H), 3.22 (s, 3H), 2.85 (d, J = 4.29 Hz, 3H) |
| 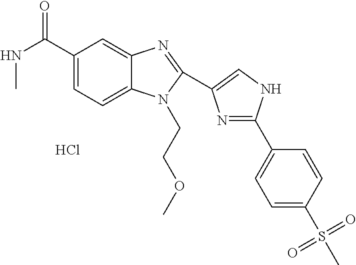 [SMILES: CNC(=O)c1ccc2c(c1)nc(-c1c[nH]c(-c3ccc(S(C)(=O)=O)cc3)n1)n2CCOC ; Cl] | 15 | 454.2 | |
| 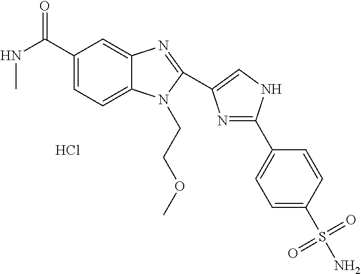 [SMILES: CNC(=O)c1ccc2c(c1)nc(-c1c[nH]c(-c3ccc(S(N)(=O)=O)cc3)n1)n2CCOC ; Cl] | 16 | 455.2 | |
| 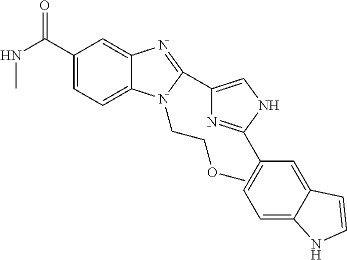 [SMILES: CNC(=O)c1ccc2c(c1)nc(-c1c[nH]c(-c3ccc4[nH]ccc4c3)n1)n2CCOC] | 17 | 415.3 | ¹H NMR (400 MHz, DMSO-d₆) δ 13.07 (br. s., 1H), 11.31 (br. s., 1H), 8.44 (br. s., 1H), 8.26 (s, 1H), 8.10 (s, 1H), 8.06 (br. s., 1H), 7.84 (dd, J = 1.64, 8.46 Hz, 1H), 7.78 br. m., 1H), 7.71 (br. m., 1H), 7.52 (d, J = 8.59 Hz, 1H), 7.41-7.46 (m, 1H), 6.55 (br. s., 1H), 5.08 (br. s., 2H), 3.88 (t, J = 5.43 Hz, 2H), 3.23 (s, 3H), 2.83 (d, J = 4.55 Hz, 3H) |
| 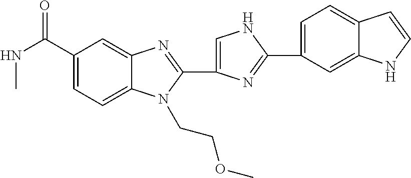 [SMILES: CNC(=O)c1ccc2c(c1)nc(-c1c[nH]c(-c3ccc4cc[nH]c4c3)n1)n2CCOC] | 18 | 415.3 | ¹H NMR (400 MHz, DMSO-d₆) δ 13.05 (br. s., 1H), 11.37 (s, 1H), 8.40 (d, J = 4.55 Hz, 1H), 8.06-8.11 (m, 2H), 8.02 (d, J = 2.02 Hz, 1H), 7.74 (ddd, J = 1.39, 4.67, 8.34 Hz, 2H), 7.64 (t, J = 7.71 Hz, 2H), 7.45 (t, J = 2.65 Hz, 1H), 6.48 (br. s., 1H), 5.10 (t, J = 5.43 Hz, 2H), 3.88 (t, J = 5.56 Hz, 2H), 3.23 (s, 3H), 2.82 (d, J = 4.55 Hz, 3H) |
| 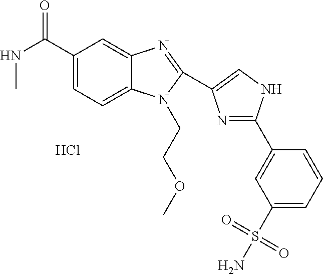 [SMILES: CNC(=O)c1ccc2c(c1)nc(-c1c[nH]c(-c3cccc(S(N)(=O)=O)c3)n1)n2CCOC ; Cl] | 19 | 455.3 | |
| 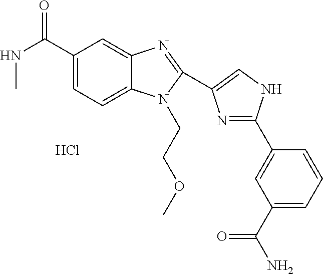 [SMILES: CNC(=O)c1ccc2c(c1)nc(-c1c[nH]c(-c3cccc(C(N)=O)c3)n1)n2CCOC ; Cl] | 20 | 419.3 | |
| 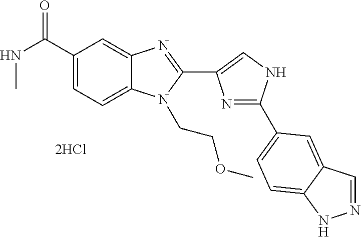 [SMILES: CNC(=O)c1ccc2c(c1)nc(-c1c[nH]c(-c3ccc4[nH]ncc4c3)n1)n2CCOC ; "2HCl"] | 21 | 416.2 | ¹H NMR (400 MHz, DMSO-d₆) δ 14.04 (br. s., 1H), 13.34 (br. s., 1H), 8.77 (d, J = 4.29 Hz, 1H), 8.67 (s, 1H), 8.55 (s, 1H), 8.27 (s, 1H), 8.23 (s, 1H), 8.11-8.18 (m, 1H), 8.04 (s, 2H), 7.74 (d, J = 8.84 Hz, 1H), 5.10- 5.21 (m, 2H), 3.94 (t, J = 4.80 Hz, 2H), 3.22 (s, 3H), 2.85 (d, J = 4.29 Hz, 3H) |
| 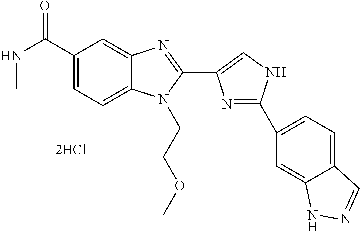 [SMILES: CNC(=O)c1ccc2c(c1)nc(-c1c[nH]c(-c3ccc4cn[nH]c4c3)n1)n2CCOC ; "2HCl"] | 22 | 416.3 | ¹H NMR (400 MHz, DMSO-d₆) δ 14.08 (br. s., 1H), 13.47 (br. s., 1H), 8.74 (br. s., 1H), 8.65 (s, 1H), 8.31 (s, 1H), 8.22 (s, 1H), 8.18 (s, 1H), 8.03 (s, 2H), 7.86-7.98 (m, 2H), 5.15 (br. s., 2H), 3.94 (br. m., 2H), 3.23 (s, 3H), 2.85 (d. J = 4.04 Hz, 3H) |
| 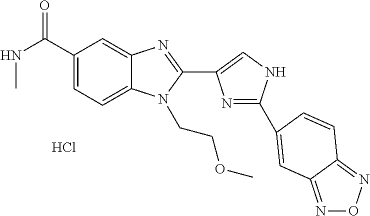 [SMILES: CNC(=O)c1ccc2c(c1)nc(-c1c[nH]c(-c3ccc4nonc4c3)n1)n2CCOC ; Cl] | 23 | 418.2 | ¹H NMR (400 MHz, DMSO-d₆) δ 14.19 (br. s., 1H), 8.68 (s, 1H), 8.63 (br. s., 1H), 8.59 (br. s., 1H), 8.41 (d, J = 9.35 Hz, 1H), 8.28 (d, J = 9.35 Hz, 1H), 8.19 (s, 1H), 7.94 (br. s., 2H), 5.12 (br. s., 2H), 3.92 (t, J = 4.93 Hz, 2H), 3.22 (s, 3H), 2.85 (d, J = 4.29 Hz, 3H) |
| 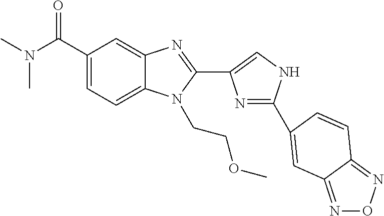 [SMILES: COCCn1c(-c2c[nH]c(-c3ccc4nonc4c3)n2)nc2cc(C(=O)N(C)C)ccc21] | 24 | 432.2 | ¹H NMR (400 MHz, DMSO-d₆) δ 13.71 (br. s., 1H), 8.56 (s, 1H), 8.36- 8.41 (m, 1H), 8.32 (d, J = 2.27 Hz, 1H), 8.24 (d, J = 9.35 Hz, 1H), 7.67 (d, J = 8.34 Hz, 1H), 7.61-7.64 (m, 1H), 7.30 (dd, J = 1.26, 8.34 Hz, 1H), 5.07 (t, J = 5.43 Hz, 2H), 3.89 (t, J = 5.31 Hz, 2H), 3.23 (s, 3H), 3.01 (s, 6H) |
| 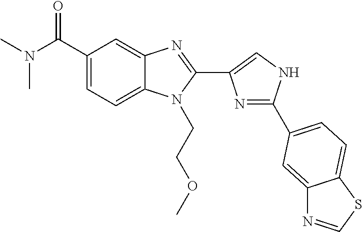 [SMILES: COCCn1c(-c2c[nH]c(-c3ccc4scnc4c3)n2)nc2cc(C(=O)N(C)C)ccc21] | 25 | 447.1 | ¹H NMR (400 MHz, DMSO-d₆) δ 13.35 (br. s., 1H), 9.49 (s, 1H), 8.75 (d, J = 1.26 Hz, 1H), 8.32 (d, J = 8.59 Hz, 1H), 8.18-8.23 (m, 1H), 8.14 (s, 1H), 7.59-7.68 (m, 2H), 7.28 (dd, J = 1.39, 8.21 Hz, 1H), 5.10 (t, J = 5.43 Hz, 2H), 3.91 (t, J = 5.43 Hz, 2H), 3.24 (s, 3H), 3.01 (s, 6H) |
| 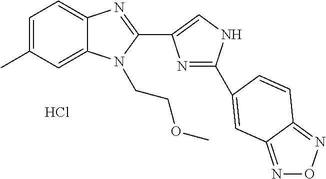 [SMILES: COCCn1c(-c2c[nH]c(-c3ccc4nonc4c3)n2)nc2ccc(C)cc21 ; Cl] | 26 | 375.1 | ¹H NMR (400 MHz, DMSO-d₆) δ 14.33 (br. s., 1H), 8.72 (s, 1H), 8.64 (br. s., 1H), 8.41 (d, J = 9.35 Hz, 1H), 8.29 (d, J = 9.35 Hz, 1H), 7.80 (br. s.,1H), 7.69 (d, J = 8.34 Hz, 1H), 7.40 (br. s., 1H), 5.10 (br. s., 2H), 3.92 (br. m., 2H), 3.22 (s, 3H), 2.53 (s, 3H) |
| 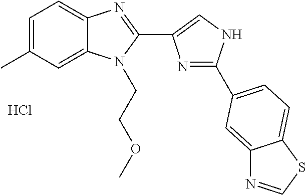 [SMILES: COCCn1c(-c2c[nH]c(-c3ccc4scnc4c3)n2)nc2ccc(C)cc21 ; Cl] | 27 | 390.1 | ¹H NMR (400 MHz, DMSO-d₆) δ 14.04 (br. s., 1H), 9.53 (s, 1H), 8.83 (s, 1H), 8.60 (s, 1H), 8.38 (d, J = 8.34 Hz, 1H), 8.26 (d, J = 8.34 Hz, 1H), 7.82 (s, 1H), 7.69 (d, J = 8.34 Hz, 1H), 7.41 (d, J = 8.08 Hz, 1H), 5.05-5.18 (br. m., 2H), 3.94 (t, J = 4.67 Hz, 2H), 3.23 (s, 3H), 2.53 (s, 3H) |
| 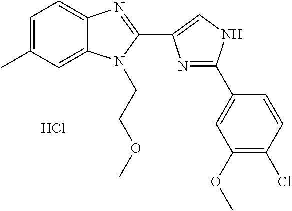 [SMILES: COCCn1c(-c2c[nH]c(-c3ccc(Cl)c(OC)c3)n2)nc2ccc(C)cc21 ; Cl] | 28 | 397.1 | ¹H NMR (400 MHz, DMSO-d₆) δ 14.09 (br. s., 1H), 8.56 (br. s., 1H), 7.90 (br. s., 1H), 7.80 (s, 1H), 7.59- 7.76 (m, 3H), 7.40 (d, J = 8.08 Hz, 1H), 5.06 (br. s., 2H), 4.00 (s, 3H), 3.91 (t, J = 4.80 Hz, 2H), 3.22 (s, 3H), 2.53 (s, 2H) |
| 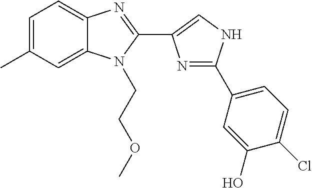 [SMILES: COCCn1c(-c2c[nH]c(-c3ccc(Cl)c(O)c3)n2)nc2ccc(C)cc21] | 29 | 383.2 | ¹H NMR (400 MHz, DMSO-d₆) δ 13.22 (br. s., 1H), 8.02 (br. s., 1H), 7.82 (br. s., 1H), 7.64 (br. s., 1H), 7.55 (d, J = 8.08 Hz, 1H), 7.47, br. s., 1H), 7.40 (br. s., 1H), 7.03 (d, J = 8.08 Hz, 1H), 5.15 (br. s., 1H), 4.78 (br. s., 2H), 3.97 (s, 3H), 3.90 (br. s., 2H), 2.46 (s, 3H) |

### Example 30

### 2-[2-(4-chlorophenyl)-1H-imidazol-4-yl]-1-[2-(methyloxy)ethyl]-1H-benzimidazole

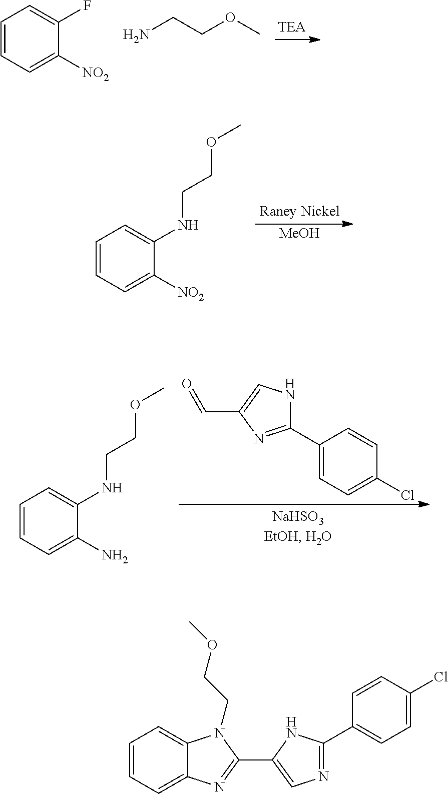

Step 1. [2-(methyloxy)ethyl](2-nitrophenyl)amine: 1-fluoro-2-nitrobenzene (100 mg, 0.709 mmol) was dissolved in Ethanol (400 μl) and DIEA (371 μl, 2.126 mmol) was added and added to a microwave vial. 2-(methyloxy)ethanamine (53.2 mg, 0.709 mmol) was added and the vial was capped and heated at 150° C. for 19 minutes. LCMS confirms complete conversion to the product. The solvent was removed and the product was carried on without further purification. Quantitative yield was assumed

Step 2. (2-aminophenyl)[2-(methyloxy)ethyl]amine: [2-(methyloxy)ethyl](2-nitrophenyl)amine (139 mg, 0.708 mmol) was dissolve up in Methanol (20 ml) and a catalytic amount of Raney Nickel was added. The reaction was allowed to stir under a hydrogen atmosphere until complete by LC/MS. The reaction was complete after 2 hours. The reaction was then filtered and the solvent was removed under vacuum. Quantitative yield was assumed and sample was carried on to the next reaction without further purification.

Step 3. 2-[2-(4-chlorophenyl)-1H-imidazol-4-yl]-1-[2-(methyloxy)ethyl]-1H-benzimidazole: (2-aminophenyl)[2-(methyloxy)ethyl]amine (118 mg, 0.710 mmol) was dissolved in Ethanol (1.5 mL) and then 2-(4-chlorophenyl)-1H-imidazole-4-carbaldehyde (147 mg, 0.710 mmol) was added along with sodium bisulfite (73.9 mg, 0.710 mmol) and Water (0.5 mL). The reaction was setup in a microwave vial, capped and heated in a Biotage microwave for 10 minutes at 150° C. The resulting compound was extracted with Ethyl Acetate and washed with Brine, water and then brine again. The ethyl acetate layer was dried over sodium sulphate and then later filtered. The solvent was removed and the crude product was purified by prep HPLC to afford pure product. 2-[2-(4-chlorophenyl)-1H-imidazol-4-yl]-1-[2-(methyloxy)ethyl]-1H-benzimidazole. ¹H NMR (400 MHz, CHLOROFORM-d) δ ppm 3.31 (s, 3H) 3.87 (t, J=5.05 Hz, 2H) 4.97 (t, J=5.18 Hz, 2H) 7.02 (d, J=8.59 Hz, 2H) 7.44-7.58 (m, 3H) 7.61-7.66 (m, 1H) 7.73 (d, J=8.34 Hz, 2H) 8.34 (s, 1H); MS (m/z) 353.1 (M+H)⁺.

The following compounds 30, 32-48 were prepared with procedures analogous to that described in Example 33.

| Structure | Example | m/z (M + H) | ¹H NMR |
|---|---|---|---|
| 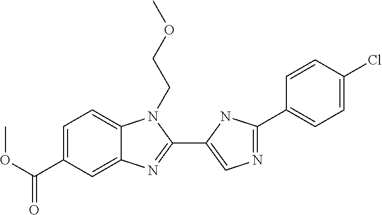 [SMILES: COCCn1c(-c2cnc(-c3ccc(Cl)cc3)[nH]2)nc2cc(C(=O)OC)ccc21] | 31 | 411.1 | ¹H NMR(400 MHz, CHLOROFORM-d) δ ppm 3.31 (s, 3 H) 3.91 (t, J = 4.80 Hz, 2 H) 4.03 (s, 3 H) 5.02 (t, J = 4.80 Hz, 2 H) 7.06 (d, J = 8.59 Hz, 2 H) 7.62 (d, J = 8.84 Hz, 1 H) 7.74 (d, J = 8.34 Hz, 2 H) 8.18 (dd, J = 8.59, 1.26 Hz, 1 H) 8.25 (d, J = 1.52 Hz, 1 H) 8.39 (s, 1 H) |
| 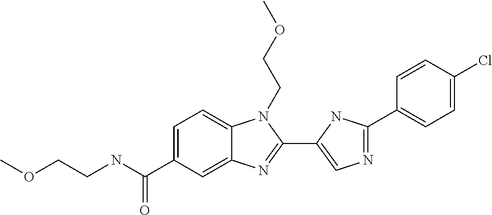 [SMILES: COCCNC(=O)c1ccc2c(c1)nc(-c1cnc(-c3ccc(Cl)cc3)[nH]1)n2CCOC] | 32 | 454.1 | ¹H NMR (400 MHz, DMSO-d₆) δ ppm 3.20 (s, 3 H) 3.29 (s, 3 H) 3.49 (s, 4 H) 3.87 (s, 2 H) 5.03- 5.10 (m, 2 H) 7.64 (d, J = 8.59 Hz, 2 H) 7.82-7.95 (m, 2 H) 8.09 (d, J = 8.34 Hz, 2 H) 8.15-8.18 (m, 1 H) 8.30-8.36 (m, 1 H) 8.67 (s, 1 H) |
| 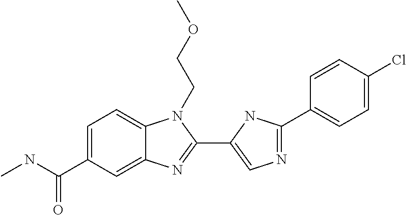 [SMILES: CNC(=O)c1ccc2c(c1)nc(-c1cnc(-c3ccc(Cl)cc3)[nH]1)n2CCOC] | 33 | 410.1 | ¹H NMR (400 MHz, DMSO-d₆) δ ppm 2.82 (d, J = 4.29 Hz, 3 H) 3.20 (s, 3 H) 3.84 (t, J = 5.31 Hz, 2 H) 4.99-5.08 (m, 2 H) 7.57- 7.66 (m, 4 H) 7.72-7.77 (m, 1 H) 8.04-8.14 (m, 4 H) 8.39-8.46 (m, 1 H) |
| 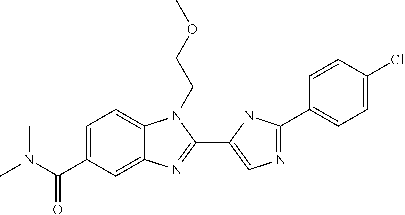 [SMILES: COCCn1c(-c2cnc(-c3ccc(Cl)cc3)[nH]2)nc2cc(C(=O)N(C)C)ccc21] | 34 | 424.1 | ¹H NMR (400 MHz, DMSO-d₆) δ ppm 2.92-3.08 (m, 6 H) 3.21 (s, 3 H) 3.90 (t, J = 5.18 Hz, 2 H) 5.05- 5.12 (m, 2 H) 7.48-7.53 (m, 1 H) 7.65 (d, J = 8.84 Hz, 2 H) 7.75 (s, 1 H) 7.91 (d, J = 8.34 Hz, 1 H) 8.10 (d, J = 8.59 Hz, 2 H) 8.41 (s, 1 H) |
| 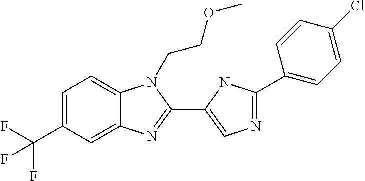 [SMILES: COCCn1c(-c2cnc(-c3ccc(Cl)cc3)[nH]2)nc2cc(C(F)(F)F)ccc21] | 35 | 421.1 | ¹H NMR (400 MHz, DMSO-d₆) δ ppm 3.20 (s, 3 H) 3.87 (t, J = 5.18 Hz, 2 H) 5.09 (t, J = 5.05 Hz, 2 H) 7.59-7.68 (m, 3 H) 7.89-7.99 (m, 2 H) 8.05-8.12 (m, 2 H) 8.27 (s, 1 H) |
| 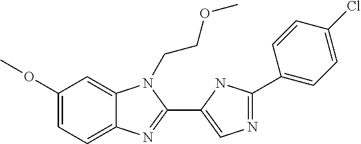 [SMILES: COCCn1c(-c2cnc(-c3ccc(Cl)cc3)[nH]2)nc2ccc(OC)cc21] | 36 | 383.1 | ¹H NMR (400 MHz, DMSO-d₆) δ ppm 3.22 (s, 3 H) 3.84-3.94 (m, 5 H) 5.07 (t, J = 5.05 Hz, 2 H) 7.15 (dd, J = 8.84, 2.27 Hz, 1 H) 7.51 (d, J = 2.02 Hz, 1 H) 7.63-7.71 (m, 3 H) 8.04-8.14 (m, 2 H) 8.40 (s, 1 H) |
| 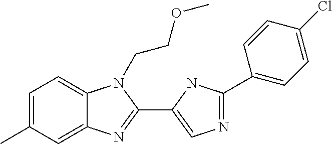 [SMILES: COCCn1c(-c2cnc(-c3ccc(Cl)cc3)[nH]2)nc2cc(C)ccc21] | 37 | 367.1 | ¹H NMR (400 MHz, DMSO-d₆) δ ppm 2.51 (s, 3 H) 3.20 (s, 3 H) 3.89 (t, J = 4.67 Hz, 2 H) 5.06 (t, J = 4.67 Hz, 2 H) 7.37 (d, J = 8.59 Hz, 1 H) 7.56 (s, 1 H) 7.66 (d, J = 8.59 Hz, 2 H) 7.83 (d, J = 8.59 Hz, 1 H) 8.10 (d, J = 8.59 Hz, 2 H) 8.45 (s, 1 H) |
| 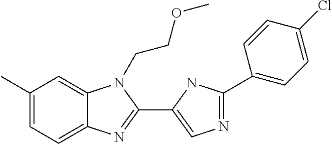 [SMILES: COCCn1c(-c2cnc(-c3ccc(Cl)cc3)[nH]2)nc2ccc(C)cc21] | 38 | 367.1 | ¹H NMR (400 MHz, DMSO-d₆) δ ppm 2.52 (s, 4 H) 3.21 (s, 3 H) 3.85-3.96 (m, 2 H) 5.00-5.14 (m, 2 H) 7.37 (d, J = 8.34 Hz, 1 H) 7.60-7.71 (m, 3 H) 7.76 (s, 1 H) 8.10 (d, J = 8.59 Hz, 2 H) 8.45 (s, 1 H) |
| 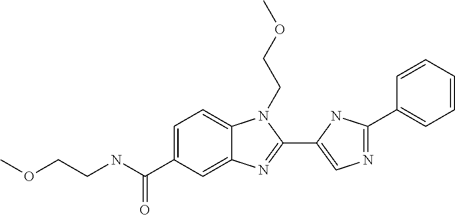 [SMILES: COCCNC(=O)c1ccc2c(c1)nc(-c1cnc(-c3ccccc3)[nH]1)n2CCOC] | 39 | 420.2 | ¹H NMR (400 MHz, DMSO-d₆) δ ppm 3.21 (s, 3 H) 3.29 (s, 3 H) 3.45-3.52 (m, 4 H) 3.90 (t, J = 5.05 Hz, 2 H) 5.09 (t, J = 4.80 Hz, 2 H) 7.49 (s, 1 H) 7.54-7.60 (m, 2 H) 7.88-7.98 (m, 2 H) 8.07- 8.11 (m, 2 H) 8.18 (d, J = 1.01 Hz, 1 H) 8.37 (s, 1 H) 8.71 (s, 1 H) |
| 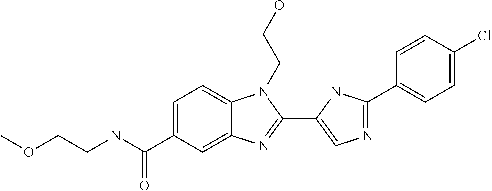 [SMILES: COCCNC(=O)c1ccc2c(c1)nc(-c1cnc(-c3ccc(Cl)cc3)[nH]1)n2CCO] | 40 | 440.1 | ¹H NMR (400 MHz, DMSO-d₆) δ ppm 3.29 (s, 3 H) 3.42-3.54 (m, 4 H) 3.95 (s, 2 H) 4.87-4.94 (m, 2 H) 7.65 (d, J = 8.59 Hz, 2 H) 7.87-8.00 (m, 2 H) 8.10 (d, J = 8.59 Hz, 2 H) 8.18 (s, 1 H) 8.36- 8.43 (m, 1 H) 8.67-8.77 (m, 1 H) |
| 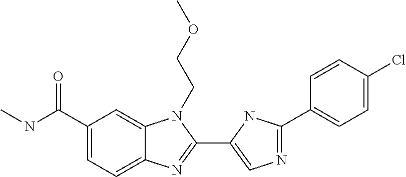 [SMILES: CNC(=O)c1ccc2nc(-c3cnc(-c4ccc(Cl)cc4)[nH]3)n(CCOC)c2c1] | 41 | 410.1 | ¹H NMR (400 MHz, DMSO-d₆) δ ppm 2.81-2.87 (m, 3 H) 3.21 (s, 3 H) 3.89 (t, 2 H) 5.06-5.11 (m, 2 H) 7.62-7.66 (m, 2 H) 7.69- 7.73 (m, 1 H) 7.84-7.89 (m, 1 H) 8.06-8.11 (m, 2 H) 8.25-8.32 (m, 2 H) 8.51-8.56 (m, 1 H) |
| 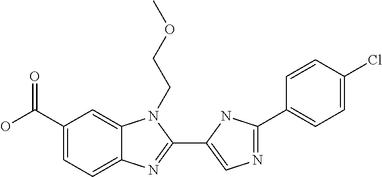 [SMILES: COCCn1c(-c2cnc(-c3ccc(Cl)cc3)[nH]2)nc2ccc(C(=O)O)cc21] | 42 | 397.1 | ¹H NMR (400 MHz, DMSO-d₆) δ ppm 3.20 (s, 3 H) 3.88 (t, J = 5.18 Hz, 2 H) 5.12 (t, J = 5.05 Hz, 2 H) 7.64 (d, J = 8.84 Hz, 2 H) 7.74 (d, J = 8.59 Hz, 1 H) 7.94-7.99 (m, 1 H) 8.06-8.12 (m, 2 H) 8.34 (d, J = 7.83 Hz, 2 H) |
| 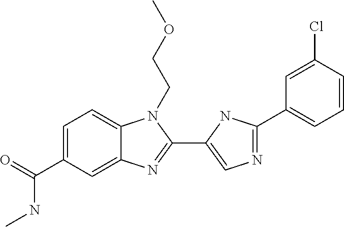 [SMILES: CNC(=O)c1ccc2c(c1)nc(-c1cnc(-c3cccc(Cl)c3)[nH]1)n2CCOC] | 43 | 410.1 | ¹H NMR (400 MHz, DMSO-d₆) δ ppm 2.84 (d, J = 4.29 Hz, 3 H) 3.21 (s, 3 H) 3.89 (t, J = 5.18 Hz, 2 H) 5.07 (t, J = 5.05 Hz, 2 H) 7.52- 7.63 (m, 2 H) 7.90 (d, J = 5.05 Hz, 2 H) 8.05 (d, J = 7.58 Hz, 1 H) 8.13-8.17 (m, 2 H) 8.38 (s, 1 H) 8.60 (m, 1 H) |
| 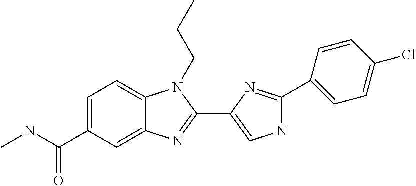 [SMILES: CCCn1c(-c2c[nH]c(-c3ccc(Cl)cc3)n2)nc2cc(C(=O)NC)ccc21] | 44 | 394 | ¹H NMR (400 MHz, DMSO-d₆) δ ppm 0.98 (t, J = 7.45 Hz, 3 H) 1.93 (d, J = 7.58 Hz, 2 H) 2.84 (d, J = 4.55 Hz, 3 H) 4.86 (d, J = 7.58 Hz, 2 H) 7.65 (d, J = 8.59 Hz, 2 H) 7.94 (br. s., 2 H) 8.10 (d, J = 8.59 Hz, 2 H) 8.16 (s, 1 H) 8.36 (br. s., 1 H) 8.51-8.67 (m, 1 H) |
| 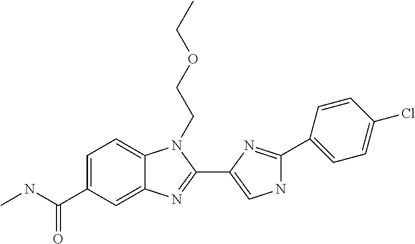 [SMILES: CCOCCn1c(-c2c[nH]c(-c3ccc(Cl)cc3)n2)nc2cc(C(=O)NC)ccc21] | 45 | 424 | ¹H NMR (400 MHz, DMSO-d₆) δ ppm 0.93 (t, J = 7.07 Hz, 3 H) 2.83 (d, J = 4.55 Hz, 3 H) 3.40 (q, J = 7.07 Hz, 2 H) 3.82-3.95 (m, 2 H) 4.93-5.15 (m, 2 H) 7.64 (d, J = 8.59 Hz, 2 H) 7.76-7.97 (m, 2 H) 8.09 (d, J = 8.59 Hz, 2 H) 8.12- 8.19 (m, 1 H) 8.25-8.38 (m, 1 H) 8.49-8.68 (m, 1 H) |
| 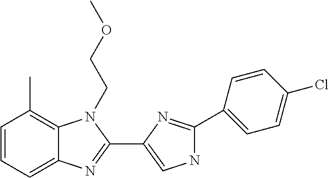 [SMILES: COCCn1c(-c2c[nH]c(-c3ccc(Cl)cc3)n2)nc2cccc(C)c21] | 46 | 367 | ¹H NMR (400 MHz, DMSO-d₆) δ ppm 2.81 (s, 3 H) 3.19 (s, 3H) 3.88 (t, J = 5.31 Hz, 4 H) 5.22 (br. s., 4 H) 7.27 (m, 1 H) 7.38 (m, 1 H) 7.60 (d, J = 8.08 Hz, 1 H) 7.66 (d, J = 8.34 Hz, 2 H) 8.09 (d, J = 8.59 Hz, 2 H) 8.40 (s, 1 H) |

### Example 47

### 2-[2-(4-chlorophenyl)-1H-imidazol-4-yl]-1-[2-(methyloxy)ethyl]-N-4-pyridinyl-1H-benzimidazole-5-carboxamide

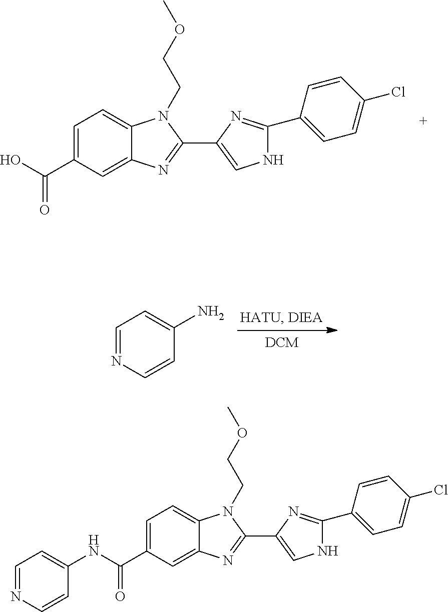 [SMILES: COCCn1c(-c2c[nH]c(-c3ccc(Cl)cc3)n2)nc2cc(C(=O)O)ccc21 ; Nc1ccncc1 ; COCCn1c(-c2c[nH]c(-c3ccc(Cl)cc3)n2)nc2cc(C(=O)Nc3ccncc3)ccc21]

2-[2-(4-Chlorophenyl)-1H-imidazol-4-yl]-1-[2-(methyloxy)ethyl]-1H-benzimidazole-5-carboxylic acid (8.0 mg, 0.020 mmol) was taken in DCM (0.5 mL) and 4-pyridinamine (1.89 mg, 0.020 mmol) was added along with HATU (8.43 mg, 0.022 mmol) and DIEA (5.28 uL, 0.030 mmol). After 20 min LC/MS shows the desired product as evidence by (M+H)=473. The solvent was evaporated in vacuo and the compound was diluted with DMSO and purified by prep HPLC. Analytical data was consistent with the structure. ¹H NMR (400 MHz, METHANOL-d₄) δ ppm 3.31 (s, 3H) 3.98-4.06 (m, 2H) 5.10-5.20 (m, 2 H) 7.52-7.60 (m, 2H) 7.98-8.09 (m, 3H) 8.17 (s, 1H) 8.35 (br. s., 1H) 8.39-8.48 (m, 3 H) 8.63-8.74 (m, 2H); MS (m/z) 473.0 (M+H)⁺.

The following compounds 49-52 were prepared with procedures analogous to that described in Example 53.

| Structure | Example | m/z (M + H) | ¹H NMR |
|---|---|---|---|
| 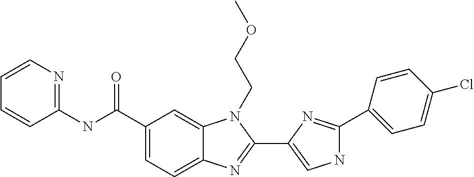 [SMILES: COCCn1c(-c2c[nH]c(-c3ccc(Cl)cc3)n2)nc2ccc(C(=O)Nc3ccccn3)cc21] | 48 | 473 | ¹H NMR (400 MHz, METHANOL-d₄) δ ppm 3.31 (s, 3H) 4.02-4.13 (m, 2 H) 5.12-5.25 (m, 2 H) 7.28-7.38 (m, 1 H) 7.52-7.64 (m, 2 H) 7.85- 7.95 (m, 1 H) 7.98-8.12 (m, 3 H) 8.14-8.29 (m, 2 H) 8.35-8.48 (m, 2 H) 8.54-8.65 (m, 1 H) |
| 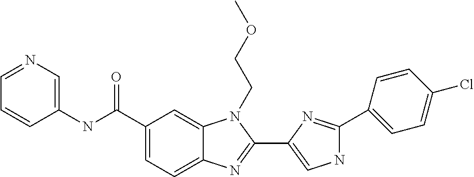 [SMILES: COCCn1c(-c2c[nH]c(-c3ccc(Cl)cc3)n2)nc2ccc(C(=O)Nc3cccnc3)cc21] | 49 | 473 | ¹H NMR (400 MHz, METHANOL-d₄) δ ppm 3.31 (s, 3H) 4.01-4.10 (m, 2 H) 5.14-5.23 (m, 2 H) 7.52-7.62 (m, 2 H) 7.78-7.86 (m, 1 H) 7.85- 7.92 (dd, J = 8.59, 0.5 Hz, 1 H) 8.00- 8.09 (m, 2 H) 8.16-8.24 (dd, J = 8.34, 1.52 Hz, 1 H) 8.32-8.39 (m, 1 H) 8.47-8.53 (dd, J = 5.31, 1.26 Hz, 1 H) 8.53-8.63 (m, 2 H) 9.31 (ld, J = 2.02 Hz, 1 H) |
| 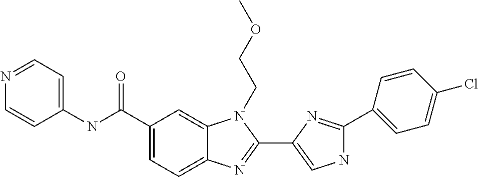 [SMILES: COCCn1c(-c2c[nH]c(-c3ccc(Cl)cc3)n2)nc2ccc(C(=O)Nc3ccncc3)cc21] | 50 | 473 | ¹H NMR (400 MHz, DMSO-d₆) δ ppm 3.21 (s, 3 H) 3.91 (s, 2 H) 5.08- 5.22 (m, 2 H) 7.63 (d, J = 8.59 Hz, 2 H) 7.80 (s, 1 H) 7.91-8.03 (m, 1 H) 8.09 (d, J = 8.59 Hz, 2 H) 8.24 (s, 1 H) 8.32 (d, J = 7.33 Hz, 2 H) 8.37 (d, J = 1.26 Hz, 1 H) 8.78 (d, J = 7.33 Hz, 2 H) 11.43-11.57 (m, 1 H) |
| 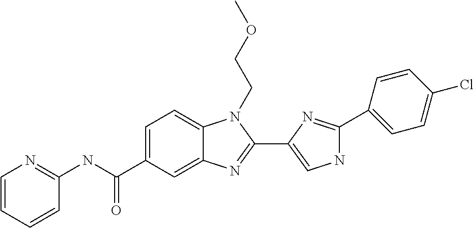 [SMILES: COCCn1c(-c2c[nH]c(-c3ccc(Cl)cc3)n2)nc2cc(C(=O)Nc3ccccn3)ccc21] | 51 | 473 | ¹H NMR (400 MHz, METHANOL-d₄) δ ppm 3.31 (s, 3H) 3.93 (s, 2 H) 5.05 (s, 2 H) 7.18-7.28 (m, 1 H) 7.46 (d, J = 8.84 Hz, 2 H) 7.90-7.98 (m, 4 |

### Example 52

### 2-[2-(4-chlorophenyl)-1H-imidazol-4-yl]-1-[2-(methyloxy)ethyl]-1H-imidazo[4,5-c]pyridine

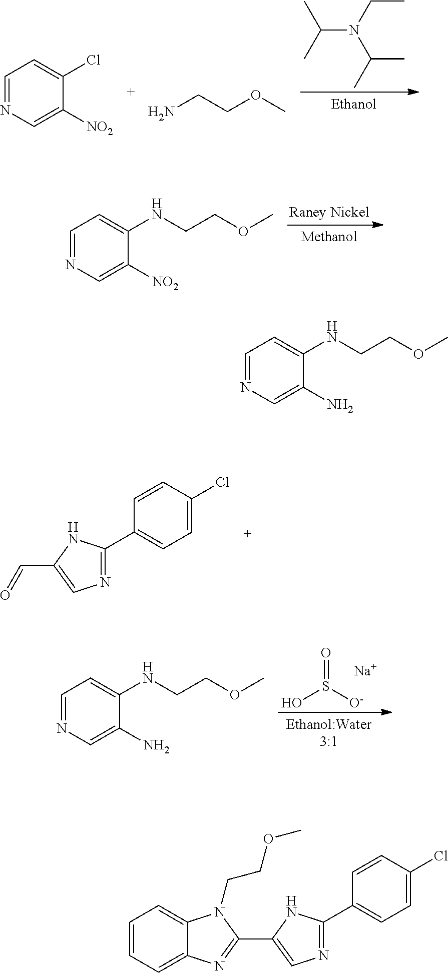

Step 1. N-[2-(methyloxy)ethyl]-3-nitro-4-pyridinamine: 4-chloro-3-nitropyridine (200 mg, 1.261 mmol) was dissolved in Ethanol (1500 μl) and DIEA (220 μl, 1.261 mmol) was added and added to a microwave vial. 2-(methyloxy)ethanamine (95 mg, 1.261 mmol) 100 was added and the vial was capped and heated at 150° C. for 19 minutes. LCMS confirms complete conversion to the product. The solvent was removed and the product was carried on without further purification. Quantitative yield was assumed.

Step 2. N-[2-(methyloxy)ethyl]-3,4-pyridinediamine: N-[2-(methyloxy)ethyl]-3-nitro-4-pyridinamine (200 mg, 1.014 mmol) was dissolved in Methanol (20 mL) and Raney Nickel (catalytic) was added and the reaction was stirred under a hydrogen atmosphere until complete by LCMS. Approx 1 hour. The reaction was then filtered and the solvent was removed under vacuum. Reaction was carried on without further purification. Quantitative yield was assumed.

Step 3. 2-[2-(4-chlorophenyl)-1H-imidazol-4-yl]-1-[2-(methyloxy)ethyl]-1H-imidazo[4,5-c]pyridine: 2-(4-chlorophenyl)-1H-imidazole-4-carbaldehyde (75 mg, 0.363 mmol) was dissolved in Ethanol (1000 μl) and N4-[2-(methyloxy)ethyl]-3,4-pyridinediamine (60.7 mg, 0.363 mmol) was added and added to a microwave vial along with Water (500 μl) and sodium bisulfite (76 mg, 0.726 mmol). The vial was capped and heated at 150° C. for 9 minutes. LCMS confirms complete conversion to the product. The solvent was removed and the product was purified by Prep HPLC. ¹H NMR (400 MHz, DMSO-d₆) δ ppm 3.18 (s, 3H) 3.87-3.94 (m, 2H) 5.21-5.28 (m, 2H) 7.63 (d, J=8.59 Hz, 2H) 8.08 (d, J=8.84 Hz, 2H) 8.22-8.27 (m, 1H) 8.38 (s, 1H) 8.62 (s, 1H) 9.36 (s, 1H); MS (m/z) 354.1 (M+H)⁺.

### Example 53

### 2-[2-(1,3-benzothiazol-5-yl)-1H-imidazol-4-yl]-1-[2-(methyloxy)ethyl]-1H-benzimidazole-6-carbonitrile hydrochloride

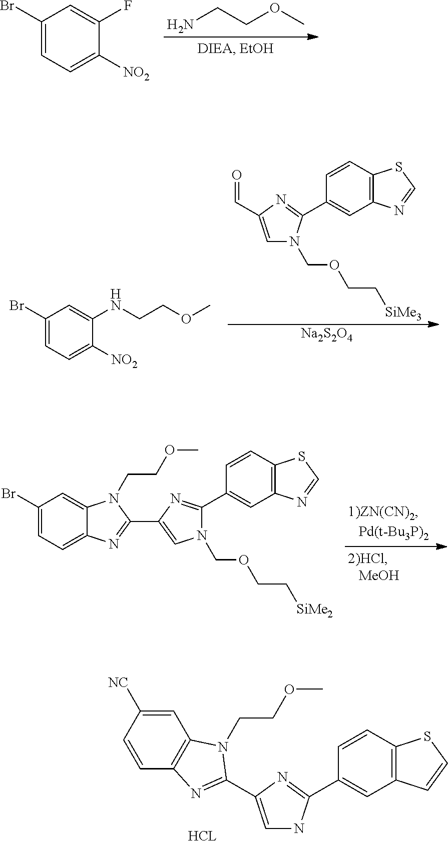

Step 1. 5-bromo-N-[2-(methyloxy)ethyl]-2-nitroaniline: 4-bromo-2-fluoro-1-nitrobenzene (0.3 g, 1.336 mmol), 2-(methyloxy)ethylamine (0.117 mL, 1.336 mmol), and DIEA (0.238 mL, 1.336 mmol) in Ethanol (5 mL) were irradiated by microwave at 100° C. for 30 min. The reaction mixture was partitioned between dichloromethane and brine. The aqueous layer was extracted with DCM twice. The combined DCM layers were dried over sodium sulfate, filtered, and concentrated. The residue was purified via Biotage (SNAP Cartridge KP Sil 25 g, 0-10% EtOAc/Hexane) to yield 5-bromo-N-[2-(methyloxy)ethyl]-2-nitroaniline (0.374 g, 1.286 mmol, 96% yield). ¹H NMR (400 MHz, CDCl₃) δ 8.22 (br. s., 1H), 8.04 (d, J=9.1 Hz, 1H), 7.05 (d, J=1.5 Hz, 1H), 6.78 (dd, 1H), 3.69 (t, 2H), 3.48 (t, 2H), 3.45 (s, 3H); MS (m/z) 275.1 (M+H)⁺.

Step 2. 5-[4-{6-bromo-1-[2-(methyloxy)ethyl]-1H-benzimidazol-2-yl]-1-({[2-(trimethylsilyl)ethyl]oxy}methyl)-1H-imidazol-2-yl}-1,3-benzothiazole: 2-(1,3-benzothiazol-5-yl)-1-({[2-(trimethylsilyl)ethyl]oxy}methyl)-1H-imidazole-4-carbaldehyde (0.394 g, 0.909 mmol), 5-bromo-N-[2-(methyloxy)ethyl]-2-nitroaniline (0.25 g, 0.909 mmol), and sodium hydrosulfite (0.558 g, 2.73 mmol) in Dimethyl Sulfoxide (DMSO) (6 mL) were irradiated by microwave at 100° C. for 60 min. The reaction mixture was partitioned between ethyl acetate and brine. The aqueous layer was extracted with EtOAc twice. The combined EtOAc layers were dried over sodium sulfate, filtered, and concentrated. The residue was purified via Biotage (SNAP Cartridge KP Sil 25 g, 0-50% EtOAc/Hexane) to yield 5-[4-{6-bromo-1-[2-(methyloxy)ethyl]-1H-benzimidazol-2-yl]-1-({[2-(trimethylsilyl)ethyl]oxy}methyl)-1H-imidazol-2-yl}-1,3-benzothiazole (0.1 g, 0.120 mmol, 13.18% yield). ¹H NMR (400 MHz, CDCl₃) δ 9.11 (s, 1H), 8.69 (s, 1H), 8.09-8.13 (m, 2H), 8.04 (d, J=8.4 Hz, 1H), 7.69 (d, J=1.5 Hz, 1H), 7.61 (d, J=8.6 Hz, 1H), 7.38 (dd, J=8.5 Hz, 1.8 Hz, 1H), 5.45 (s, 2H), 5.00 (t, J=5.5 Hz, 2H), 3.94 (t, 2H), 3.76 (t, J=8.1 Hz, 2H), 3.33 (s, 3H), 1.03 (t, 2H), 0.03 (s, 9H); MS (m/z) 584.4 (M+H)⁺.

Step 3. 2-[2-(1,3-benzothiazol-5-yl)-1H-imidazol-4-yl]-1-[2-(methyloxy)ethyl]-1H-benzimidazole-6-carbonitrile: To 5-[4-{6-bromo-1-[2-(methyloxy)ethyl]-1H-benzimidazol-2-yl}-1-({[2-(trimethylsilyl)ethyl]oxy}methyl)-1H-imidazol-2-yl]-1,3-benzothiazole (0.1 g, 0.171 mmol) in N,N-Dimethylformamide (DMF) (2 mL) was added zinc cyanide (0.020 g, 0.171 mmol). Deoxygenated for 10 mins, bis(tri-t-butylphosphine)palladium(0) (8.74 mg, 0.017 mmol) was added. The reaction mixture was irradiated by microwave at 150° C. for 1 hr. The reaction mixture was partitioned between ethyl acetate and brine, solid was filtered out. The aqueous layer was extracted with EtOAc twice, and combined EtOAc layers were concentrated. The residue and solid were dissolved in dimethyl sulfoxide, purified via reverse phase HPLC (Waters SunFire Prep C18 OBD 5 μm, 30×100 mm column, 20-70% acetonitrile/water 0.1% TFA, 40 ml/min, 10 min) to yield trifluoroacetic acid salt, then treated with 1N hydrochloric acid in methanol. Stirred at RT till white solid precipitated. The solid was filtered out and vacuum dried to yield hydrochloride of 2-[2-(1,3-benzothiazol-5-yl)-1H-imidazol-4-yl]-1-[2-(methyloxy)ethyl]-1H-benzimidazole-6-carbonitrile (0.034 g, 0.074 mmol, 43.3% yield). ¹H NMR (400 MHz, DMSO-d6) δ 9.51 (s, 1H), 8.79 (s, 1H), 8.38 (s, 1H), 8.35 (d, J=8.4 Hz, 2H), 8.23 (d, J=8.4 Hz, 1H), 7.82 (d, 1H), 7.72 (d, J=8.1 Hz, 1H), 5.15 (t, 2H), 3.91 (t, J=5.2 Hz, 2H), 3.23 (s, 3H); MS (m/z) 401 (M+H)⁺.

The following compounds were prepared with procedures analogous to that described in Example 53.

| Structure | Example | MS (m/z) | ¹H NMR |
|---|---|---|---|
| 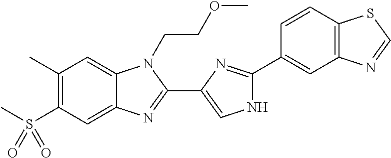 [SMILES: COCCn1c(-c2c[nH]c(-c3ccc4scnc4c3)n2)nc2cc(S(C)(=O)=O)c(C)cc21] | 54 | 468 | ¹H NMR (600 MHz, DMSO-d6) δ 13.41 (br. s., 1 H), 9.49 (s, 1 H), 8.74 (s, 1 H), 8.31 (d, J = 8.69 Hz, 1 H), 8.16-8.23 (m, 2 H), 8.13 (s, 1 H), 7.69 (s, 1 H), 5.08-5.15 (m, 2 H), 3.89 (t, J = 5.10 Hz, 2 H), 3.24 (s, 3 H), 3.22 (s, 3 H), 2.77 (s, 3 H) |
| Note: treated with 6M HCl in 1:1 Methanol/dichloromethane in step 3. | | | |
| 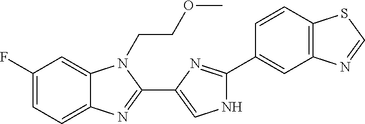 [SMILES: COCCn1c(-c2c[nH]c(-c3ccc4scnc4c3)n2)nc2ccc(F)cc21] | 55 | 394 | ¹H NMR (400 MHz, DMSO-d6) δ 13.36 (br. s., 1 H), 9.48 (s, 1 H), 8.75 (s, 1 H), 8.31 (d, J = 8.4 Hz, 1 H), 8.20 (d, 1 H), 8.09 (s, 1H), 7.60 (dd, J = 8.7 Hz, 4.9 Hz, 1 H), 7.52 (d, 1 H), 7.08 (td, J = 8.6 Hz, 2.2 Hz, 1H), 5.03 (s, 2 H), 3.87 (t, J = 5.5 Hz, 2 H), 3.23 (s, 3 H) |
| 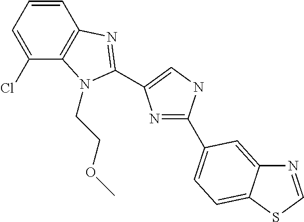 [SMILES: COCCn1c(-c2c[nH]c(-c3ccc4scnc4c3)n2)nc2cccc(Cl)c21] | 56 | 410 | |
| Note: prepared using 2-(1,3- benzothiazol-5-yl)-1H-imidazole-4- carbaldehyde in step 2; step 3 omitted. | | | |
| 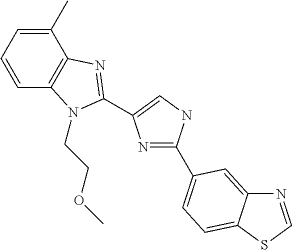 [SMILES: COCCn1c(-c2c[nH]c(-c3ccc4scnc4c3)n2)nc2c(C)cccc21] | 57 | 390 | ¹H NMR (400 MHz, CHLOROFORM-d) d ppm 2.79 (s, 3 H) 3.47 (br. s., 3 H) 4.00 (s, 2 H) 4.88- 5.14 (m, 2 H) 6.93-7.11 (m, 1 H) 7.14-7.27 (m, 2 H) 7.57-7.77 (m, 1 H) 7.87-8.30 (m, 2 H) 8.67 (s, 1 H) 9.09 (s, 1 H) |
| Note: prepared using 2-(1,3- benzothiazol-5-yl)-1H-imidazole-4- carbaldehyde in step 2; step 3 omitted. | | | |
| 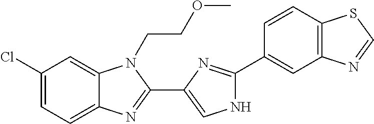 [SMILES: COCCn1c(-c2c[nH]c(-c3ccc4scnc4c3)n2)nc2ccc(Cl)cc21] | 58 | 410 | ¹H NMR (400 MHz, DMSO-d6) δ 13.33 (br. s., 1 H), 9.48 (s, 1 H), 8.73 (s, 1 H), 8.31 (d, J = 8.4 Hz, 1 H), 8.20 (dd, J = 8.5 Hz, 1.2 Hz, 1 H), 8.10 (s, 1H), 7.72 (d, 1 H), 7.58 (d, 1 H), 7.21 (dd, J = 8.6 Hz, 2 Hz, 1H), 5.07 (t, 2 H), 3.87 (t, J = 5.4 Hz, 2 H), 3.22 (s, 3 H) |
| Note: prepared using 2-(1,3- benzothiazol-5-yl)-1H-imidazole-4- carbaldehyde in step 2; step 3 omitted. | | | |
| 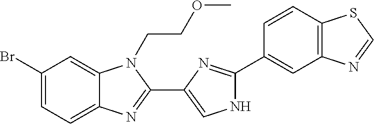 [SMILES: COCCn1c(-c2c[nH]c(-c3ccc4scnc4c3)n2)nc2ccc(Br)cc21] | 59 | 456 | ¹H NMR (400 MHz, DMSO-d6) δ 13.33 (br. s., 1 H), 9.48 (s, 1 H), 8.73 (s, 1 H), 8.31 (d, J = 8.5 Hz, 1 H), 8.19 (d, J = 8.4 Hz, 1 H), 8.11 (d, 1H), 7.85 (d, 1 H), 7.53 (d, 1 H), 7.32 (dd, J = 8.4 Hz, 1.7 Hz, 1H), 5.07 (t, 2 H), 3.87 (t, J = 5.4 Hz, 2 H), 3.22 (s, 3 H) |
| Note: prepared using 2-(1,3- benzothiazol-5-yl)-1H-imidazole-4- carbaldehyde in step 2; step 3 omitted. | | | |
| 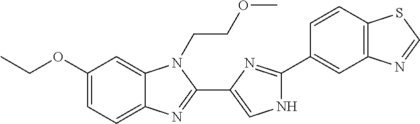 [SMILES: CCOc1ccc2nc(-c3c[nH]c(-c4ccc5scnc5c4)n3)n(CCOC)c2c1] | 60 | 420 | ¹H NMR (400 MHz, DMSO-d6) δ 13.22 (br. s., 1 H), 9.47 (s, 1 H), 8.73 (s, 1 H), 8.31 (d, J = 8.6 Hz, 1 H), 8.19 (d, J = 8.3 Hz, 1 H), 7.99 (s, 1H), 7.45 (d, J = 8.5 Hz, 1 H), 7.14 (s, 1 H), 6.81 (d, J = 8.8 Hz, 1H), 5.01 (s, 2 H), 4.10 (q, 2H), 3.85 (t, J = 5.2 Hz, 2 H), 3.25 (s, 3H), 1.38 (t, J = 7 Hz, 3 H) |
| Note: prepared using 2-(1,3- benzothiazol-5-yl)-1H-imidazole-4- carbaldehyde in step 2; step 3 omitted. | | | |
| 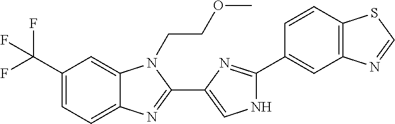 [SMILES: COCCn1c(-c2c[nH]c(-c3ccc4scnc4c3)n2)nc2ccc(C(F)(F)F)cc21] | 61 | 444 | ¹H NMR (400 MHz, DMSO-d6) δ 13.39 (br. s., 1 H), 9.49 (s, 1 H), 8.75 (s, 1 H), 8.32 (d, J = 8.3 Hz, 1 H), 8.20 (d, J = 10.9 Hz,1 H), 8.19 (s, 1H), 8.03 (s, 1H), 7.76 (d, 1 H), 7.51 (d, 1 H), 5.18 (t, 2 H), 3.90 (t, J = 5.2 Hz, 2 H), 3.22 (s, 3 H) |
| Note: prepared using 2-(1,3- benzothiazol-5-yl)-1H-imidazole-4- carbaldehyde in step 2; step 3 omitted. | | | |

### Example 62

### 2-[2-(1,3-benzothiazol-5-yl)-1H-imidazol-4-yl]-N,N,6-trimethyl-1-[2-(methyloxy)ethyl]-1H-benzimidazole-5-carboxamide

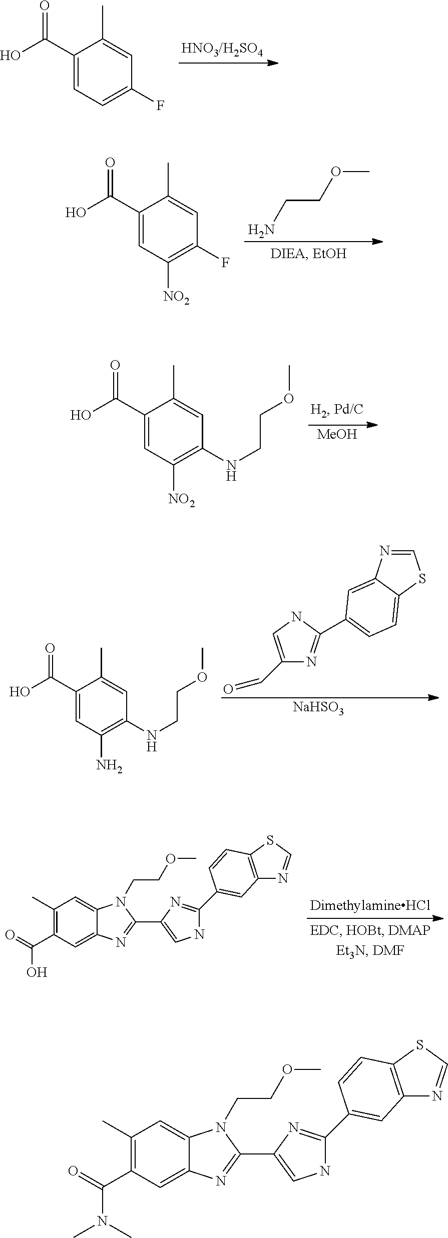

Step 1. 4-fluoro-2-methyl-5-nitrobenzoic acid: nitric acid (0.4 ml, 8.06 mmol) was added to a stirred solution of 4-fluoro-2-methylbenzoic acid (1 g, 6.49 mmol) in sulfuric acid (4.7 ml, 88 mmol) at 0° C. Stirred for 45 mins, diluted with ethyl acetate and poured into ice water. The aqueous phase was extracted with EtOAc twice, the combined EtOAc layers were washed with brine, dried over sodium sulfate. The EtOAc layer was concentrated, and purified via Biotage (SNAP Cartridge KP Sil 25 g, 0-5% Methanol/Dichloromethane, 1% Acetic acid) to yield 4-fluoro-2-methyl-5-nitrobenzoic acid (1.08 g, 5.42 mmol, 84 yield). ¹H NMR (600 MHz, CDCl₃) δ 8.85 (d, J=7.55 Hz, 1H), 7.24 (d, J=11.33 Hz, 1H), 2.76 (s, 3H); MS (m/z) 200.1 (M+H)⁺.

Step 2. 2-methyl-4-{[2-(methyloxy)ethyl]amino}-5-nitrobenzoic acid: 4-fluoro-2-methyl-5-nitrobenzoic acid (1.08 g, 5.42 mmol), 2-(methyloxy)ethylamine (0.476 mL, 5.42 mmol), and DIEA (0.967 mL, 5.42 mmol) in Ethanol (5 mL) were heated at 100° C., stirred for 3 hours. The reaction was partitioned between ethyl acetate and brine, foam formed. The aqueous layer was separated and adjusted PH<4 with 1N hydrochloric acid, then extracted with EtOAc. The combined EtOAc layers were dried over sodium sulfate, filtered, concentrated, air-dried to yield 2-methyl-4-{[2-(methyloxy)ethyl]amino}-5-nitrobenzoic acid (1.2 g, 4.72 mmol, 87% yield). MS (m/z) 255.2 (M+H)⁺.

Step 3. 5-amino-2-methyl-4-{[2-(methyloxy)ethyl]amino}benzoic acid: The hydrogenation of 2-methyl-4-{[2-(methyloxy)ethyl]amino}-5-nitrobenzoic acid (0.8 g, 3.15 mmol) in methanol (100 mL) was carried out under hydrogen balloon atmosphere in the presence of palladium on carbon (0.067 g, 0.031 mmol). The catalyst was filtered through a Celite pad and washed with methanol, and the filtrate solution was concentrated in vacuo. The residue was purified via Biotage (SNAP Cartridge KP Sil 25 g, 0-50% EtOAc/Hexane, 0-5% Methanol/dichloromethane) to yield 5-amino-2-methyl-4-{[2-(methyloxy)ethyl]amino}benzoic acid (0.544 g, 2.013 mmol, 64.0% yield). ¹H NMR (400 MHz, CDCl₃) δ 7.51 (s, 1H), 7.28 (s, 1H), 6.43 (s, 1H), 3.68 (t, 2H), 3.43 (s, 3H), 3.38 (t, 2H), 2.59 (s, 3H); MS (m/z) 225.2 (M+H)⁺.

Step 4. 2-[2-(1,3-benzothiazol-5-yl)-1H-imidazol-4-yl]-6-methyl-1-[2-(methyloxy)ethyl]-1H-benzimidazole-5-carboxylic acid: 5-amino-2-methyl-4-{[2-(methyloxy)ethyl]amino}benzoic acid (0.359 g, 1.601 mmol), 2-(1,3-benzothiazol-5-yl)-1H-imidazole-4-carbaldehyde (0.408 g, 1.601 mmol), and sodium bisulfite (0.333 g, 3.20 mmol) in Ethanol (10 mL), Water (2.000 mL) were heated at 100° C. The reaction mixture was partitioned between 1N sodium hydroxide solution and ethyl acetate. The EtOAc layer was extracted with 1N NaOH twice. The combined aqueous layers was adjusted PH<4 with hydrochloric acid solution, solid precipitated. The solid was filtered and vacuum dried to yield 2-[2-(1,3-benzothiazol-5-yl)-1H-imidazol-4-yl]-6-methyl-1-[2-(methyloxy)ethyl]-1H-benzimidazole-5-carboxylic acid (0.5 g, 0.911 mmol, 56.9% yield). ¹H NMR (400 MHz, DMSO-d6) δ 9.50 (s, 1H), 8.77 (s, 1H), 8.34 (d, J=8.6 Hz, 1H), 8.28 (s, 1H), 8.22 (dd, J=8.4 Hz, 1.5 Hz, 1H), 8.14 (s, 1H), 7.67 (s, 1H), 5.06 (t, 2H), 3.90 (t, J=5.5 Hz, 2H), 2.69 (s, 3H); MS (m/z) 434.4 (M+H)⁺.

Step 5. 2-[2-(1,3-benzothiazol-5-yl)-1H-imidazol-4-yl]-N,N,6-trimethyl-1-[2-(methyloxy)ethyl]-1H-benzimidazole-5-carboxamide: 2-[2-(1,3-benzothiazol-5-yl)-1H-imidazol-4-yl]-6-methyl-1-[2-(methyloxy)ethyl]-1H-benzimidazole-5-carboxylic acid (0.127 g, 0.231 mmol), EDC (0.133 g, 0.694 mmol), HOBT (0.106 g, 0.694 mmol), HOBT (0.106 g, 0.694 mmol), DMAP (1.414 mg, 0.012 mmol), TEA (0.097 mL, 0.694 mmol), and dimethylamine hydrochloride (0.038 g, 0.463 mmol) in N,N-Dimethylformamide (DMF) (3 mL) were stirred at RT. The reaction mixture was partitioned between ethyl acetate and brine. The aqueous layer was extracted with EtOAc twice, and combined EtOAc layers were concentrated. The residue was dissolved in dimethyl sulfoxide, purified via reverse phase HPLC (Waters SunFire Prep C18 OBD 5 μm, 30×100 mm column, 20-50% acetonitrile/water 0.1% TFA, 40 ml/min, 10 min) to yield trifluoroacetic acid salt. The salt was basified with saturated sodium carbonate, then extracted with chloroform. The chloroform layer was dried over sodium sulfate, filtered, concentrated and vacuum dried to yield 2-[2-(1,3-benzothiazol-5-yl)-1H-imidazol-4-yl]-N,N,6-trimethyl-1-[2-(methyloxy)ethyl]-1H-benzimidazole-5-carboxamide (0.059 g, 0.123 mmol, 53.4% yield). ¹H NMR (400 MHz, DMSO-d6) δ 13.32 (br. s., 1H), 9.48 (s, 1H), 8.75 (s, 1H), 8.31 (d, J=8.6 Hz, 1H), 8.20 (d, 1H), 8.06 (s, 1H), 7.50 (s, 1H), 7.35 (s, 1H), 5.03 (s, 2H), 3.88 (t, J=5.5 Hz, 2 H), 3.24 (s, 3H), 3.04 (s, 3H), 2.79 (s, 3H), 2.32 (s, 3H); MS (m/z) 461.4 (M+H)⁺.

The following compounds were prepared with procedures analogous to that described in Step 5 of Example 62.

| Structure | Example | MS (m/z) | ¹H NMR |
|---|---|---|---|
| 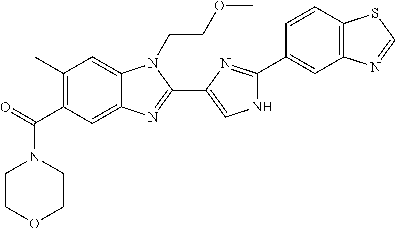 [SMILES: COCCn1c(-c2c[nH]c(-c3ccc4scnc4c3)n2)nc2cc(C(=O)N3CCOCC3)c(C)cc21] | 63 | 503.5 | ¹H NMR (400 MHz, DMSO-d6) δ 13.4 (br. s., 1 H), 9.49 (s, 1 H), 8.75 (s, 1 H), 8.32 (d, J = 8.3 Hz, 1 H), 8.21 (dd, J = 8.3 Hz, 1.3 Hz, 1 H), 8.13 (s, 1 H), 7.57 (s, 1 H), 7.42 (s, 1 H), 5.04 (s, 2 H), 3.88 (t, J = 5.6 Hz, 2 H), 3.69 (s, 4 H), 3.51 (d, 2 H), 3.25 (s, 3 H), 3.18 (s, 2 H), 2.37 (s, 3 H) |
| 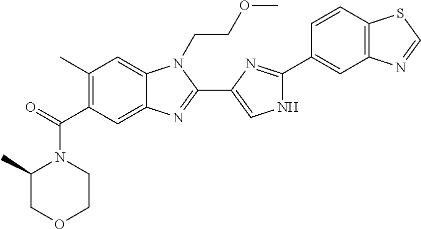 [SMILES: COCCn1c(-c2c[nH]c(-c3ccc4scnc4c3)n2)nc2cc(C(=O)N3CCOC[C@H]3C)c(C)cc21] | 64 | 517.5 | ¹H NMR (400 MHz, DMSO-d6) δ 13.4 (br. s., 1 H), 9.49 (s, 1 H), 8.75 (s, 1 H), 8.31 (d, J = 8.6 Hz, 1 H), 8.20 (d, J = 8.3 Hz, 1 H), 8.09 (br. s., 1 H), 7.53 (br. s., 1 H), 7.39 (br. s., 1 H), 5.03 (br. s., 2 H), 3.89 (t, 2 H), 3.70 (br. s., 2 H), 3.49 (br. s., 1 H), 3.25 (s, 3 H), 3.17 (s, 1 H), 3.01-3.10 (m, 1 H), 2.39 (s, 2 H), 2.30 (s, 1 H), 1.39-1.19 (m, 3 H), 1.08-1.18 (m, 1 H), 0.81-0.90 (m, 1 H) |
| 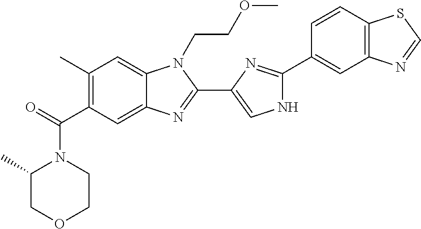 [SMILES: COCCn1c(-c2c[nH]c(-c3ccc4scnc4c3)n2)nc2cc(C(=O)N3CCOC[C@@H]3C)c(C)cc21] | 65 | 517.5 | ¹H NMR (400 MHz, DMSO-d6) δ 13.4 (br. s., 1 H), 9.49 (s, 1 H), 8.75 (s, 1 H), 8.31 (d, J = 8.6 Hz, 1 H), 8.20 (d, J= 8.3 Hz, 1 H), 8.09 (br. s., 1 H), 7.53 (br. s., 1 H), 7.39 (br. s., 1 H), 5.03 (br. s., 2 H), 3.89 (t, 2 H), 3.70 (br. s., 2 H), 3.49 (br. s., 1 H), 3.25 (s, 3 H), 3.17 (s, 1 H), 3.01-3.10 (m, 1 H), 2.39 (s, 2 H), 2.30 (s, 1 H), 1.39-1.19 (m, 3 H), 1.08-1.18 (m, 1 H), 0.81-0.90 (m, 1 H) |
| 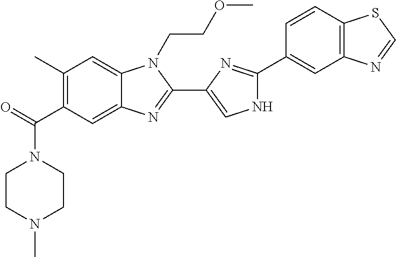 [SMILES: COCCn1c(-c2c[nH]c(-c3ccc4scnc4c3)n2)nc2cc(C(=O)N3CCN(C)CC3)c(C)cc21] | 66 | 516.5 | ¹H NMR (400 MHz, DMSO-d6) δ 13.3 (br. s., 1 H), 9.48 (s, 1 H), 8.73 (s, 1 H), 8.31 (d, J = 8.3 Hz, 1 H), 8.19 (d, J = 8.6 Hz, 1 H), 8.07 (d, J = 1.8 Hz, 1 H), 7.50 (s, 1 H), 7.35 (s, 1 H), 5.04 (br. s., 2 H), 3.88 (t, J = 5.6 Hz, 2 H), 3.69 (br. s., 1 H), 3.25 (s, 3 H), 3.22 (br. s., 3 H), 2.35 (s, 5 H), 2.29 (br. s., 3 H) |

The following compounds 67, 69 were prepared with procedures analogous to that described in Step 2-4 of Example 62. Compound 68 was prepared from hydrolysis of compound 66.

| Structure | Example | MS (m/z) | ¹H NMR |
|---|---|---|---|
| 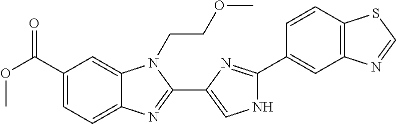 [SMILES: COCCn1c(-c2c[nH]c(-c3ccc4scnc4c3)n2)nc2ccc(C(=O)OC)cc21] | 67 | 434.4 | ¹H NMR (400 MHz, DMSO-d6) δ 9.51 (s, 1 H), 8.78 (d, J = 1 Hz, 1 H), 8.39-8.34 (m, 3 H), 8.22 (dd, J = 8.5 Hz, 1.5 Hz, 1 H), 7.98 (dd, 1 H), 7.77 (d, J = 8.4 Hz, 1 H), 5.17 (t, J = 4.9 Hz, 2 H), 3.92 (s, 6 H), 3.21 (s, 3 H) |
| 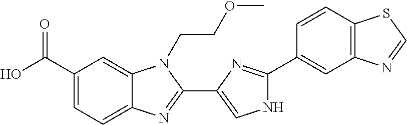 [SMILES: COCCn1c(-c2c[nH]c(-c3ccc4scnc4c3)n2)nc2ccc(C(=O)O)cc21] | 68 | 420.4 | ¹H NMR (400 MHz, DMSO-d6) δ 9.51 (s, 1 H), 8.78 (d, J = 1.3 Hz, 1 H), 8.38-8.34 (m, 3 H), 8.22 (dd, J = 8.4 Hz, 1.6 Hz, 1 H), 7.98 (dd, 1 H), 7.76 (d, 1 H), 5.16 (t, 2 H), 3.93 (t, J = 5.2 Hz, 3 H), 3.22 (s, 3 H) |
| 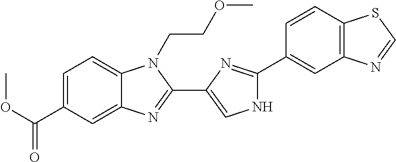 [SMILES: COCCn1c(-c2c[nH]c(-c3ccc4scnc4c3)n2)nc2cc(C(=O)OC)ccc21] | 69 | 434.4 | ¹H NMR (400 MHz, DMSO-d6) δ 13.4 (br. s., 1 H), 9.48 (s, 1 H), 8.75 (s, 1 H), 8.31 (d, J = 8.3 Hz, 3 H), 8.21-8.17 (m, 3 H), 7.86 (dd, J = 8.4 Hz, 1.4 Hz, 1 H), 7.71 (d, 1 H), 5.11 (t, J= 5.1 Hz, 2 H), 3.90 (t, 3 H), 3.21 (s, 3 H) |

### Example 70

### 2-{2-[2-(1,3-benzothiazol-5-yl)-1H-imidazol-4-yl]-1-[2-(methyloxy)ethyl]-1H-benzimidazol-5-yl}-2-propanol

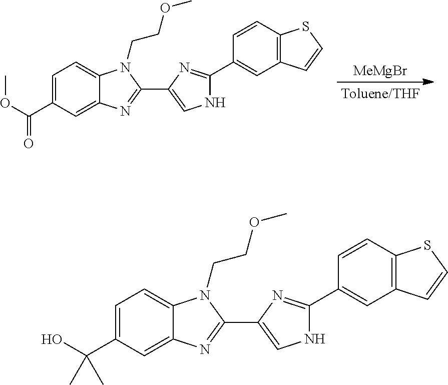 [SMILES: "MeMgBr" ; COCCn1c(-c2c[nH]c(-c3ccc4sccc4c3)n2)nc2cc(C(=O)OC)ccc21 ; COCCn1c(-c2c[nH]c(-c3ccc4sccc4c3)n2)nc2cc(C(C)(C)O)ccc21]

To a suspension of methyl 2-[2-(1,3-benzothiazol-5-yl)-1H-imidazol-4-yl]-1-[2-(methyloxy)ethyl]-1H-benzimidazole-5-carboxylate (0.15 g, 0.308 mmol) in Tetrahydrofuran (THF) (1 mL), Toluene (3.00 mL) at 0° C. was added Methylmagnesium bromide (1.100 mL, 1.540 mmol) in Toluene/THF (3:1), warmed up to RT while stirring. The reaction mixture was concentrated, then partitioned between dichloromethane and brine. The aqueous layer was extracted with DCM twice and the combined DCM layers were dried over sodium sulfate, filtered, and concentrated. The crude was dissolved in 2 mL dimethyl sulfoxide, purified via reverse phase HPLC (Xbridge C18, 30×150 mm, 10-50% acetonitrile/water 0.1% ammonium hydroxide, 50 ml/min, 10 mins) to yield 2-{2-[2-(1,3-benzothiazol-5-yl)-1H-imidazol-4-yl]-1-[2-(methyloxy)ethyl]-1H-benzimidazol-5-yl}-2-propanol (0.036 g, 0.081 mmol, 26.4% yield). ¹H NMR (400 MHz, DMSO-d6) δ 13.25 (br. s., 1H), 9.48 (s, 1H), 8.74 (s, 1H), 8.30 (d, J=8.3 Hz, 1H), 8.20 (d, J=7.8 Hz, 1H), 8.03 (s, 1H), 7.66 (s, 1H), 7.49 (d, 1H), 7.35 (dd, J=8.4 Hz, 1.5 Hz, 1H), 5.03 (br. s., 2 H), 4.99 (s, 1H), 3.86 (t, J=5.6 Hz, 2H), 3.24 (s, 3H), 1.50 (s, 6H); MS (m/z) 434 (M+H)⁺.

### Example 71

### 5-(4-{4-fluoro-1-[2-(methyloxy)ethyl]-1H-benzimidazol-2-yl}-1H-imidazol-2-yl)-1,3-benzothiazole trifluoroacetate

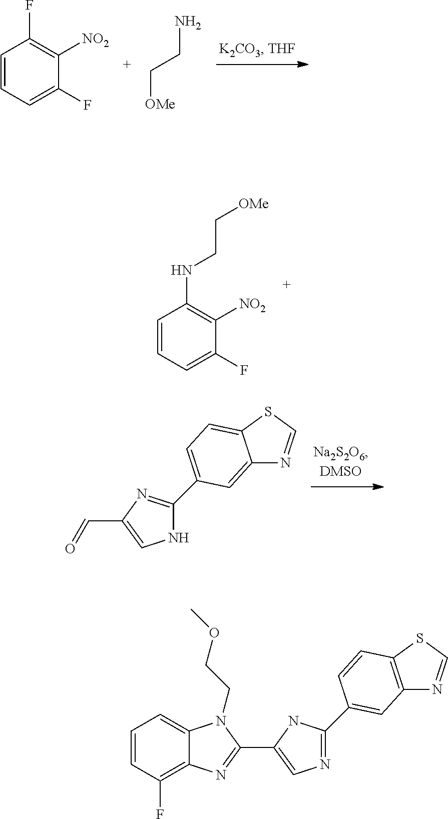 [SMILES: O=[N+]([O-])c1c(F)cccc1F ; COCCN ; COCCNc1cccc(F)c1[N+](=O)[O-] ; O=Cc1c[nH]c(-c2ccc3scnc3c2)n1 ; COCCn1c(-c2cnc(-c3ccc4scnc4c3)[nH]2)nc2c(F)cccc21]

Step 1. (3-fluoro-2-nitrophenyl)[2-(methyloxy)ethyl]amine: To a solution of 2,6-difluorodinitrobenzene (4 g, 24.64 mmol) in Tetrahydrofuran (THF) (80 ml) at rt was added 2-methoxyethlyamine (2.164 ml, 24.64 mmol). This was stirred at rt for 80 min, at which time, LCMS showed nearly half complete. Allowed to stir for 8 hr. LCMS showed 12% sm, 85% product, and 3% double addition product. Diluted with Et2O and water, separated layers and back-extracted with Et2O twice. Washed with Brine and dried on MgSO4. Filtered and concentrated. Purified via Biotage FCC (40 g SNAP column, 0-10% EtOAC/hex); baseline separation was achieved. Combined an concentrated product fractions to provide (3-fluoro-2-nitrophenyl)[2-(methyloxy)ethyl]amine (3.76 g, 17.55 mmol, 71.2% yield) as a bright orange oil. MS (m/z) 215.0 (M+H)⁺.

Step 2. 5-(4-{4-fluoro-1-[2-(methyloxy)ethyl]-1H-benzimidazol-2-yl}-1H-imidazol-2-yl)-1,3-benzothiazole: A suspension of sodium dithionate (144 mg, 0.700 mmol) in dimethyl sulfoxide (DMSO) (1167 μl) was heated to 80° C. This was stirred for 5 min at which time (3-fluoro-2-nitrophenyl)[2-(methyloxy)ethyl]amine (50 mg, 0.233 mmol) and 2-(1,3-benzothiazol-5-yl)-1H-imidazole-4-carbaldehyde (53.5 mg, 0.233 mmol) were simultaneously added. This was stirred at 80° C. overnight. LCMS showed mostly product. The crude reaction mixture was filtered through an acrodisc and purified on a Mass directed Agilent HPLC system, on a Waters Sunfire 30×150 mm column with a gradient of 26-60% acetonitrile/water (0.1% TFA) providing the title compound: TFA salt of 5-(4-{4-fluoro-1-[2-(methyloxy)ethyl]-1H-benzimidazol-2-yl}-1H-imidazol-2-yl)-1,3-benzothiazole (30.1 mg, 0.059 mmol, 25.4% yield). ¹H NMR (400 MHz, DMSO-d₆) ^{TM} ppm 9.49 (s, 1H), 8.76 (s, 1H), 8.32 (d, J=8.3 Hz, 1H), 8.17-8.25 (m, 3H), 7.50 (d, J=8.0 Hz, 1H), 7.24 (br. s., 1H), 7.00-7.11 (m, 1H), 5.07 (t, J=5.4 Hz, 2H), 3.89 (t, J=5.5 Hz, 2H), 3.22 (s, 3H); MS (m/z): 394.2 (M+H)⁺.

### Example 72

### 2-[2-(1,3-benzothiazol-5-yl)-1H-imidazol-4-yl]-3-[2-(methyloxy)ethyl]-3H-imidazo[4,5-b]pyridine

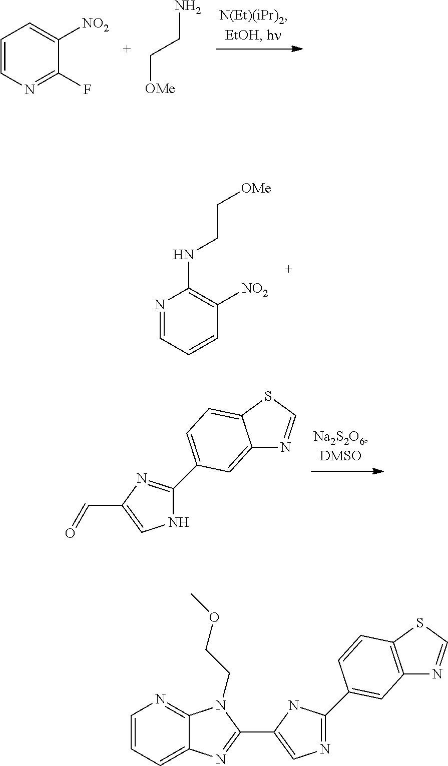 [SMILES: O=[N+]([O-])c1cccnc1F ; COCCN ; COCCNc1ncccc1[N+](=O)[O-] ; O=Cc1c[nH]c(-c2ccc3scnc3c2)n1 ; COCCn1c(-c2cnc(-c3ccc4scnc4c3)[nH]2)nc2cccnc21]

Step 1. 2-fluoro-3-nitropyridine: Added DIEA (738 μl, 4.22 mmol) to a solution of 3-fluoro-2-nitropyridine (500 mg, 3.52 mmol) and 2-(methoxy)ethylamine (333 μl, 3.87 mmol) in Ethanol (2448 μl) and heated in a microwave to 150° C. for eight minutes. LCMS showed the reaction was complete. Concentrated the reaction and used the material without purification. MS (m/z) 198.1 (M+H)⁺.

Step 2. 2-[2-(1,3-benzothiazol-5-yl)-1H-imidazol-4-yl]-3-[2-(methyloxy)ethyl]-3H-imidazo[4,5-b]pyridine: Added sodium dithionite (265 mg, 1.521 mmol) to Dimethyl Sulfoxide (DMSO) (2536 μl) and heated to 80° C. for five minutes. Then added N-[2-(methyloxy)ethyl]-3-nitro-2-pyridinamine (100 mg, 0.507 mmol) and 2-(1,3-benzothiazol-5-yl)-1H-imidazole-4-carbaldehyde (116 mg, 0.507 mmol) and continued heating at 80° C. overnight. Poured the reaction onto Celite and ran a reverse phase C18 Isco column using a gradient of 10-100% CH3CN/water. Isolated the desired fractions and concentrated to give 2-[2-(1,3-benzothiazol-5-yl)-1H-imidazol-4-yl]-3-[2-(methyloxy)ethyl]-3H-imidazo[4,5-b]pyridine (27 mg, 0.072 mmol, 14.14% yield). ¹H NMR (400 MHz, DMSO-d₆) ^{TM} 13.39 (br. s., 1H), 9.49 (s, 1H), 8.75 (d, J=1.0 Hz, 1H), 8.32-8.35 (m, 1H), 8.31 (s, 1H), 8.21 (dd, J=8.3, 1.5 Hz, 1H), 8.18 (s, 1H), 7.99 (dd, J=8.0, 1.4 Hz, 1H), 7.27 (dd, J=7.8, 4.8 Hz, 1H), 5.15 (t, J=6.1 Hz, 2H), 3.91 (t, J=6.1 Hz, 2H), 3.26 (s, 3H); MS (m/z): 377.1 (M+H)⁺.

The following compounds were prepared with a procedure analogous to that described in Example 79

| Structure | Example | MS (m/z) | ¹H NMR/Notes |
|---|---|---|---|
| 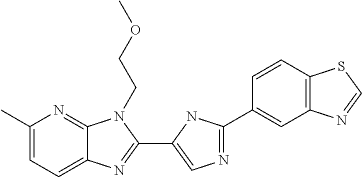 [SMILES: COCCn1c(-c2cnc(-c3ccc4scnc4c3)[nH]2)nc2ccc(C)nc21] | 73 | 391.2 | ¹H NMR (400 MHz, DMSO-d₆) ™ ppm 13.34 (br. s., 1 H), 9.49 (s, 1 H), 8.74 (d, J = 1.3 Hz, 1 H), 8.32 (d, J = 8.3 Hz, 1 H), 8.20 (dd, J = 8.3, 1.3 Hz, 1 H), 8.12 (d, J = 2.0 Hz, 1 H), 7.86 (d, J = 8.1 Hz, 1 H), 7.13 (d, J = 8.1 Hz, 1 H), 5.11 (t, J = 6.1 Hz, 2 H), 3.88 (t, J = 6.2 Hz, 2 H), 3.26 (s, 3 H), 2.60 (s, 3 H) |
| 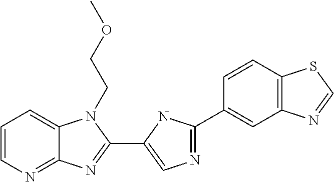 [SMILES: COCCn1c(-c2cnc(-c3ccc4scnc4c3)[nH]2)nc2ncccc21] | 74 | 377.2 | ¹H NMR (400 MHz, DMSO-d₆) ™ ppm 13.40 (br. s., 1 H), 9.49 (s, 1 H), 8.75 (s, 1 H), 8.37 (d, J = 4.5 Hz, 1 H), 8.31 (d, J = 8.3 Hz, 1 H), 8.17-8.24 (m, 2 H), 8.01 (d, J = 7.8 Hz, 1 H), 7.18-7.28 (m, 1 H), 5.11 (br. s., 2 H), 3.90 (t, J = 4.7 Hz, 2 H), 3.22 (s, 3 H) |
| 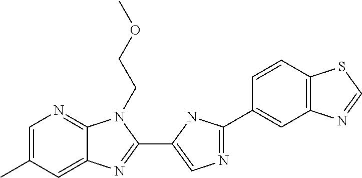 [SMILES: COCCn1c(-c2cnc(-c3ccc4scnc4c3)[nH]2)nc2cc(C)cnc21] | 75 | 391.3 | ¹H NMR (400 MHz, DMSO-d₆) ™ ppm 13.36 (br. s., 1 H), 9.48 (s, 1 H), 8.74 (s, 1 H), 8.31 (d, J = 8.3 Hz, 1 H), 8.20 (d, J = 8.3 Hz, 1 H), 8.15 (d, J = 4.5 Hz, 2 H), 7.79 (s, 1 H), 5.12 (t, J = 5.9 Hz, 2 H), 3.88 (t, J = 5.9 Hz, 2 H), 3.25 (s, 3 H), 2.43 (s, 3 H) |
| 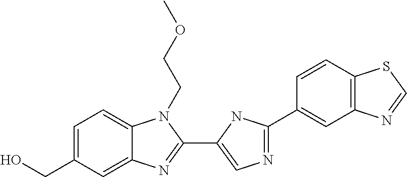 [SMILES: COCCn1c(-c2cnc(-c3ccc4scnc4c3)[nH]2)nc2cc(CO)ccc21] | 76 | 406.1 | ¹H NMR (500 MHz, DMSO-d₆) ™ ppm 13.29 (br. s., 1 H), 9.48 (s, 1 H), 8.73 (s, 1 H), 8.31 (d, J = 8.3 Hz, 1 H), 8.20 (s, 1 H), 8.08 (s, 1 H), 7.50-7.60 (m, 2 H), 7.19 (d, J = 8.3 Hz, 1 H), 5.13 (br. s., 1 H), 5.05 (t, J = 5.2 Hz, 2 H), 4.59 (d, J = 5.3 Hz, 2 H), 3.87 (t, J = 5.4 Hz, 2 H), 3.23 (s, 3 H) |
| 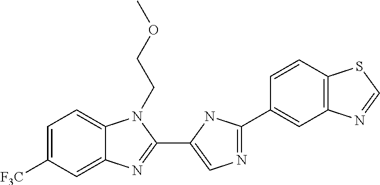 [SMILES: COCCn1c(-c2cnc(-c3ccc4scnc4c3)[nH]2)nc2cc(C(F)(F)F)ccc21] | 77 | 444.2 | ¹H NMR (400 MHz, DMSO-d₆) ™ ppm 13.39 (br. s., 1 H), 9.49 (s, 1 H), 8.75 (d, J = 1.3 Hz, 1 H), 8.32 (d, J = 8.6 Hz, 1 H), 8.20 (dd, J = 8.5, 1.4 Hz, 1 H), 8.18 (d, J = 1.5 Hz, 1 H), 7.92 (s, 1 H), 7.82 (d, J = 8.3 Hz, 1 H), 7.54 (dd, J = 8.6, 1.3 Hz, 1 H), 5.14 (t, J = 5.3 Hz, 2 H), 3.91 (t, J = 5.3 Hz, 2 H), 3.22 (s, 3 H) |
| 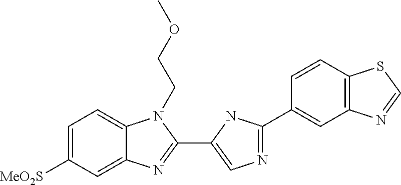 [SMILES: COCCn1c(-c2cnc(-c3ccc4scnc4c3)[nH]2)nc2cc(S(C)(=O)=O)ccc21] | 78 | 454.1 | ¹H NMR (500 MHz, DMSO-d₆) ™ ppm 13.42 (br. s., 1 H), 9.49 (s, 1 H), 8.75 (s, 1 H), 8.32 (d, J = 8.6 Hz, 1 H), 8.17-8.25 (m, 2 H), 8.11 (s, 1 H), 7.86 (m, J = 8.6 Hz, 1 H), 7.77 (d, J = 8.6 Hz, 1 H), 5.15 (t, J = 5.2 Hz, 2 H), 3.91 (t, J = 5.2 Hz, 2 H), 3.24 (s, 3 H), 3.22 (s, 3 H) |
| 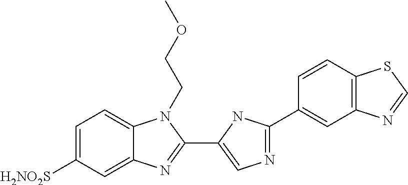 [SMILES: COCCn1c(-c2cnc(-c3ccc4scnc4c3)[nH]2)nc2cc(S(N)(=O)=O)ccc21] | 79 | 455.1 | ¹H NMR (400 MHz, DMSO-d₆) ™ ppm 13.39 (br. s., 1 H), 9.49 (s, 1 H), 8.74 (s, 1 H), 8.32 (d, J = 8.6 Hz, 1 H), 8.17-8.23 (m, 2 H), 8.02 (d, J = 1.3 Hz, 1 H), 7.78 (m, J = 8.6 Hz, 1 H), 7.70 (dd, J = 8.6, 1.5 Hz, 1 H), 7.27 (s, 2 H), 5.13 (t, J = 5.2 Hz, 2 H), 3.90 (t, J = 5.4 Hz, 2 H), 3.22 (s, 3 H) |
| 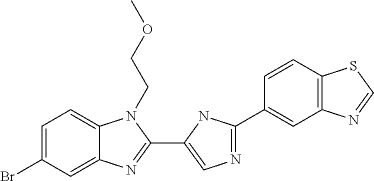 [SMILES: COCCn1c(-c2cnc(-c3ccc4scnc4c3)[nH]2)nc2cc(Br)ccc21] | 80 | 454.0 | ¹H NMR (400 MHz, DMSO-d₆) ™ ppm 13.35 (br. s., 1 H), 9.48 (s, 1 H), 8.73 (s, 1 H), 8.31 (d, J = 8.3 Hz, 1 H), 8.19 (d, J = 7.6 Hz, 1 H), 8.12 (s, 1 H), 7.76 (s, 1 H), 7.59 (d, J = 8.6 Hz, 1 H), 7.35 (dd, J = 8.6, 1.5 Hz, 1 H), 5.07 (t, J = 5.1 Hz, 2 H), 3.88 (t, J = 5.3 Hz, 2 H), 3.22 (s, 3 H) |
| 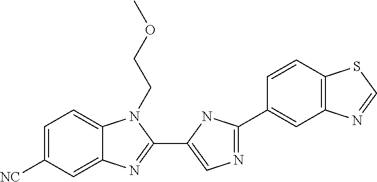 [SMILES: COCCn1c(-c2cnc(-c3ccc4scnc4c3)[nH]2)nc2cc(C#N)ccc21] | 81 | 401.2 | ¹H NMR (400 MHz, DMSO-d₆) ™ ppm 13.41 (br. s., 1 H), 9.49 (s, 1 H), 8.74 (s, 1 H), 8.32 (d, J = 8.6 Hz, 1 H), 8.16-8.23 (m, 2 H), 8.11 (s, 1 H), 7.81 (d, J = 8.6 Hz, 1 H), 7.62 (d, J = 8.3 Hz, 1 H), 5.14 (t, J = 4.9 Hz, 2 H), 3.90 (t, J = 5.1 Hz, 2 H), 3.21 (s, 3 H) |
| 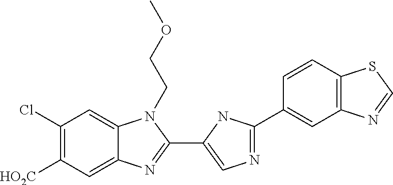 [SMILES: COCCn1c(-c2cnc(-c3ccc4scnc4c3)[nH]2)nc2cc(C(=O)O)c(Cl)cc21] | 82 | 454.1 | ¹H NMR (400 MHz, DMSO-d₆) ™ ppm 9.49 (s, 1 H), 8.76 (s, 1 H), 8.33 (d, J = 8.6 Hz, 1 H), 8.25 (s, 1 H), 8.21 (d, J = 8.6 Hz, 1 H), 8.04 (s, 1 H), 7.90 (s, 1 H), 5.09 (t, J = 4.9 Hz, 2 H), 3.88 (t, J = 5.2 Hz, 2 H), 3.22 (s, 3 H) |
| 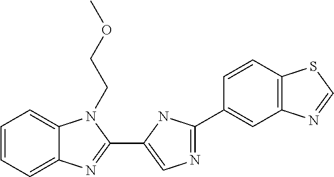 [SMILES: COCCn1c(-c2cnc(-c3ccc4scnc4c3)[nH]2)nc2ccccc21] | 83 | 376.2 | ¹H NMR (400 MHz, DMSO-d₆) ™ ppm 13.29 (br. s., 1 H), 9.48 (s, 1 H), 8.74 (s, 1 H), 8.31 (d, J = 8.6 Hz, 1 H), 8.19 (d, J = 8.3 Hz, 1 H), 8.08 (s, 1 H), 7.59 (t, J = 5.9 Hz, 2 H), 7.16-7.26 (m, 2 H), 5.06 (t, J = 5.4 Hz, 2 H), 3.88 (t, J = 5.4 Hz, 2 H), 3.24 (s, 3 H) |

### Example 84

### 5-{4-[1-[2-(methyloxy)ethyl]-4-(methylsulfonyl)-1H-benzimidazol-2-yl]-1H-imidazol-2-yl}-1,3-benzothiazole trifluoroacetate

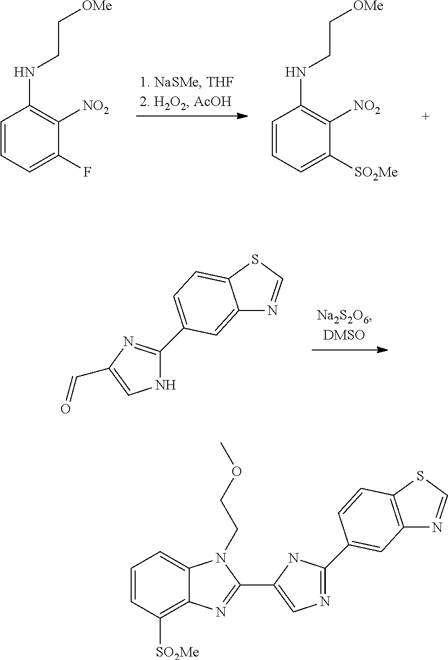 [SMILES: COCCNc1cccc(F)c1[N+](=O)[O-] ; COCCNc1cccc(S(C)(=O)=O)c1[N+](=O)[O-] ; O=Cc1c[nH]c(-c2ccc3scnc3c2)n1 ; COCCn1c(-c2cnc(-c3ccc4scnc4c3)[nH]2)nc2c(S(C)(=O)=O)cccc21]

Step 1. N-[2-(methyloxy)ethyl]-3-(methylsulfonyl)-2-nitroaniline: To a solution of (3-fluoro-2-nitrophenyl)[2-(methyloxy)ethyl]amine (75 mg, 0.350 mmol) in Tetrahydrofuran (THF) (923 μl) was added sodium thiomethoxide (29.5 mg, 0.420 mmol) at rt. After 3 hr, reaction had progressed, but still 17% stating material remained. Added more NaSMe (10 mg) and stirred for 1 hr. Reaction was complete by LCMS. Removed solvent in vacuo. Dissolved bright orange residue in Acetic Acid (923 μl). Added hydrogen peroxide (215 μl, 2.101 mmol) (30% aq solution, 9.79M). This was stirred at 80° C. overnight. LCMS showed significant product peak. Removed all volatiles and placed on high vac overnight. Purified via Biotage FCC (10 g SNAP column, 0-35% EtOAc/hex) yielding N-[2-(methyloxy)ethyl]-3-(methylsulfonyl)-2-nitroaniline (71.3 mg, 0.237 mmol, 67.6% yield) as a bright orange oil. MS (m/z): 215.0 (M+H)⁺.

Step 2. 5-{4-[1-[2-(methyloxy)ethyl]-4-(methylsulfonyl)-1H-benzimidazol-2-yl]-1H-imidazol-2-yl}-1,3-benzothiazole: A suspension of Sodium Dithionite (116 mg, 0.667 mmol) in Dimethyl Sulfoxide (DMSO) (556 μl) was heated to 80° C. for 5 min. at which time, a solution of 5-{4-[1-[2-(methyloxy)ethyl]-4-(methylsulfonyl)-1H-benzimidazol-2-yl]-1H-imidazol-2-yl}-1,3-benzothiazole (16.2 mg, 0.029 mmol, 12.83% yield) was added in Methanol (556 μl) followed by 2-(1,3-benzothiazol-5-yl)-1H-imidazole-4-carbaldehyde (51.0 mg, 0.222 mmol). This was stirred overnight at 80° C. LCMS showed mostly product. The crude reaction mixture was filtered through an acrodisc and purified on a Mass directed Agilent HPLC system, on a Waters Sunfire 30×150 mm column with a gradient of 17-51% acetonitrile/water (0.1% TFA) at a flow rate of 50 mL/min. The purified product was isolated as the TFA salt of 5-{4-[1-[2-(methyloxy)ethyl]-4-(methylsulfonyl)-1H-benzimidazol-2-yl]-1H-imidazol-2-yl}-1,3-benzothiazole (16.2 mg, 0.029 mmol, 12.83% yield). ¹H NMR (400 MHz, DMSO-d₆) ^{TM} ppm 9.49 (s, 1H), 8.77 (d, J=1.3 Hz, 1H), 8.31 (s, 1H), 8.19-8.27 (m, 2H), 8.00 (d, J=7.5 Hz, 1H), 7.72 (d, J=6.8 Hz, 1H), 7.42 (t, J=7.9 Hz, 1H), 5.16 (t, J=5.0 Hz, 2H), 3.91 (t, J=5.3 Hz, 2H), 3.58 (s, 3 H), 3.23 (s, 3H); MS (m/z): 454.1 (M+H)⁺.

The following compound was prepared with a procedure analogous to that described in Example 84.

| Structure | Example | MS (m/z) | ¹H NMR/Notes |
|---|---|---|---|
| 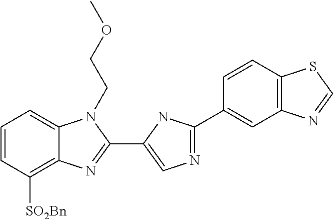 | 85 | 530.2 | ¹H NMR (400 MHz, DMSO-d₆) ™ ppm 9.50 (s, 1 H), 8.80 (s, 1 H), 8.30-8.39 (m, 2 H), 8.21-8.28 (m, 1 H), 7.99 (d, J = 8.0 Hz, 1 H), 7.50 (d, J = 7.8 Hz, 1 H), 7.32 (t, J = 7.7 Hz, 1 H), 7.24 (d, J = 6.8 Hz, 3 H), 7.15 (d, J = 7.3 Hz, 2 H), 5.19 (s, 4 H), 3.94 (t, J = 4.9 Hz, 2 H), 3.23 (s, 3 H) Step 1: Benzyl mercaptan Na2CO3 (1 equiv each) were used instead of NaSMe. |

### Example 86

### 5-{4-[1-[2-(methyloxy)ethyl]-4-(methylsulfonyl)-1H-benzimidazol-2-yl]-1H-imidazol-2-yl}-1,3-benzothiazole trifluoroacetate

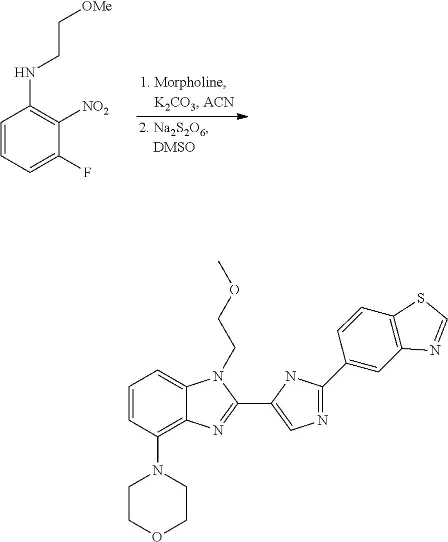 [SMILES: COCCNc1cccc(F)c1[N+](=O)[O-] ; COCCn1c(-c2cnc(-c3ccc4scnc4c3)[nH]2)nc2c(N3CCOCC3)cccc21]

Part 1. To a solution of (3-fluoro-2-nitrophenyl)[2-(methyloxy)ethyl]amine (50 mg, 0.233 mmol) in Acetonitrile (572 μl) was added potassium carbonate (64.5 mg, 0.467 mmol) and morpholine (22.37 μl, 0.257 mmol). This was heated to 80° C. for 90 min; LCMS (−90 m) showed complete conversion to the morpholine adduct.

Part 2. In another reaction vessel, Sodium dithionite (122 mg, 0.700 mmol) was added to Dimethyl Sulfoxide (DMSO) (572 μl) and this was heated to 80° C. for 5 min. At this time, the above reaction mixture was filtered into the DMSO and 2-(1,3-benzothiazol-5-yl)-1H-imidazole-4-carbaldehyde (53.5 mg, 0.233 mmol) was added immediately thereafter, rinsing with a small amt of ACN. Stirred reaction at 80° C. overnight. LCMS showed a major product peak. Cooled to rt and filtered through acrodisc and purified on a Mass directed Agilent HPLC system, on a Waters Sunfire 30×150 mm column with a gradient of 17-51% acetonitrile/water (0.1% TFA) at a flow rate of 50 mL/min to provide the product as the TFA salt of 5-{4-[1-[2-(methyloxy)ethyl]-4-(4-morpholinyl)-1H-benzimidazol-2-yl]-1H-imidazol-2-yl}-1,3-benzothiazole (33.4 mg, 0.058 mmol, 24.90° A yield). ¹H NMR (400 MHz, DMSO-d₆) ^{TM} ppm 9.51 (s, 1H), 8.78 (s, 1H), 8.35 (d, J=8.3 Hz, 1H), 8.27 (none, 1H), 8.22 (dd, J=8.3, 1.5 Hz, 1H), 7.24-7.47 (m, 2H), 6.84 (br. s., 1H), 5.03 (br. s., 2H), 3.88 (d, J=4.3 Hz, 6H), 3.32-3.43 (m, 4H), 3.23 (s, 3H); MS (m/z): 461.1 (M+H)⁺.

### Example 87

### 2-[2-(1,3-benzothiazol-5-yl)-1H-imidazol-4-yl]-1-[2-(methyloxy)ethyl]-4-(4-morpholinyl)-1H-imidazo[4,5-c]pyridine trifluoroacetate

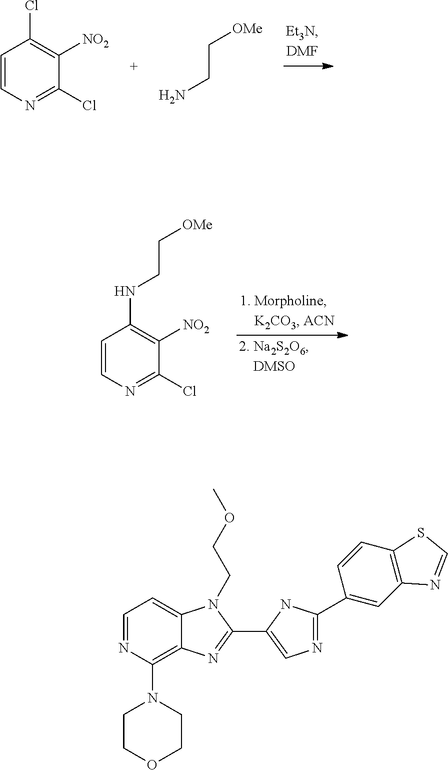 [SMILES: O=[N+]([O-])c1c(Cl)ccnc1Cl ; COCCN ; COCCNc1ccnc(Cl)c1[N+](=O)[O-] ; COCCn1c(-c2cnc(-c3ccc4scnc4c3)[nH]2)nc2c(N3CCOCC3)nccc21]

Step 1. 2-chloro-N-[2-(methyloxy)ethyl]-3-nitro-4-pyridinamine: To a solution of 2,4-dichloro-3-nitropyridine (1 g, 5.18 mmol) and triethylamine (0.780 ml, 5.60 mmol) in N,N-Dimethylformamide (DMF) (2.471 ml) at 0° C. was added a solution of 2-methoxyethlyamine (0.393 g, 5.23 mmol) in N,N-Dimethylformamide (DMF) (0.675 ml). Removed from ice bath and stirred at rt for 3 hr. After 1 hr of stirring, LCMS showed 50% conversion. Added triethylamine (0.780 ml, 5.60 mmol) and a thick slurry immediately formed. After 2 additional hour of stirring, LCMS showed mostly desired product. Quenched with water and diluted with EtOAc. Separated and extracted twice more with EtOAc. Washed combined organics with water twice, then with brine, dried on MgSO4, filtered and concentrated. Triturated resulting residue with Et₂O and filtered resulting in a bright yellow solid. Evaporated filtrate and further triturated residue with MTBE, filtering again and combining yellow residue with the first batch to provide 2-chloro-N-[2-(methyloxy)ethyl]-3-nitro-4-pyridinamine (667 mg, 2.88 mmol, 55.6% yield). MS (m/z): 232.0 (M+H)⁺.

Step 2. 2-[2-(1,3-benzothiazol-5-yl)-1H-imidazol-4-yl]-1-[2-(methyloxy)ethyl]-4-(4-morpholinyl)-1H-imidazo[4,5-c]pyridine:

Part 1: To a solution of 2-chloro-N-[2-(methyloxy)ethyl]-3-nitro-4-pyridinamine (50 mg, 0.216 mmol) in Acetonitrile (530 μl) was added potassium carbonate (31.3 mg, 0.227 mmol) followed by morpholine (19.75 μl, 0.227 mmol). This was stirred vigorously at rt for 2 hr, at which time, LCMS showed complete reaction.

Part 2: In another reaction vessel, Sodium dithionite (133 mg, 0.648 mmol) was added to Dimethyl Sulfoxide (DMSO) (530 μl) and this was heated to 80° C. for 5 min. At this time, the above reaction mixture was filtered into the DMSO and 2-(1,3-benzothiazol-5-yl)-1H-imidazole-4-carbaldehyde (49.5 mg, 0.216 mmol) was added immediately thereafter, rinsing with a small amt of ACN. Stirred reaction overnight. LCMS showed a major product peak. The crude reaction mixture was filtered through an acrodisc and was purified on a Mass directed Agilent HPLC system, on a Waters Sunfire 30×150 mm column with a gradient of 26-60% acetonitrile/water (0.1% TFA) to provide the product as a TFA salt of 2-[2-(1,3-benzothiazol-5-yl)-1H-imidazol-4-yl]-1-[2-(methyloxy)ethyl]-4-(4-morpholinyl)-1H-imidazo[4,5-c]pyridine (12.9 mg, 0.022 mmol, 10.38% yield). ¹H NMR (400 MHz, DMSO-d₆) ^{TM} ppm 9.49 (s, 1H), 8.75 (s, 1H), 8.32 (d, J=8.5 Hz, 1H), 8.17-8.23 (m, 2H), 7.78 (d, J=6.8 Hz, 1H), 7.36-7.45 (m, 1H), 5.13 (br. s., 2H), 4.25 (br. s., 4 H), 3.84-3.95 (m, 4H), 3.76-3.84 (m, 2H), 3.19 (s, 3H); MS (m/z): 462.2 (M+H)⁺.

The following compounds were prepared with a procedure analogous to that described in Example 87.

| Structure | Example | MS (m/z) | ¹H NMR/Notes |
|---|---|---|---|
| 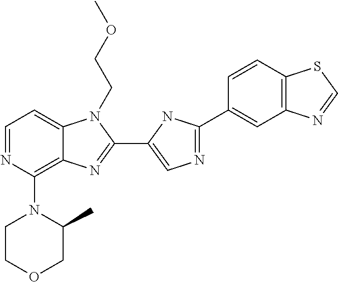 [SMILES: COCCn1c(-c2cnc(-c3ccc4scnc4c3)[nH]2)nc2c(N3CCOC[C@@H]3C)nccc21] | 88 | 476.2 | ¹H NMR (400 MHz, DMSO-d₆) ™ ppm 9.49 (s, 1 H), 8.75 (s, 1 H), 8.32 (d, J = 8.3 Hz, 1 H), 8.19 (d, J = 8.5 Hz, 1 H), 8.17 (s, 1 H), 7.76 (d, J = 6.8 Hz, 1 H), 7.39 (d, J = 6.8 Hz, 1 H), 5.22-5.38 (m, 1 H), 5.12 (m, J = 5.9 Hz, 1 H), 4.74-4.94 (m, 1 H), 4.06 (br. s., 1 H), 3.88 (t, J = 5.1 Hz, 2 H), 3.84 (s, 2 H), 3.68 (m, J = 6.1 Hz, 3 H), 3.20 (s, 3 H), 1.43 (d, J = 6.8 Hz, 3 H) |
| 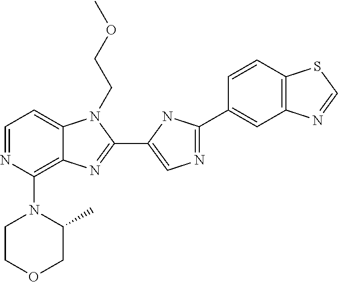 [SMILES: COCCn1c(-c2cnc(-c3ccc4scnc4c3)[nH]2)nc2c(N3CCOC[C@H]3C)nccc21] | 89 | 476.2 | ¹H NMR (400 MHz, DMSO-d₆) ™ ppm 9.49 (s, 1 H), 8.75 (s, 1 H), 8.32 (d, J = 8.5 Hz, 1 H), 8.20 (d, J = 8.5 Hz, 1 H), 8.17 (s, 1 H), 7.76 (d, J = 6.8 Hz, 1 H), 7.40 (d, J = 6.8 Hz, 1 H), 5.27 (br. s., 1 H), 5.13 (br. s., 2 H), 4.83 (br. s., 1 H), 4.07 (d, J = 7.8 Hz, 1 H), 3.89 (t, J = 5.1 Hz, 2 H), 3.84 (s, 2 H), 3.61- 3.75 (m, 2 H), 3.20 (s, 3 H), 1.43 (d, J = 6.8 Hz, 3 H) |
| 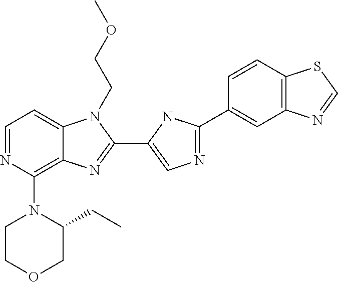 [SMILES: CC[C@@H]1COCCN1c1nccc2c1nc(-c1cnc(-c3ccc4scnc4c3)[nH]1)n2CCOC] | 90 | 490.3 | ¹H NMR (400 MHz, DMSO-d₆) ™ ppm 9.44- 9.53 (m, 1 H), 8.75 (s, 1 H), 8.32 (d, J = 8.3 Hz, 1 H), 8.19 (d, J = 8.3 Hz, 1 H), 8.13 (s, 1 H), 7.75 (d, J = 6.8 Hz, 1 H), 7.38 (d, J = 7.1 Hz, 1 H), 5.25 (br. s., 1 H), 5.03-5.21 (m, 2 H), 4.61 (br. s., 1 H), 4.05 (br. s., 1 H), 3.93-4.00 (m, 1 H), 3.89 (t, J = 5.0 Hz, 2 H), 3.79 (dd, J = 11.8, 2.6 Hz, 1 H), 3.59- 3.74 (m, 2 H), 3.21 (s, 3 H), 1.78-2.03 (m, 2 H), 0.94 (t, J = 7.3 Hz, 3 H) |
| 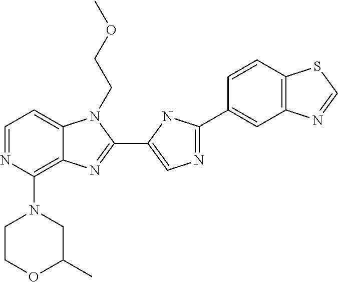 [SMILES: COCCn1c(-c2cnc(-c3ccc4scnc4c3)[nH]2)nc2c(N3CCOC(C)C3)nccc21] | 91 | 476.2 | ¹H NMR (400 MHz, DMSO-d₆) ™ ppm 9.49 (s, 1 H), 8.75 (s, 1 H), 8.32 (d, J = 8.3 Hz, 1 H), 8.11-8.25 (m, 2 H), 7.78 (d, J = 6.8 Hz, 1 H), 7.36 (br. s., 1 H), 5.12 (br. s., 3 H), 4.89 (br. s., 1 H), 4.02 (br. s., 1 H), 3.88 (t, J = 4.9 Hz, 2 H), 3.68-3.83 (m, 3 H), 3.20 (s, 3 H), 3.04-3.14 (m, 1 H), 1.23 (d, J = 6.3 Hz, 3 H) |
| 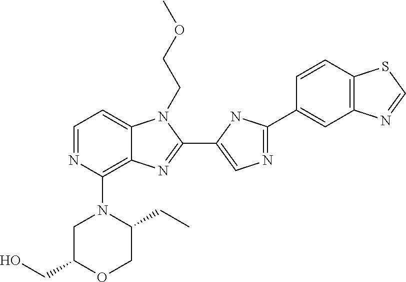 [SMILES: CC[C@@H]1CO[C@H](CO)CN1c1nccc2c1nc(-c1cnc(-c3ccc4scnc4c3)[nH]1)n2CCOC] | 92 | 520.3 | ¹H NMR (400 MHz, DMSO-d₆) ™ ppm 9.49 (s, 1 H), 8.75 (s, 1 H), 8.32 (d, J = 8.3 Hz, 1 H), 8.19 (d, J = 8.3 Hz, 1 H), 8.12 (s, 1 H), 7.75 (d, J = 7.1 Hz, 1 H), 7.38 (d, J = 6.6 Hz, 1 H), 5.13 (d, J = 4.5 Hz, 3 H), 4.01 (d, J = 11.9 Hz, 2 H), 3.89 (t, J = 5.2 Hz, 4 H), 3.84 (dd, J = 12.1, 2.8 Hz, 2 H), 3.53-3.64 (m, 2 H), 3.31-3.41 (m, 1 H), 3.21 (s, 3 H), 2.84-3.08 (m, 1 H), 1.79-2.04 (m, 2 H), 0.93 (t, J = 7.3 Hz, 3 H) |
| 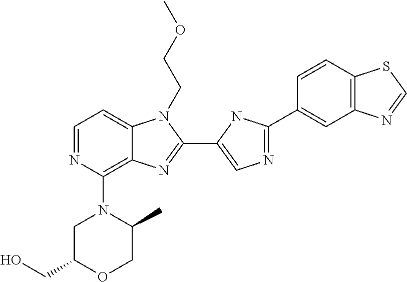 [SMILES: COCCn1c(-c2cnc(-c3ccc4scnc4c3)[nH]2)nc2c(N3C[C@@H](CO)OC[C@@H]3C)nccc21] | 93 | 506.2 | ¹H NMR (500 MHz, DMSO-d₆) ™ ppm 9.48 (s, 1 H), 8.75 (s, 1 H), 8.29-8.37 (m, 2 H), 8.25 (s, 2 H), 7.73 (d, J = 7.1 Hz, 1 H), 7.37 (d, J = 6.8 Hz, 1 H), 5.11 (br. s., 2 H), 4.97- 5.06 (m, 1 H), 4.91 (br. s., 1 H), 4.22 (br. s., 1 H), 4.08-4.17 (m, 1 H), 3.96-4.07 (m, 2 H), 3.89 (d, J = 4.9 Hz, 2 H), 3.55-3.67 (m, 2 H), 3.21 (s, 3 H), 1.43 (d, J = 6.6 Hz, 3 H) |
| 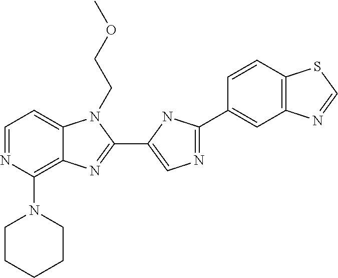 [SMILES: COCCn1c(-c2cnc(-c3ccc4scnc4c3)[nH]2)nc2c(N3CCCCC3)nccc21] | 94 | 460.2 | ¹H NMR (400 MHz, DMSO-d₆) ™ ppm 12.57 (br. s., 1 H), 9.49 (s, 1 H), 8.75 (d, J = 1.3 Hz, 1 H), 8.32 (d, J = 8.5 Hz, 1 H), 8.19 (dd, J = 8.3, 1.5 Hz, 1 H), 8.16 (s, 1 H), 7.71 (d, J = 6.8 Hz, 1 H), 7.34 (d, J = 7.0 Hz, 1 H), 5.11 (t, J = 4.8 Hz, 2 H), 4.24 (br. s., 4 H), 3.88 (t, J = 5.0 Hz, 2 H), 3.20 (s, 3 H), 1.75 (br. s., 6 H) |
| 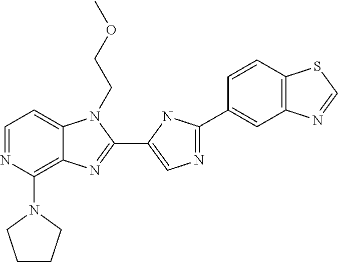 [SMILES: COCCn1c(-c2cnc(-c3ccc4scnc4c3)[nH]2)nc2c(N3CCCC3)nccc21] | 95 | 446.2 | ¹H NMR (500 MHz, DMSO-d₆) ™ ppm 12.28 (d, J = 4.6 Hz, 1 H), 9.48 (s, 1 H), 8.74 (s, 1 H), 8.31 (d, J = 8.3 Hz, 1 H), 8.19 (d, J = 8.3 Hz, 1 H), 8.13 (s, 1 H), 7.66 (br. s., 1 H), 7.26 (d, J = 7.1 Hz, 1 H), 5.11 (br. s., 2 H), 3.93-4.25 (m, 4 H), 3.87 (t, J = 4.8 Hz, 2 H), 3.19 (s, 3 H), 2.08 (t, J = 6.2 Hz, 4 H) |
| 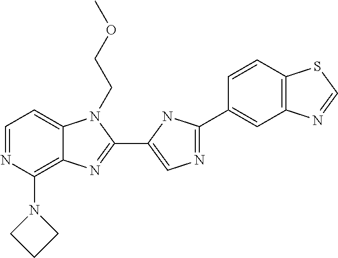 [SMILES: COCCn1c(-c2cnc(-c3ccc4scnc4c3)[nH]2)nc2c(N3CCC3)nccc21] | 96 | 432.2 | ¹H NMR (400 MHz, DMSO-d₆) ™ ppm 12.71 (br. s., 1 H), 9.49 (s, 1 H), 8.74 (s, 1 H), 8.31 (d, J = 8.3 Hz, 1 H), 8.18 (dd, J = 8.3, 1.5 Hz, 1 H), 8.08 (s, 1 H), 7.68 (d, J = 7.0 Hz, 1 H), 7.24 (d, J = 7.0 Hz, 1 H), 5.09 (t, J = 3.9 Hz, 2 H), 4.63 (br. s., 4 H), 3.86 (t, J = 5.0 Hz, 2 H), 3.19 (s, 3 H), 2.55 (m, J = 8.5 Hz, 2 H) |
| 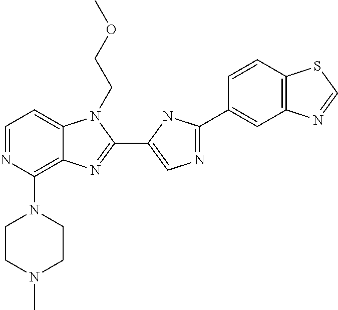 [SMILES: COCCn1c(-c2cnc(-c3ccc4scnc4c3)[nH]2)nc2c(N3CCN(C)CC3)nccc21] | 97 | 475.2 | ¹H NMR (400 MHz, DMSO-d₆) ™ ppm 9.50 (s, 1 H), 8.75 (d, J = 1.3 Hz, 1 H), 8.33 (d, J = 8.5 Hz, 1 H), 8.14-8.22 (m, 2 H), 7.90 (d, J = 6.3 Hz, 1 H), 7.37 (br. s., 1 H), 5.10 (br. s., 2 H), 3.88 (t, J = 5.1 Hz, 4 H), 3.62 (br. s., 4 H), 3.20 (s, 3 H), 2.89 (s, 3 H) |
| 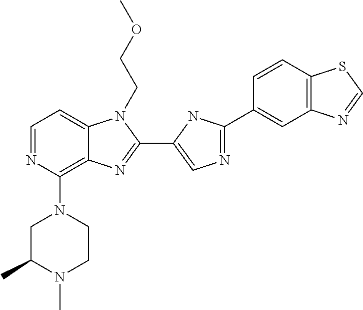 [SMILES: COCCn1c(-c2cnc(-c3ccc4scnc4c3)[nH]2)nc2c(N3CCN(C)[C@@H](C)C3)nccc21] | 98 | 489.5 | ¹H NMR (400 MHz, DMSO-d₆) ™ ppm 13.28 (br. s., 1 H), 9.48 (s, 1 H), 8.74 (s, 1 H), 8.30 (d, J = 8.3 Hz, 1 H), 8.14-8.25 (m, 1 H), 8.00 (s, 1 H), 7.80 (d, J = 5.8 Hz, 1 H), 6.96 (d, J = 5.6 Hz, 1 H), 5.17 (br. s., 1 H), 4.89-5.08 (m, 3 H), 3.74-3.94 (m, 2 H), 3.21 (s, 3 H), 3.10-3.22 (m, 2 H), 2.71- 3.04 (m, 4 H), 2.24 (s, 3 H), 2.00-2.17 (m, 1 H), 1.10 (d, J = 5.6 Hz, 3 H) |
| 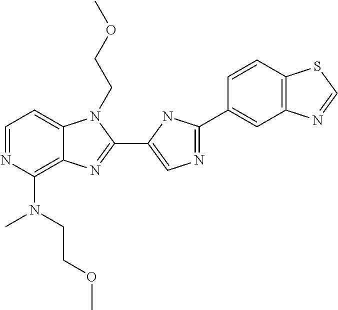 [SMILES: COCCN(C)c1nccc2c1nc(-c1cnc(-c3ccc4scnc4c3)[nH]1)n2CCOC] | 99 | 464.2 | ¹H NMR (400 MHz, MeOH-d₄) ™ ppm 9.35 (s, 1 H), 8.68 (s, 1 H), 8.19-8.24 (m, 1 H), 8.16 (dd, J = 8.6, 1.5 Hz, 1 H), 8.01 (s, 1 H), 7.62 (d, J = 7.1 Hz, 1 H), 7.30 (d, J = 7.1 Hz, 1 H), 5.11 (t, J = 4.8 Hz, 2 H), 4.58 (t, J = 4.9 Hz, 2 H), 3.98 (t, J = 4.9 Hz, 2 H), 3.84 (t, J = 5.1 Hz, 2 H), 3.57 (s, 3 H), 3.40 (s, 3 H), 3.29 (br. s., 3 H) |
| 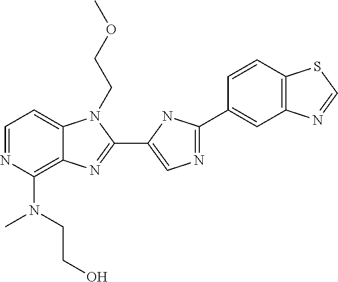 [SMILES: COCCn1c(-c2cnc(-c3ccc4scnc4c3)[nH]2)nc2c(N(C)CCO)nccc21] | 100 | 450.2 | ¹H NMR (400 MHz, DMSO-d₆) ™ ppm 13.46 (br. s., 1 H), 12.33 (br. s., 1 H), 9.49 (s, 1 H), 8.75 (d, J = 1.3 Hz, 1 H), 8.32 (d, J = 8.3 Hz, 1 H), 8.19 (dd, J = 8.3, 1.5 Hz, 1 H), 8.15 (s, 1 H), 7.70 (d, J = 7.5 Hz, 1 H), 7.32 (d, J = 7.0 Hz, 1 H), 5.11 (t, J = 4.5 Hz, 2 H), 4.36 (br. s., 2 H), 3.88 (t, J = 4.9 Hz, 2 H), 3.80 (t, J = 5.3 Hz, 2 H), 3.65 (s, 3 H), 3.20 (s, 3 H) |
| 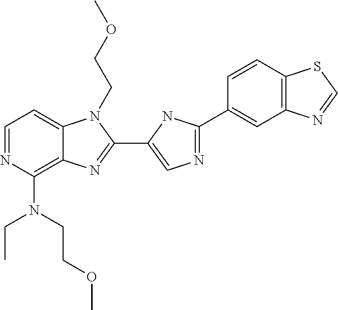 [SMILES: CCN(CCOC)c1nccc2c1nc(-c1cnc(-c3ccc4scnc4c3)[nH]1)n2CCOC] | 101 | 478.3 | ¹H NMR (500 MHz, DMSO-d₆) ™ ppm 12.27 (br. s., 1 H), 9.49 (s, 1 H), 8.75 (s, 1 H), 8.32 (d, J = 8.5 Hz, 1 H), 8.19 (dd, J = 8.3, 1.2 Hz, 1 H), 8.08 (s, 1 H), 7.69 (d, J = 6.1 Hz, 1 H), 7.31 (d, J = 7.1 Hz, 1 H), 5.10 (t, J = 4.0 Hz, 2 H), 4.30 (t, J = 5.0 Hz, 2 H), 4.09 (q, J = 6.6 Hz, 2 H), 3.89 (t, J = 4.9 Hz, 2 H), 3.74 (t, J = 5.1 Hz, 2 H), 3.21 (s, 6 H), 1.31 (t, J = 7.0 Hz, 3 H) |

### Example 102

### 2-[2-(1,3-benzothiazol-5-yl)-1H-imidazol-4-yl]-6-methyl-1-[2-(methyloxy)ethyl]-4-(4-morpholinyl)-1H-imidazo[4,5-c]pyridine

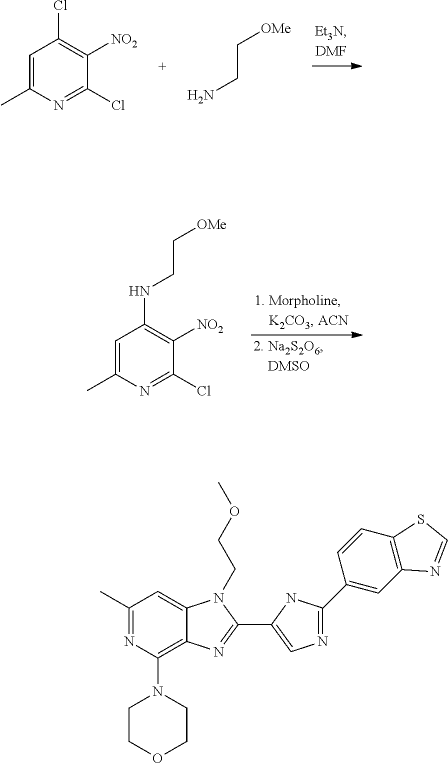 [SMILES: Cc1cc(Cl)c([N+](=O)[O-])c(Cl)n1 ; COCCN ; COCCNc1cc(C)nc(Cl)c1[N+](=O)[O-] ; COCCn1c(-c2cnc(-c3ccc4scnc4c3)[nH]2)nc2c(N3CCOCC3)nc(C)cc21]

### Step 1. 2-chloro-6-methyl-N-[2-(methyloxy)ethyl]-3-nitro-4-pyridinamine

To a solution of 2,4-dichloro-6-methyl-3-nitropyridine (1 g, 4.83 mmol) and triethylamine (0.741 ml, 5.31 mmol) in N,N-Dimethylformamide (DMF) (1.959 ml) at 0° C. was added a solution of 2-methoxyethlyamine (0.424 ml, 4.88 mmol) in N,N-Dimethylformamide (DMF) (0.535 ml). Removed from ice bath and stirred at rt overnight. LCMS showed mainly desired product along with a small amount of the undesired regioisomer as well as the bis addition product. Quenched with water and diluted with Et2O. Separated and extracted twice more with Et2O. Washed combined organics with water twice, then with brine, dried on MgSO4, filtered and concentrated. Purified via Biotage FCC (0-20% EtOAc/hex) Desired and bis-addition product co-eluted. Combined all product-containing fractions and concentrated resulting in a bright yellow solid. Suspended in hexanes, sonicating to break up large particles. Sonicated for 20 min. Filtered and collected bright yellow solid that was pure desired product: 2-chloro-6-methyl-N-[2-(methyloxy)ethyl]-3-nitro-4-pyridinamine (466 mg, 1.897 mmol, 39.3% yield). MS (m/z): 246.1 (M+H)⁺.

### Step 2. 2-[2-(1,3-benzothiazol-5-yl)-1H-imidazol-4-yl]-1-[2-(methyloxy)ethyl]-4-(4-morpholinyl)-1H-imidazo[4,5-c]pyridine

Part 1. To a solution of 2-chloro-6-methyl-N-[2-(methyloxy)ethyl]-3-nitro-4-pyridinamine (80 mg, 0.327 mmol) in Acetonitrile (803 μl) was added potassium carbonate (90 mg, 0.654 mmol) and morpholine (29.9 μl, 0.343 mmol). This was heated to 80° C. for 30 min; LCMS (−30 m) showed complete conversion to the morpholine adduct.

Part 2. In another reaction vessel, Sodium dithionite (171 mg, 0.981 mmol) was added to Dimethyl Sulfoxide (DMSO) (803 μl) and this was heated to 80° C. for 5 min. At this time, the above reaction mixture was filtered into the DMSO and 2-(1,3-benzothiazol-5-yl)-1H-imidazole-4-carbaldehyde (75 mg, 0.327 mmol) was added immediately thereafter, rinsing with a small amt of ACN. Stirred reaction at 80° C. overnight. Cooled to rt and filtered through acrodisc. The crude reaction mixture was filtered through an acrodisc and purified via Gilson (10-75% ACN (0.1% TFA)/H20 (0.1% TFA). Combined product fractions and neutralized with MP-carbonate. Filtered and removed solvent. Triturated resultant solid with Et₂O, filtered, providing 2-[2-(1,3-benzothiazol-5-yl)-1H-imidazol-4-yl]-6-methyl-1-[2-(methyloxy)ethyl]-4-(4-morpholinyl)-1H-imidazo[4,5-c]pyridine (22.5 mg, 0.047 mmol, 14.46% yield) as a brown solid. ¹H NMR (400 MHz, DMSO-d₆) ^{TM} ppm 13.27 (br. s., 1H), 9.47 (s, 1H), 8.73 (s, 1H), 8.30 (d, J=8.3 Hz, 1H), 8.13-8.23 (m, 1H), 7.98 (d, J=2.3 Hz, 1H), 6.84 (s, 1H), 4.92 (t, J=5.6 Hz, 2H), 4.06 (t, J=4.5 Hz, 4H), 3.81 (t, J=5.7 Hz, 2H), 3.76 (t, J=5.1 Hz, 4H), 3.21 (s, 3H), 2.40 (s, 3H); MS (m/z): 462.2 (M+H)⁺.

### Example 103

### 5-{4-[1-[2-(methyloxy)ethyl]-5-(4-morpholinylsulfonyl)-1H-benzimidazol-2-yl]-1H-imidazol-2-yl}-1,3-benzothiazole

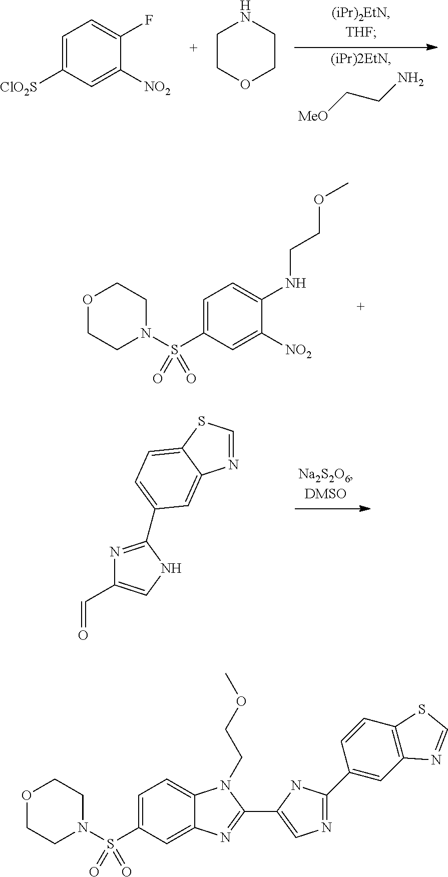

Step 1. N-[2-(methyloxy)ethyl]-4-(4-morpholinylsulfonyl)-2-nitroaniline[2-(methyloxy)ethyl][4-(4-morpholinylsulfonyl)-2-nitrophenyl]amine: Added morpholine (73.0 μl, 0.835 mmol) to 4-fluoro-3-nitrobenzenesulfonyl chloride (200 mg, 0.835 mmol) and 1 equiv. of DIEA in Tetrahydrofuran (THF) (2199 μl). Stirred at RT for two hours. LCMS showed product. Added [2-(methyloxy)ethyl]amine (72.6 μl, 0.835 mmol) and 2 equiv. of DIEA (874 μl, 5.008 mmol) (×2). Heated the reaction to 150° C. in the microwave. LCMS looked good; material was carried on without further purification to the next step. MS (m/z) 346.3 (M+H)⁺.

Step 2. 5-{4-[1-[2-(methyloxy)ethyl]-5-(4-morpholinylsulfonyl)-1H-benzimidazol-2-yl]-1H-imidazol-2-yl}-1,3-benzothiazole: Added 2-(1,3-benzothiazol-5-yl)-1H-imidazole-4-carbaldehyde (222 mg, 0.835 mmol) to a slurry of N-[2-(methyloxy)ethyl]-4-(4-morpholinylsulfonyl)-2-nitroaniline (288 mg, 0.835 mmol) and Sodium Hydrosulfite (436 mg, 2.505 mmol) in Ethanol (2783 μl) and heated to 80° C. overnight. LCMS showed reaction was complete. Concentrated the reaction onto Celite and purified via Gilson (10-65% ACN/H2O). Isolated the desired fractions, and stirred with MP-carbonate to freebase. Filtered and concentrated to give material that was still slightly impure. Triturated with a small amount of MTBE, filtered and dried on high vac to provide: 5-{4-[1-[2-(methyloxy)ethyl]-5-(4-morpholinylsulfonyl)-1H-benzimidazol-2-yl]-1H-imidazol-2-yl}-1,3-benzothiazole (50 mg, 0.095 mmol, 11.41% yield). ¹H NMR (400 MHz, DMSO-d₆) ^{TM} ppm 13.42 (br. s., 1H), 9.49 (s, 1H), 8.75 (d, J=1.3 Hz, 1H), 8.67-8.84 (m, 1H), 8.32 (d, J=8.3 Hz, 1H), 8.17-8.24 (m, 2H), 7.93 (d, J=1.5 Hz, 1H), 7.87 (d, J=8.6 Hz, 1H), 7.58 (dd, J=8.5, 1.6 Hz, 1H), 5.15 (t, J=5.2 Hz, 2H), 3.93 (t, J=5.3 Hz, 2H), 3.57-3.69 (m, 2 H), 3.24 (s, 4H), 2.89 (d, J=4.3 Hz, 4H); MS (m/z): 525.2 (M+H)⁺.

The following compounds were prepared with a procedure analogous to that described in Example 103.

| Structure | Example | MS (m/z) | ¹H NMR/Notes |
|---|---|---|---|
| 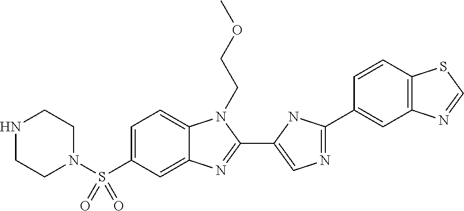 [SMILES: COCCn1c(-c2cnc(-c3ccc4scnc4c3)[nH]2)nc2cc(S(=O)(=O)N3CCNCC3)ccc21] | 104 | 524.2 | ¹H NMR (400 MHz, DMSO-d₆) ™ ppm 13.46 (bs, 1 H), 9.50 (s, 1 H), 8.76 (d, J = 1.3 Hz, 1 H), 8.50 (br. s., 1 H), 8.33 (d, J = 8.3 Hz, 1 H), 8.13-8.26 (m, 2 H), 7.99 (d, J = 1.8 Hz, 1 H), 7.93 (d, J = 8.5 Hz, 1 H), 7.64 (dd, J = 8.4, 1.6 Hz, 1 H), 5.15 (t, J = 4.9 Hz, 2 H), 3.94 (t, J = 5.3 Hz, 2 H), 3.25 (s, 3 H), 3.21 (br. s., 4 H), 3.12 (br. s., 4 H) |
| 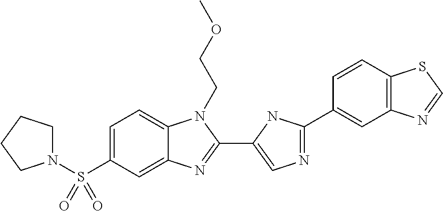 [SMILES: COCCn1c(-c2cnc(-c3ccc4scnc4c3)[nH]2)nc2cc(S(=O)(=O)N3CCCC3)ccc21] | 105 | 509.2 | ¹H NMR (400 MHz, DMSO-d₆) ™ ppm 13.69 (br. s., 1 H), 9.50 (s, 1 H), 8.77 (s, 1 H), 8.33 (d, J = 8.5 Hz, 1 H), 8.26 (s, 1 H), 8.21 (dd, J = 8.4, 1.4 Hz, 1 H), 8.00 (s, 1 H), 7.90 (d, J = 8.5 Hz, 1 H), 7.72 (d, J = 8.3 Hz, 1 H), 5.12 (t, J = 4.8 Hz, 2 H), 3.92 (t, J = 5.4 Hz, 2 H), 3.23 (s, 3 H), 3.12-3.21 (m, 4 H), 1.60-1.68 (m, 4 H) |
| 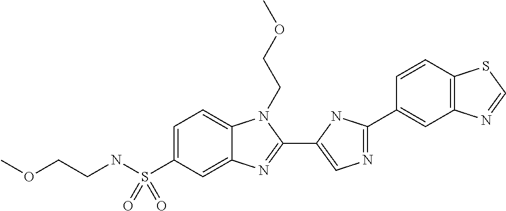 [SMILES: COCCNS(=O)(=O)c1ccc2c(c1)nc(-c1cnc(-c3ccc4scnc4c3)[nH]1)n2CCOC] | 106 | 513.2 | ¹H NMR (400 MHz, DMSO-d₆) ™ ppm 13.40 (br. s., 1 H), 9.49 (s, 1 H), 8.75 (s, 1 H), 8.32 (d, J = 8.3 Hz, 1 H), 8.20 (dd, J = 9.6, 1.3 Hz, 2 H), 7.99 (d, J = 1.5 Hz, 1 H), 7.80 (d, J = 8.6 Hz, 1 H), 7.66 (dd, J = 8.6, 1.5 Hz, 1 H), 7.61 (t, J = 5.9 Hz, 1 H), 5.13 (t, J = 5.3 Hz, 2 H), 3.91 (t, J = 5.3 Hz, 2 H), 3.31 (d, J = 5.8 Hz, 2 H), 3.22 (s, 3 H), 3.16 (s, 3 H), 2.90 (q, J = 5.8 Hz, 2 H) |
| 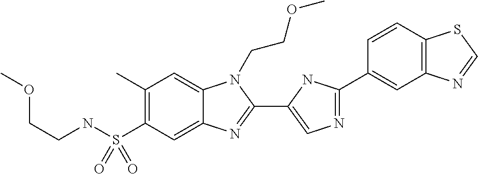 [SMILES: COCCNS(=O)(=O)c1cc2nc(-c3cnc(-c4ccc5scnc5c4)[nH]3)n(CCOC)c2cc1C] | 107 | 527.2 | 1H NMR (400 MHz, CHLOROFORM-d) d ppm 2.79 (s, 3 H) 3.03-3.16 (m, 2 H) 3.21- 3.28 (m, 3 H) 3.32-3.43 (m, 5 H) 3.99 (t, J = 5.05 Hz, 2 H) 4.92 (br. s., 2 H) 5.40- 5.61 (m, 1 H) 7.34 (s, 1 H) 7.85-8.14 (m, 3 H) 8.32 (s, 1 H) 8.67 (s, 1 H) 9.00-9.10 (m, 1 H) |
| 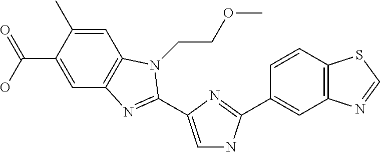 [SMILES: COCCn1c(-c2c[nH]c(-c3ccc4scnc4c3)n2)nc2cc(C(=O)O)c(C)cc21] | 108 | 434.2 | 1H NMR (400 MHz, DMSO-d6) d ppm 2.70 (s, 3 H) 3.24 (s, 3 H) 3.84-4.01 (m, 2 H) 4.95-5.19 (m, 2 H) 7.58-7.79 (m, 1 H) 8.10-8.17 (m, 1 H) 8.19-8.25 (m, 1 H) 8.27-8.31 (m, 1 H) 8.33-8.39 (m, 1 H) 8.73-8.85 (m, 1 H) 9.33-9.65 (m, 1 H) |
| 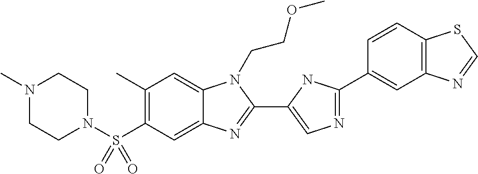 [SMILES: COCCn1c(-c2cnc(-c3ccc4scnc4c3)[nH]2)nc2cc(S(=O)(=O)N3CCN(C)CC3)c(C)cc21] | 109 | 551.9 | 1H NMR (400 MHz, METHANOL-d4) d ppm 2.52 (br. s., 3 H) 2.75 (br. s., 2 H) 2.96 (br. s., 2 H) 3.14-3.27 (m, 4 H) 3.30 (br. s., 3 H) 3.31 (br. s., 3 H) 3.86-4.06 (m, 2 H) 4.95-5.17 (m, 2 H) 7.57 (s, 1 H) 7.77- 7.87 (m, 1 H) 7.97-8.27 (m, 3 H) 8.58- 8.71 (m, 1 H) 9.31 (d, J = 2.02 Hz, 1 H) |
| 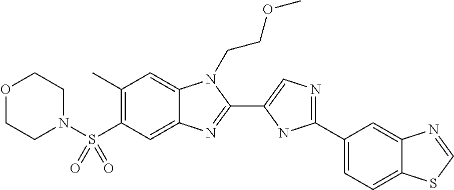 [SMILES: COCCn1c(-c2cnc(-c3ccc4scnc4c3)[nH]2)nc2cc(S(=O)(=O)N3CCOCC3)c(C)cc21] | 110 | 539.6 | 1H NMR (400 MHz, DMSO-d6) d ppm 2.73 (s, 3 H) 3.00-3.14 (m, 4 H) 3.25 (s, 3 H) 3.64 (br. s., 4 H) 3.89-3.99 (m, 2 H) 4.94- 5.19 (m, 2 H) 7.73-7.91 (m, 1 H) 8.06- 8.16 (m, 1 H) 8.18-8.27 (m, 1 H) 8.32- 8.49 (m, 2 H) 8.67-8.92 (m, 1 H) 9.51 (s, 1 H) |

### Example 111

### 2-[2-(1,3-benzothiazol-5-yl)-1H-imidazol-4-yl]-1-[2-(methyloxy)ethyl]-1H-benzimidazole-5-carboxylic acid

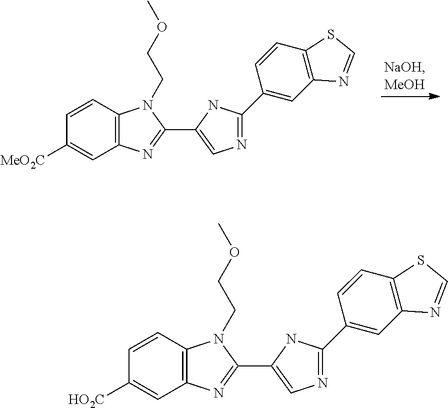 [SMILES: COCCn1c(-c2cnc(-c3ccc4scnc4c3)[nH]2)nc2cc(C(=O)OC)ccc21 ; COCCn1c(-c2cnc(-c3ccc4scnc4c3)[nH]2)nc2cc(C(=O)O)ccc21]

Heated methyl 2-[2-(1,3-benzothiazol-5-yl)-1H-imidazol-4-yl]-1-[2-(methyloxy)ethyl]-1H-benzimidazole-5-carboxylate (71 mg, 0.164 mmol) and NaOH (65.5 mg, 1.638 mmol) in Methanol (1.0 mL) to reflux overnight. Concentrated reaction onto Celite and ran a reverse phase C18 Isco column using a gradient of 10-100% CH3CN/water. Isolated the desired fractions to give 2-[2-(1,3-benzothiazol-5-yl)-1H-imidazol-4-yl]-1-[2-(methyloxy)ethyl]-1H-benzimidazole-5-carboxylic acid (6 mg, 0.012 mmol, 7.44% yield). ¹H NMR (400 MHz, DMSO-d₆) ^{TM} ppm 13.37 (br. s., 1H), 9.48 (s, 1H), 8.74 (d, J=1.3 Hz, 1H), 8.31 (d, J=8.6 Hz, 1H), 8.20 (dd, J=8.3, 1.5 Hz, 1H), 8.17 (s, 2H), 7.85 (dd, J=8.5, 1.4 Hz, 1H), 7.69 (d, J=8.6 Hz, 1H), 5.11 (t, J=5.4 Hz, 2H), 3.90 (t, J=5.4 Hz, 2H), 3.23 (s, 3H), 2.96 (br. s., 1H); MS (m/z): 420.4 (M+H)⁺.

### Example 112

### 5-(1-methyl-4-{6-methyl-1-[2-(methyloxy)ethyl]-1H-benzimidazol-2-yl}-1H-imidazol-2-yl)-1,3-benzothiazole trifluoroacetate

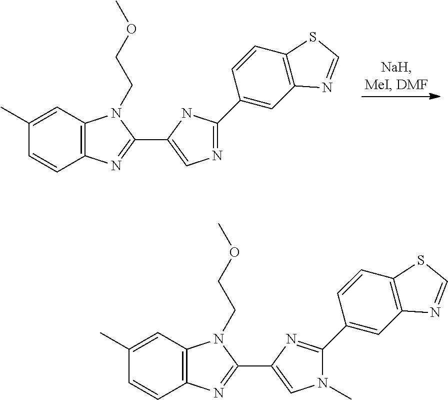 [SMILES: COCCn1c(-c2cnc(-c3ccc4scnc4c3)[nH]2)nc2ccc(C)cc21 ; COCCn1c(-c2cn(C)c(-c3ccc4scnc4c3)n2)nc2ccc(C)cc21]

Added sodium hydride (47.0 mg, 1.174 mmol) to 5-(4-{6-methyl-1-[2-(methyloxy)ethyl]-1H-benzimidazol-2-yl}-1H-imidazol-2-yl)-1,3-benzothiazole (200 mg, 0.470 mmol) in N,N-Dimethylformamide (DMF) (4663 μl) at 0° C. Stirred for ten minutes and added methyl iodide (32.3 μl, 0.517 mmol). Reaction was stirred overnight at room temperature. Poured the reaction onto water and extracted with EtOAc. Washed with water and dried with MgSO4 and concentrated onto Celite. Ran a reverse phase C18 Isco column using a gradient of 10-100% CH3CN/water. Isolated the desired fractions and concentrated. Product was still not clean, so it was taken up in CH3CN/MeOH. The crude reaction mixture was filtered through an acrodisc and was purified on a Mass directed Agilent HPLC system, on a Waters Sunfire 30×150 mm column with a gradient of 20-60% acetonitrile/water (0.1% TFA) to provide the product as a TFA salt of 5-(1-methyl-4-{6-methyl-1-[2-(methyloxy)ethyl]-1H-benzimidazol-2-yl}-1H-imidazol-2-yl)-1,3-benzothiazole (89 mg, 0.221 mmol, 47.0% yield). This structure was confirmed by NMR. ¹H NMR (400 MHz, DMSO-d₆) ^{TM} ppm 9.54 (s, 1H), 8.55 (d, J=1.3 Hz, 1H), 8.45 (s, 1H), 8.40 (d, J=8.5 Hz, 1H), 7.96 (dd, J=8.4, 1.6 Hz, 1H), 7.73 (s, 1H), 7.63 (d, J=8.3 Hz, 1H), 7.34 (d, J=8.3 Hz, 1H), 5.00 (t, J=5.0 Hz, 2H), 4.01 (s, 3H), 3.89 (t, J=5.1 Hz, 2H), 3.22 (s, 3H), 2.51 (br. s., 3H); MS (m/z): 404.2 (M+H)⁺.

### Example 113

### 2′-(1,3-benzothiazol-5-yl)-1-[2-(methyloxy)ethyl]-1H,1′H-2,4′-biimidazole

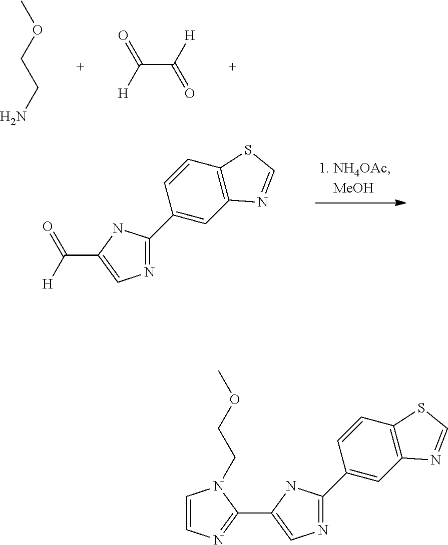

To a solution of 2-(1,3-benzothiazol-5-yl)-1H-imidazole-4-carbaldehyde (61.0 mg, 0.266 mmol) in Methanol (479 μl) was added 2-methoxyethylamine (23.15 μl, 0.266 mmol). This was stirred at rt for 5 min. Glyoxal (30.4 μl, 0.266 mmol) and Ammonium acetate (20.53 mg, 0.266 mmol) were then added and the reaction was heated to 80° C. (in a capped microwave vial). After 1.5 hr, removed reaction from heat. Purified crude reaction mixture with Gilson 10-60% ACN/H₂O (0.1% TFA). Combined product fractions and basified with MP-carbonate. Filtered and removed most of solvent. Removed remaining water on blowdown unit. Tool up in 2:1 ACN/H₂O and lyophilized resulting in 2′-(1,3-benzothiazol-5-yl)-1-[2-(methyloxy)ethyl]-1H,1′H-2,4′-biimidazole (19.5 mg, 0.060 mmol, 22.51% yield). ¹H NMR (400 MHz, MeOH-d₄) ^{TM} ppm 9.36 (s, 1H), 8.69 (s, 1H), 8.20-8.26 (m, 1H), 8.17 (dd, J=8.6, 1.3 Hz, 1H), 8.09 (s, 1H), 7.64 (d, J=2.0 Hz, 1H), 7.57 (d, J=2.0 Hz, 1H), 4.84 (br. s., 2H), 3.92 (t, J=4.8 Hz, 2H), 3.37 (s, 3H); MS (m/z): 326.2 (M+H)⁺.

The following compounds were prepared with a procedure analogous to that described in Example 113.

| Structure | Example | MS (m/z) | ¹H NMR/Notes |
|---|---|---|---|
| 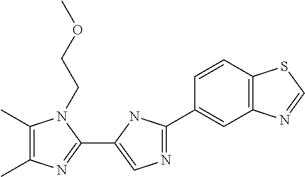 [SMILES: COCCn1c(-c2cnc(-c3ccc4scnc4c3)[nH]2)nc(C)c1C] | 114 | 354.1 | ¹H NMR (400 MHz, DMSO-d₆) ™ ppm 13.62 (br. s., 1 H), 9.50 (s, 1 H), 8.74 (s, 1 H), 8.34 (d, J = 8.3 Hz, 1 H), 8.18 (d, J = 1.3 Hz, 1 H), 8.15 (d, 1 H), 4.70 (t, J = 5.1 Hz, 2 H), 3.80 (t, J = 4.9 Hz, 2 H), 3.24 (s, 3 H), 2.31 (s, 3 H), 2.29 (s, 3 H) |
| 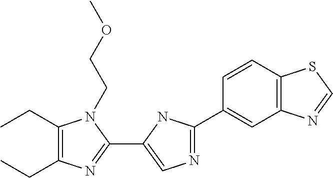 [SMILES: CCc1nc(-c2cnc(-c3ccc4scnc4c3)[nH]2)n(CCOC)c1CC] | 115 | 382.2 | ¹H NMR (400 MHz, MeOH-d₄) ™ 9.35 (s, 1 H), 8.68 (s, 1 H), 8.22 (d, J = 8.6 Hz, 1 H), 8.15 (m, J = 8.6 Hz, 1 H), 8.04 (s, 1 H), 4.78 (t, J = 4.9 Hz, 2 H), 3.87 (t, J = 5.1 Hz, 2 H), 3.31 (br. s., 3 H), 2.87 (q, J = 7.6 Hz, 2 H), 2.77 (q, J = 7.6 Hz, 2 H), 1.33 (t, J = 7.6 Hz, 3 H), 1.28 (t, J = 7.6 Hz, 3 H) |
| 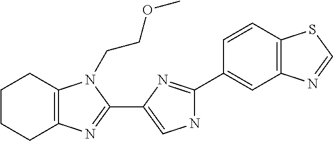 [SMILES: COCCn1c(-c2c[nH]c(-c3ccc4scnc4c3)n2)nc2c1CCCC2] | 116 | 380.2 | ¹H NMR (400 MHz, DMSO-d₆) ™ ppm 13.63 (br. s., 1 H), 9.51 (s, 1 H), 8.74 (d, J = 1.5 Hz, 1 H), 8.34 (d, J = 8.3 Hz, 1 H), 8.16 (dd, J = 8.5, 1.5 Hz, 2 H), 4.68 (t, J = 5.1 Hz, 2 H), 3.80 (t, J = 4.9 Hz, 3 H), 3.25 (s, 3 H), 2.69-2.77 (m, 2 H), 2.66 (br. s., 2 H), 1.83 (br. s., 4 H) |

^{a} LCMS Method: Agilent 1100 Series LC/MSD SL or VL using electrospray positive [ES+ve to give M+H⁺] equipped with a Sunfire C18 5.0 μm column (3.0 mm×50 mm, i.d.), eluting with 0.05% TFA in water (solvent A) and 0.05% TFA in acetonitrile (solvent B), using the following elution gradient: 10%-100% (solvent B) over 2.5 minutes and holding at 100% for 1.7 minutes at a flow rate of 1.0 mL/minutes.

^{b} LCMS Method: Agilent 1100 Series LC/MSD SL or VL using electrospray positive [ES+ve to give M+H⁺] equipped with a Sunfire C18 5.0 μm column (3.0 mm×50 mm, i.d.), eluting with 0.05% TFA in water (solvent A) and 0.05% TFA in acetonitrile (solvent B), using the following elution gradient 10%-100% (solvent B) over 10.0 minutes and holding at 100% for 1.7 minutes at a flow rate of 1.0 mL/minutes.

^{c} LCMS Method: On an Agilent 1200 Series LC/MSD VL using electrospray positive [ES+ve to give M+H⁺] equipped with a shim-pack XR-ODS 2.2 m column (3.0 mm×30 mm, 3.0 mm i.d.) eluting with 0.0375% TFA in water (solvent A) and 0.01875% TFA in acetonitrile (solvent B), using the following elution gradient 10-80% (solvent B) over 0.9 minutes and holding at 80% for 0.6 minutes at a flow rate of 1.2 mL/minutes.

### Pharmaceutical Compositions

### Example A

Tablets are prepared using conventional methods and are formulated as follows:

| Ingredient | Amount per tablet | |
|---|---|---|
| Compound of Example I | 5 | mg |
| Microcrystalline cellulose | 100 | mg |
| Lactose | 100 | mg |
| Sodium starch glycollate | 30 | mg |
| Magnesium stearate | 2 | mg |
| Total | 237 | mg |

### Example B

Capsules are prepared using conventional methods and are formulated as follows:

| Ingredient | Amount per tablet | |
|---|---|---|
| Compound of Example 3 | 15 | mg |
| Dried starch | 178 | mg |
| Magnesium stearate | 2 | mg |
| Total | 195 | mg |

### Biological In Vitro Assay:

A fluorescent polarization based binding assay was developed to quantitate interaction of novel test compounds at the ATP binding pocket of RIPK2, by competition with a fluorescently labeled ATP competitive ligand. Full length FLAG His tagged RIPK2 was purified from a Baculovirus expression system and was used at a final assay concentration of twice the KDapparent. A fluorescent labeled ligand (5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid, prepared as described below) was used at a final assay concentration of 5 nM. Both the enzyme and ligand were prepared in solutions in 50 mM HEPES pH7.5, 150 mM NaCl, 10 mM MgCl₂, 1 mM DTT, and 1 mM CHAPS. Test compounds were prepared in 100% DMSO and 100 mL was dispensed to individual wells of a multiwell plate. Next, 5 ul RIPK2 was added to the test compounds at twice the final assay concentration, and incubated at room temperature for 10 minutes. Following the incubation, 5 ul of the fluorescent labeled ligand solution, was added to each reaction, at twice the final assay concentration, and incubated at room temperature for at least 10 minutes. Finally, samples were read on an instrument capable of measuring fluorescent polarization. Test compound inhibition was expressed as percent (%) inhibition of internal assay controls.

For concentration response experiments, normalized data were fit and pIC₅₀s determined using conventional techniques. For example, the following four parameter logistic equation may be used: y=A+((B−C))/(1+(10^{x})/(10^{C})^{D}), where: y is the % activity (% inhibition) at a specified compound concentration; A is the minimum % activity; B is the maximum % activity; C=log₁₀(IC₅₀); D=Hill slope; x=log₁₀ (compound concentration [M]); and plC₅₀=(—C).

The pIC₅₀s are averaged to determine a mean value, for a minimum of 2 experiments. As determined using the above method, the compounds of Examples 1-116 exhibited a plC₅₀ greater than 4.0. For instance, the compounds of Example 2 and Example 39 each inhibited RIP2 kinase in the above method with a mean plC₅₀ of 5.

### FLAG His Tagged RIPK2 Preparation:

Full-length human RIPK2 (receptor-interacting serine-threonine kinase 2) cDNA was purchased from Invitrogen (Carlsbad, Calif., USA, Clone ID: IOH6368, RIPK2-pENTR 221). Gateway® LR cloning was used to site-specifically recombine RIPK2 downstream to an N-terminal FLAG-6His contained within the destination vector pDEST8-FLAG-His6 according to the protocol described by Invitrogen. Transfection into Spodoptera frugiperda(Sf9) insect cells was performed using Cellfectin® (Invitrogen), according to the manufacturer's protocol.

Sf9 cells were grown in Excell 420 (SAFC Biosciences, Lenexa, Kans., US; Andover, Hampshire UK) growth media at 27° C., 80 rpm in shake flask until of a sufficient volume to inoculate a bioreactor. The cells were grown in a 50 litre working volume bioreactor (Applikon, Foster City, Calif., US; Schiedam, Netherlands) at 27° C., 30% dissolved oxygen and an agitation rate of 60-140 rpm until the required volume was achieved with a cell concentration of approximately 3.7×e6 cells/ml. The insect cells were infected with Baculovirus at a multiplicity of infection (M01) of 12.7. The cultivation was continued for a 43 hour expression phase. The infected cells were removed from the growth media by centrifugation at 2500 g using a Viafuge (Carr) continuous centrifuge at a flow rate of 80 litres/hour. The cell pellet was immediately frozen and subsequently supplied for purification.

9.83×10¹⁰ Insect cells were re-suspended in 1.4 L lysis buffer (50 mM Tris (pH 8.0), 150 mM NaCl, 0.5 mM NaF, 0.1% Triton X-100, 1 mL/litre Protease Inhibitor Cocktail Set III (available from EMD Group; CalBiochem/Merck Biosciences, Gibbstown, N.J., US; Damstadt, Germany) and processed by dounce homogenization on ice. The suspension was then clarified by centrifugation at 47,900 g for 2 hours, at 4° C. The lysate was decanted from the insoluble pellet and loaded at a linear flow rate of 16 cm/h onto a 55 mL FLAG-M2 affinity column (2.6×10.4 cm) that had been pre-equilibrated with 10 column volumes buffer A (50 mM Tris (pH 8.0), 150 mM NaCl, 0.5 mM NaF, 1 mL/litre Protease Inhibitor Cocktail Set III). The column was then washed with 15 column volumes buffer A, and eluted with 6 column volumes buffer B (buffer A+150 μg/mL 3×FLAG peptide) at a linear flow rate of 57 cm/h. Fractions identified by SDS-PAGE as containing protein of interest were dialyzed to remove the 3×FLAG peptide from the preparation against 5 L of Buffer A (not containing the Protease Inhibitor Cocktail) overnight, using 10 kDa MWCO SnakeSkin Pleated Dialysis Tubing. The purification process yielded 11.3 mg of total protein, with the RIPK2 present at 40% purity by gel densitometry scanning, and identity confirmed by peptide mass fingerprinting. The main contaminating proteins in the preparation were identified as lower molecular weight degraded species of RIPK2.

### Fluorescent Ligand Preparation:

### 2-Methyl-5-(2-propen-1-yloxy)aniline

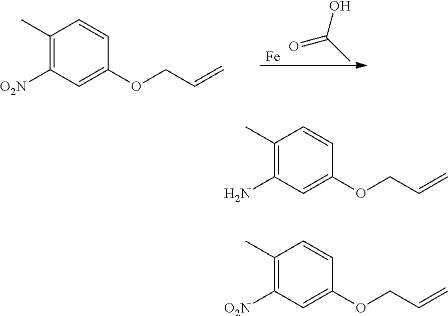

1-Methyl-2-nitro-4-(2-propen-1-yloxy)benzene (25.2 g, 130 mmol) was dissolved in ethanol (280 ml), water (28 ml), and acetic acid (5.6 ml, 98 mmol). Iron (29.1 g, 522 mmol) was added in six portions. The reaction was stirred for 72 hours, and then additional acetic acid (5.6 ml, 98 mmol) and 4 eq. of iron were added. The mixture was filtered through celite rinsing with EtOH and water and the filtrates were concentrated to remove EtOH. Diethylether (300 mL) was added along with 100 mL of 2 N HCl. The layers were separated and the ether layer was extracted with 2×100 mL of 2 N HCl. The acidic aqueous layer was slowly made pH 9 with NaOH pellets, and then dichloromethane (DCM, 300 mL) was added. The resulting emulsion was filtered using a Buchner funnel. The layers were separated and the aqueous layer extracted with DCM (2×100 mL). The combined extracts were dried over MgSO₄), filtered, and concentrated to a dark red oil (15.2 g). The crude material was purified via flash chromatography using a 120 g silica cartridge eluting with 5-15% EtOAc/hexanes for 30 min then 15-30% EtOAc/hexanes for 10 min. to give the titled compound as a red oil. MS (m/z) ¹H NMR (400 MHz, CHLOROFORM-d) δ ppm 2.23 (s, 3H) 4.51 (dt, J=5.29, 1.51 Hz, 2H) 5.29 (dd, J=10.45, 1.38 Hz, 1H) 5.38-5.46 (m, 1H) 5.99-6.12 (m, 1H) 6.01-6.10 (m, 1H) 6.46 (dd, J=8.31, 2.52 Hz, 1H) 6.56 (d, J=2.52 Hz, 1H) 7.01 (d, J=8.56 Hz, 1H); 164 (M+H⁺).

### 2-Chloro-N-[2-methyl-5-(2-propen-1-yloxy)phenyl]-4-pyrimidinamine

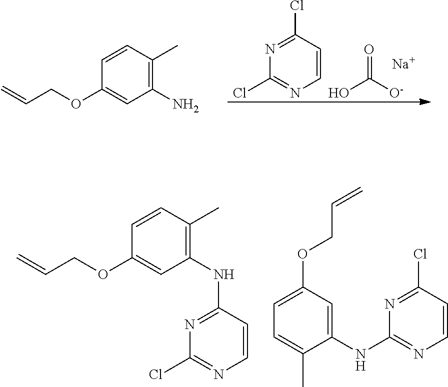

2-Methyl-5-(2-propen-1-yloxy)aniline (11.8 g, 72.3 mmol) was dissolved in tert-butanol (103 ml) and 2,4-dichloropyrimidine (10.77 g, 72.3 mmol) was added followed by sodium bicarbonate (18.22 g, 217 mmol). The reaction was heated at 80° C. for 17 hrs then additional 1,4-dichloropyrimidine (5.38 g, 36.6 mmol) was added and the reaction was stirred for 6 days. Additional 2,4-dichloropyrimidine (2.69 g, 17.8 mmol) was added and the reaction stirred for 2 days. The reaction was cooled to room temp diluting with EtOAc (200 mL) and water (200 mL). The layers were separated and the aqueous layer extracted with EtOAc (2×100 mL). The combined extracts were washed with brine (100 mL), dried over Na₂SO₄, filtered, and concentrated. The crude material was purified via flash chromatography using a 330 g silica cartridge eluting with 1-20% EtOAc/hexanes for 30 min then 20% EtOAc/hexanes for 50 min to give the titled compound (15.1 g). ¹H NMR (400 MHz, CHLOROFORM-d) δ ppm 2.20 (s, 3H) 4.54 (d, J=5.29 Hz, 2H) 5.32 (dd, J=10.45, 1.38 Hz, 1H) 5.42 (dd, J=17.37, 1.51 Hz, 1H) 5.99-6.12 (m, 1H) 6.35 (d, J=5.79 Hz, 1H) 6.83 (dd, J=8.44, 2.64 Hz, 1H) 6.89 (d, J=2.52 Hz, 6H) 7.14 (br. s., 6H) 7.21 (d, J=8.56 Hz, 7H) 8.10 (d, J=5.79 Hz, 6H); MS (m/z) 276 (M+H⁺).

### 3-[(4-{[2-Methyl-5-(2-propen-1-yloxy)phenyl]amino}-2-pyrimidinyl)amino]benzoic acid

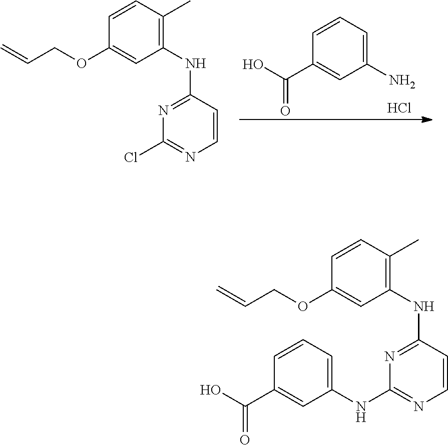 [SMILES: C=CCOc1ccc(C)c(Nc2ccnc(Cl)n2)c1 ; Nc1cccc(C(=O)O)c1 ; Cl ; C=CCOc1ccc(C)c(Nc2ccnc(Nc3cccc(C(=O)O)c3)n2)c1]

2-Chloro-N-[2-methyl-5-(2-propen-1-yloxy)phenyl]-4-pyrimidinamine (8 g, 29.0 mmol), 3-aminobenzoic acid (3.98 g, 29.0 mmol), and HCl (14.51 ml, 29.0 mmol) were dissolved in acetone (58.0 ml) and water (58.0 ml). The reaction was heated to 60° C. for 48 hrs. The reaction was cooled to room temperature passing air over it and a solid crashed out. Water (150 mL) was added and the solid was filtered washing with 3×50 mL water. The solid was dried in the vacuum funnel overnight affording the desired compound (10.9 g). ¹H NMR (400 MHz, METHANOL-d₄) δ ppm 2.21 (s, 3H) 4.47 (d, J=5.04 Hz, 2H) 5.24 (dd, J=10.58, 1.51 Hz, 1H) 5.37 (dd, J=17.25, 1.64 Hz, 1H) 5.97-6.09 (m, 4H) 6.29-6.39 (m, 1H) 6.89 (dd, J=8.44, 2.64 Hz, 4H) 6.96 (d, J=2.77 Hz, 1H) 7.04 (none, 0H) 7.23 (d, J=8.56 Hz, 1H) 7.34-7.41 (m, 1H) 7.75-7.79 (m, 1H) 7.81 (s, 1H) 7.85 (d, J=7.30 Hz, 3H) 7.98-8.09 (m, 3H); MS (m/z) 377 (M+H⁺).

### 1,1-Dimethylethyl {2-[({3-[(4-{[2-methyl-5-(2-propen-1-yloxy)phenyl]amino}-2-pyrimidinyl)amino]phenyl}carbonyl)amino]ethyl}carbamate

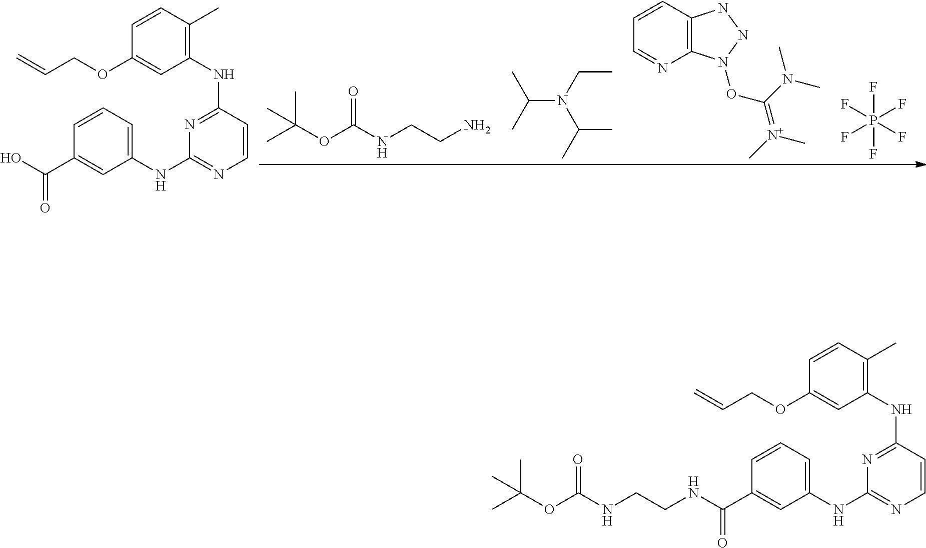

A solution of 3-[(4-{[2-methyl-5-(2-propen-1-yloxy)phenyl]amino}-2-pyrimidinyl)amino]benzoic acid (6.83 g, 18.15 mmol) and DIEA (9.51 ml, 54.4 mmol) in N,N-Dimethylformamide (DMF) (51.8 ml). was treated with N-(2-aminoethyl)carbamic acid tert-butyl ester (3.20 g, 19.96 mmol) and HATU (8.28 g, 21.77 mmol). EtOAc/Et₂O (400 mL, 1:1) was added and the layers separated. The organic layer was washed with water (3×300 mL) and brine (100 mL), dried over Na₂SO₄, filtered, and concentrated to give the titled compound (8.3 g). ¹H NMR (400 MHz, DMSO-d₆) δ ppm 1.38 (s, 9H) 2.15 (s, 3H) 3.09 (q, J=6.19 Hz, 2H) 3.27 (q, J=6.19 Hz, 2H) 4.51 (d, J=5.27 Hz, 2H) 5.24 (dd, J=10.54, 1.51 Hz, 1H) 5.37 (dd, J=17.32, 1.76 Hz, 1H) 6.02 (m, J=17.29, 10.51, 5.18, 5.18 Hz, 1H) 6.13 (d, J=5.77 Hz, 1H) 6.73 (dd, J=8.41, 2.63 Hz, 1H) 6.90 (t, J=5.65 Hz, 1H) 7.09 (d, J=2.51 Hz, 1H) 7.15 (d, J=8.28 Hz, 1H) 7.17-7.22 (m, 1H) 7.28 (d, J=7.78 Hz, 1H) 7.94-7.99 (m, 2H) 7.99-8.05 (m, 2H) 8.26 (t, J=5.65 Hz, 1H) 8.66 (s, 1H) 9.17 (s, 1H); MS (m/z) 519 (M+H⁺).

### 1,1-Dimethylethyl[2-({[3-({4-[(5-hydroxy-2-methylphenyl)amino]-2-pyrimidinyl}amino)phenyl]carbonyl}amino)ethyl]carbamate

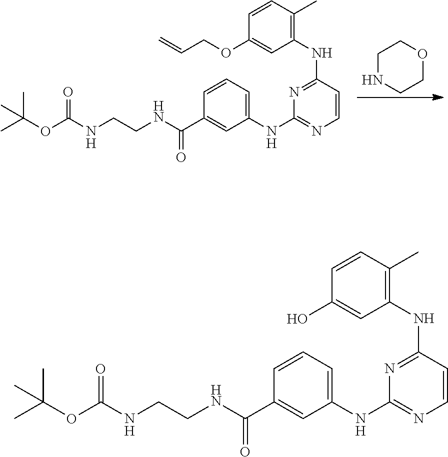 [SMILES: C=CCOc1ccc(C)c(Nc2ccnc(Nc3cccc(C(=O)NCCNC(=O)OC(C)(C)C)c3)n2)c1 ; C1COCCN1 ; Cc1ccc(O)cc1Nc1ccnc(Nc2cccc(C(=O)NCCNC(=O)OC(C)(C)C)c2)n1]

1,1-Dimethylethyl {2-[({3-[(4-{[2-methyl-5-(2-propen-1-yloxy)phenyl]amino}-2-pyrimidinyl)amino]phenyl}carbonyl)amino]ethyl}carbamate (5.5 g, 10.61 mmol) and morpholine (1.016 ml, 11.67 mmol) were dissolved in N,N-dimethylformamide (DMF) (42.4 ml) The atmosphere was exchanged for nitrogen and then it was treated with Tetrakis (1.226 g, 1.061 mmol). The reaction was heated to 80° C. for 3 hrs. The reaction was diluted with EtOAc (250 mL) and washed with water (3×200 mL) then brine (100 mL). The organic layer was dried over Na2SO4, filtered, and concentrated to about 50 mL and let stand overnight. A solid formed and to the suspension was added 50 mL ether. The solid was filtered washing with ether to give the desired product as an orange solid (4.75 g). ¹H NMR (400 MHz, METHANOL-d₄) δ ppm 1.42 (s, 9H) 2.17 (s, 3H) 3.29 (t, J=6.04 Hz, 2H) 3.46 (t, J=6.17 Hz, 2H) 6.04 (d, J=6.04 Hz, 1H) 6.65 (dd, J=8.31, 2.52 Hz, 1H) 6.87 (d, J=2.52 Hz, 1H) 7.09 (d, J=8.31 Hz, 1H) 7.27-7.33 (m, 1H) 7.35-7.41 (m, 1H) 7.53-7.61 (m, 1H) 7.62-7.70 (m, 2H) 7.75 (d, J=8.06 Hz, 1H) 7.91 (d, J=6.04 Hz, 1H) 8.11 (s, 1H); MS (m/z) 479 (M+H⁺).

### N-(2-Aminoethyl)-3-({4-[(5-hydroxy-2-methylphenyl)amino]-2-pyrimidinyl}amino)benzamide

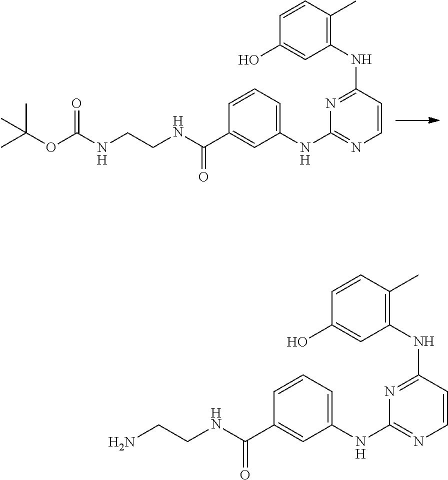 [SMILES: Cc1ccc(O)cc1Nc1ccnc(Nc2cccc(C(=O)NCCNC(=O)OC(C)(C)C)c2)n1 ; Cc1ccc(O)cc1Nc1ccnc(Nc2cccc(C(=O)NCCN)c2)n1]

1,1-Dimethylethyl[2-({[3-({4-[(5-hydroxy-2-methylphenyl)amino]-2-pyrimidinyl}amino)phenyl]carbonyl}amino)ethyl]carbamate (4.75 g, 8.93 mmol) (contaminated with tetrakis or related entities) was dissolved in dichloromethane (DCM) (28.6 ml) and trifluoroacetic acid (TFA) (7.15 ml). The reaction concentrated to give the desired product as the TFA salt containing the same impurities going into the reaction (6.5 g) MS (m/z) 379 (M+H⁺).

### 5-({[2-({[3-({4-[(5-Hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid

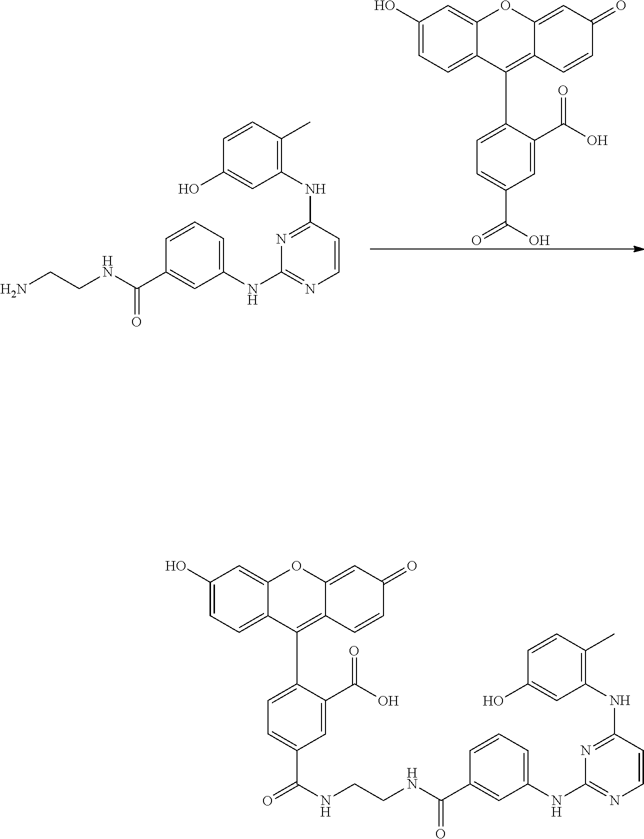 [SMILES: Cc1ccc(O)cc1Nc1ccnc(Nc2cccc(C(=O)NCCN)c2)n1 ; O=C(O)c1ccc(-c2c3ccc(=O)cc-3oc3cc(O)ccc23)c(C(=O)O)c1 ; Cc1ccc(O)cc1Nc1ccnc(Nc2cccc(C(=O)NCCNC(=O)c3ccc(-c4c5ccc(=O)cc-5oc5cc(O)ccc45)c(C(=O)O)c3)c2)n1]

To a suspension of N-(2-aminoethyl)-3-({4-[(5-hydroxy-2-methylphenyl)amino]-2-pyrimidinyl}amino)benzamide (1 g, 1.319 mmol) in N,N-dimethylformamide (DMF) (13.19 ml) was added 5-FAM (5-carboxyfluorescein single isomer) (0.397 g, 1.055 mmol), triethylamine (0.919 ml, 6.60 mmol), EDC (0.506 g, 2.64 mmol), and HOBT (0.202 g, 1.319 mmol). The reaction was stirred overnight then the pH was adjusted to 3 with 2 N HCl. The solution was extracted with EtOAc (100 mL) and the organic layer washed with water (1×50 mL), dried over Na₂SO₄, filtered, and concentrated to give the titled compound. MS (m/z) 737 (M+H⁺).

### Biological In Vivo Assay

The efficacy of the RIP2 inhibitors of this invention may also be evaluated in vivo in rodents. Intraperitoneal (i.p.) or intravenous (i.v.) administration of L18-MDP in mice has been shown to induce an inflammatory response through activation of the NOD2 signaling pathway (Rosenweig, H. L., et al. 2008. Journal of Leukocyte Biology 84:529-536). The level of the inflammatory response in the L18-MDP treated mice/rats is monitored using conventional techniques by measuring increases in cytokine levels (IL8, TNFα, IL6 and IL-1β) in serum and/or peritoneal lavage fluid and by measuring neutrophil influx into the peritoneal space (when L18-MDP is dosed i.p.). Inhibition of the L18-MDP induced inflammatory response in treated rodents may be shown by orally pre-dosing with selected compounds of this invention, then measuring and comparing cytokine levels (IL8, TNFα, IL6 and IL-1β) in serum and/or peritoneal lavage fluid and neutrophil influx into the peritoneal space (when L18-MDP is dosed i.p.) using conventional techniques.

1-31. (canceled)

32. A compound according to Formula (I): 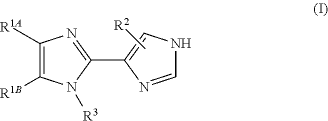
wherein:
R^{1A} and R^{1B} are each independently selected from H and a (C₁-C₆)alkyl group,
or R^{1A} and R^{1B}, taken together with the atoms through which they are attached, form a 6-membered non-aromatic carbocyclic ring or an optionally substituted 6-membered aromatic carbocyclic or heterocyclic ring,
wherein the 6-membered aromatic heterocyclic ring contains one or two nitrogen heteroatoms, and the 6-membered aromatic carbocyclic or heterocyclic ring is optionally substituted by 1-3 substituents each independently selected from halogen, cyano, (C₁-C₄)alkyl, halo(C₁-C₆)alkyl, hydroxy(C₁-C₆)alkyl, (C₁-C₄)alkoxy, halo(C₁-C₆)alkoxy, (C₁-C₄ alkyl)amino-, hydroxy(C₂-C₄ alkyl)amino-, (C₁-C₆ alkyl)(C₁-C₄ alkyl)amino-, (hydroxy(C₂-C₄ alkyl))(C₁-C₄ alkyl)amino-, —CO₂H, —CO₂(C₁-C₄alkyl), —CONH₂, —C ONH(C₁-C₄ alkyl), —CON(C₁-C₄ alkyl)(C₁-C₆ alkyl), —CONH(aryl), —CONH(heteroaryl), —SO₂NH₂, —SO₂NH(C₁-C₄ alkyl), —SO₂NH(—C₁-C₄ alkyl-phenyl), —SO₂N(C₁-C₄alkyl)(C₁-C₆alkyl), heterocycloalkyl, —CO(heterocycloalkyl), and —SO₂(heterocycloalkyl),
wherein any of said heterocycloalkyl is optionally substituted by 1-3 substituents each independently selected from hydroxy, (C₁-C₆)alkyl and hydroxy(C₁-C₄)alkyl,
any of said (C₁-C₄alkyl) is optionally substituted by 1-3 substituents each independently selected from (C₁-C₆)alkoxy, heterocycloalkyl, amino, (C₁-C₄ alkyl)amino-, and (C₁-C₄ alkyl)(C₁-C₄ alkyl)amino-, and
said aryl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy;
R² is monocyclic or bicyclic aryl or monocyclic or bicyclic heteroaryl, optionally substituted by one, two or three R^{2A} substituents,
wherein each R^{2A} is independently selected from halogen, cyano, (C₁-C₄)alkyl, halo(C₁-C₄)alkyl, C₁-C₄ alkoxy, hydroxyl, —CO₂H, —CO₂(C₁-C₄)alkyl, —CONH₂, —CONH(C₁-C₄alkyl), —CON(C₁-C₄alkyl)(C₁-C₄alkyl), phenylC₁-C₄ alkoxy, C₁-C₄ alkylthio-, —SO₂(C₁-C₄)alkyl, —SO₂NH₂, —SO₂NH(C₁-C₄alkyl), —SO₂N(C₁-C₄alkyl)(C₁-C₄alkyl), and monocyclic or bicyclic heteroaryl optionally substituted by (C₁-C₄)alkyl;
R³ is (C₁-C₄)alkyl, (C₁-C₂)alkoxy(C₁-C₄)alkyl-, hydroxy(C₂-C₄)alkyl-, 5-6 membered heterocycloalkyl, 5-6 membered heterocycloalkyl(C₁-C₄)alkyl-, or 5-6 membered heteroaryl(C₁-C₄)alkyl-;
provided the compound is not 1-[2-(methyloxy)ethyl]-5′-phenyl-1H,1′H-2,4′-biimidazole;
or a salt thereof.

33. The compound or salt according to claim 32, wherein R^{1A} and R^{1B}, taken together with the atoms through which they are attached form a 6-membered aromatic carbocyclic or heterocyclic ring, to provide Formula (I-B): 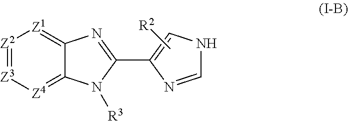
wherein:
each Z¹, Z², Z³, and Z⁴ is independently selected from CH and CR¹; or any one or two of Z¹, Z², Z³, and Z⁴ is N, and each of the remaining two or three of Z¹, Z², Z³, and Z⁴ are independently selected from CH and CR¹;
each R¹ is independently selected from halogen, cyano, (C₁-C₄)alkyl, halo(C₁-C₆)alkyl, hydroxy(C₁-C₄)alkyl, (C₁-C₄)alkoxy, halo(C₁-C₄)alkoxy, (C₁-C₄ alkyl)amino-, hydroxy(C₂-C₄ alkyl)amino-, (C₁-C₄)alkoxy-(C₂-C₄ alkyl)amino-, (C₁-C₄ alkyl)(C₁-C₄ alkyl)amino-, (hydroxy(C₂-C₄ alkyl))(C₁-C₄ alkyl)amino-, ((C₁-C₄)alkoxy-(C₂-C₄ alkyl))(C₁-C₄ alkyl)amino-, —CO₂H, —CO₂ (C₁-C₄alkyl), heterocycloalkyl, —CO(heterocycloalkyl), —SO₂(heterocycloalkyl), —CONH₂, alkyl)HNCO—, ((C₁-C₄)alkoxy-(C₂-C₄)alkyl)HNCO—, (heterocycloalkyl-(C₁-C₄)alkyl)HNCO—, (C₁-C₄ alkyl)(C₁-C₄ alkyl)NCO—, ((C₁-C₄)alkoxy-(C₂-C₄)alkyl)(C₁-C₄ alkyl)NCO—, —CONH(phenyl), —CONH(heteroaryl), —SO₂NH₂, (C₁-C₄ alkyl)HNSO₂—, ((C₁-C₄)alkoxy-(C₂-C₄)alkyl)HNSO₂—, (phenyl(C₁-C₄)alkyl)HNSO₂—, (C₁-C₄alkyl)(C₁-C₄ alkyl)NSO₂—, and ((C₁-C₄)alkoxy-(C₂-C₄)alkyl)(C₁-C₄alkyl)NSO₂—,
said heteroaryl is a 5-6 membered aromatic ring containing one heteroatom selected from N, O and S, or containing one nitrogen atom and one additional heteroatom selected from N, O and S, said phenyl or heteroaryl is optionally substituted by 1-3 substituents each independently selected from halogen, (C₁-C₄)alkyl, halo(C₁-C₄)alkyl, (C₁-C₄)alkoxy, and halo(C₁-C₄)alkoxy, and
any of said heterocycloalkyl is a 4-7 membered non-aromatic ring containing one heteroatom selected from N, O and S, or containing one nitrogen atom and one additional heteroatom selected from N, O and S; which heterocycloalkyl is optionally substituted by 1-2 substituents each independently selected from hydroxy, (C₁-C₆)alkyl and hydroxy(C₁-C₄)alkyl;

34. The compound or salt according to claim 33, wherein each R¹ is independently selected from halogen, (C₁-C₄)alkyl, halo(C₁-C₆)alkyl, (C₁-C₄)alkoxy, halo(C₁-C₆)alkoxy, —CO₂H, —CO₂ (C₁-C₄alkyl), —CONH₂, —CONH(C₁-C₄alkyl), —CON(C₁-C₄alkyl)(C₁-C₆alkyl), —CONH(aryl), —CONH(heteroaryl), —SO₂NH₂, —SO₂NH(C₁-C₄alkyl), and —SO₂N(C₁-C₄alkyl)(C₁-C₆alkyl),
wherein any of said (C₁-C₄alkyl) is optionally substituted by one to three groups each independently selected from (C₁-C₆)alkoxy, heterocycloalkyl, amino, (C₁-C₄ alkyl)amino-, and (C₁-C₄ alkyl)(C₁-C₄ alkyl)amino-,
and said aryl or heteroaryl is optionally substituted by one to three groups each independently selected from halogen, (C₁-C₄)alkyl, halo(C₁-C₄)alkyl, (C₁-C₄)alkoxy, and halo(C₁-C₄)alkoxy.

35. The compound or salt according to claim 33, wherein:
each of Z¹, Z², Z³, and Z⁴ is CH; or
one of Z² or Z³ is CR¹ and Z¹, Z⁴ and the other of Z² or Z³ is CH; or
Z² and Z³ are CR¹ and Z¹ and Z⁴ are CH.

36. The compound or salt according to claim 33, wherein:
any one of Z¹, Z², Z³, and Z⁴ is N, and each of the remaining three of Z¹, Z², Z³, and Z⁴ is CH; or
any one of Z¹, Z², Z³, and Z⁴ is N, one of the remaining Z¹, Z², Z³, and Z⁴ is CR¹; and the remaining two of Z¹, Z², Z³, and Z⁴ is CH; or
Z² is N, Z¹ is CR¹ and Z³, and Z⁴ are CH; or Z² is N, Z¹ and Z³ are CR¹ and Z⁴ is CH; or Z⁴ is N, Z³ is CR¹ and Z¹ and Z² are CH.

37. The compound or salt according to claim 33, wherein each R¹ is independently selected from halogen, cyano, (C₁-C₄)alkyl, halo(C₁-C₆)alkyl, hydroxy(C₁-C₄)alkyl, (C₁-C₄)alkoxy, (hydroxy(C₂-C₄ alkyl))(C₁-C₄ alkyl)amino-, ((C₁-C₄)alkoxy-(C₂-C₄ alkyl))(C₁-C₄ alkyl)amino-, —CO₂H, —CO₂ (C₁-C₄ alkyl), heterocycloalkyl, —CO(heterocycloalkyl), —SO₂(heterocycloalkyl), (C₁-C₄ alkyl)HNCO—, ((C₁-C₄)alkoxy-(C₂-C₄)alkyl)HNCO—, (hetero cyclo alkyl-(C₁-C₄)alkyl)HNCO—, (C₁-C₄ alkyl)(C₁-C₄ alkyl)NCO—, —C ONH(hetero aryl), —SO₂NH₂, (C₁-C₄ alkyl)HNS₂—, ((C₁-C₄)alkoxy-(C₂-C₄)alkyl)HNSO₂—, and (phenyl(C₁-C₄)alkyl)HNSO₂₅
wherein said heteroaryl is a 6-membered aromatic heterocyclic ring containing one or two nitrogen heteroatoms which ring is optionally substituted by 1-2 substituents each independently selected from halogen, (C₁-C₄)alkyl and (C₁-C₄)alkoxy, and
said heterocycloalkyl is a 4-6 membered non-aromatic heterocyclic ring containing one heteroatom selected from N, O and S, or containing one nitrogen atoms and one additional heteroatom selected from N, O and S, which ring is optionally substituted by 1-2 substituents each independently selected from hydroxy, (C₁-C₆)alkyl and hydroxy(C₁-C₄)alkyl.

38. The compound or salt according to claim 33, wherein each R¹ is methyl, chloro, fluoro, bromo, cyano, trifluoromethyl, methoxy, ethoxy, —CH₂OH, —C(CH₃)₂OH, —CO₂H, —CO₂CH₃, —SO₂CH₃, —SO₂-benzyl, —SO₂NH₂, —CONHCH₃, —CON(CH₃)₂, —CONHCH₂CH₂OCH₃, —CONHCH₂CH₂(morpholin-4-yl), —CONH-pyrid-2-yl, —CONH-pyrid-3-yl, —CONH-pyrid-4-yl, morpholin-4-yl-CO—, [(3R)-3-methyl-morpholin-4-yl]-CO—, [(3S)-3-methyl-morpholin-4-yl]-CO—, (4-methyl-piperazin-1-yl)-CO—, morpholin-4-yl, (3S)-3-methyl-morpholin-4-yl, (3R)-3-methyl-morpholin-4-yl, (3R)-3-ethyl-morpholin-4-yl, 2-methyl-morpholin-4-yl, ((2S,5R)-5-ethyl-2-hydroxymethyl-morpholin-4-yl, ((2S,5S)-5-methyl-2-hydroxymethyl-morpholin-4-yl, piperidin-1-yl, pyrrolidin-1-yl, azetidin-1-yl, 4-methyl-piperazin-1-yl, (3S)-3,4-dimethyl-piperazin-1-yl, (2-(methoxy)ethyl)(methyl)amino-, (2-(methoxy)ethyl)(ethyl)amino-, (2-(hydroxyl)ethyl)(methyl)amino-, morpholin-4-yl-SO₂—, pyrrolidin-1-yl-SO₂—, piperazin-1-yl-SO₂—, 4-methyl-piperazin-1-yl-SO₂—, and (2-(methoxy)ethyl)HNSO₂—.

39. The compound or salt according to claim 33, wherein R² is an optionally substituted phenyl, an optionally substituted 5-6 membered, monocyclic or 9-10 membered, bicyclic heteroaryl, wherein said heteroaryl contains one heteroatom selected from N, O and S, or contains one or two nitrogen atoms and one additional heteroatom selected from N, O and S.

40. The compound or salt according to claim 32, wherein R² is phenyl, pyridyl, benzothiazolyl, indolyl, indazolyl, or benzoxadiazolyl, each optionally substituted by one R^{2A} and further optionally substituted by a second R^{2A}.

41. The compound or salt according to claim 33, wherein R² is phenyl, pyridyl, benzothiazolyl, indolyl, indazolyl, or benzoxadiazolyl, each optionally substituted by one R^{2A} and further optionally substituted by a second R^{2A}.

42. The compound or salt according claim 33, wherein each R^{2A} is independently selected from halogen, (C₁-C₄)alkyl, halo(C₁-C₄)alkyl, hydroxyl, C₁-C₄ alkoxy, —CONH₂, —CONH(C₁-C₄alkyl), —CON(C₁-C₄alkyl)(C₁-C₄alkyl), —SO₂(C₁-C₄)alkyl, —SO₂NH₂, —SO₂NH(C₁-C₄alkyl), and a 5-6 membered heteroaryl optionally substituted by (C₁-C₄)alkyl.

43. The compound or salt according to claim 32, wherein each R^{2A} is independently selected from chloro, fluoro, methyl, trifluoromethyl, hydroxyl, methoxy, —CON(CH₃)₂, —CONH₂, —SO₂NH₂ and —SO₂CH₃.

44. The compound or salt according to claim 33, wherein each R^{2A} is independently selected from chloro, fluoro, methyl, trifluoromethyl, hydroxyl, methoxy, —CON(CH₃)₂, —CONH₂, —SO₂NH₂ and —SO₂CH₃.

45. The compound or salt according to claim 33, wherein R² is an unsubstituted benzothiazolyl, indolyl, indazolyl or benzoxadiazolyl, or R² is an optionally substituted phenyl group, optionally substituted by one R^{2A} selected from chloro, fluoro, methoxy, —CON(CH₃)₂, —CONH₂, —SO₂NH₂, and —SO₂CH₃, and further optionally substituted by a second R^{2A} selected from chloro, fluoro, and methoxy.

46. The compound or salt according to claim 33, wherein R³ is —CH₂CH₂CH₃, —CH₂CH₂OCH₃, —CH₂CH₂OCH₂CH₃, —CH₂CH₂OH, tetrahydrofuran-3-yl, —CH₂-tetrahydrofuran-2-yl, —CH₂-pyrid-2-yl, or —CH₂CH₂-pyrid-2-yl.

47. The compound or salt according to claim 32, wherein R³ is —CH₂CH₂OCH₃.

48. The compound or salt according to claim 33, wherein R³ is —CH₂CH₂OCH₃.

49. A compound which is:
2-[2-(4-chlorophenyl)-1H-imidazol-4-yl]-1-[2-(methyloxy)ethyl]-N-[2-(4-morpholinyl)ethyl]-1H-benzimidazole-5-carboxamide,
2-[2-(4-chlorophenyl)-1H-imidazol-4-yl]-N-methyl-1-(2-pyridinylmethyl)-1H-benzimidazole-5-carboxamide,
2-[2-(4-chlorophenyl)-1H-imidazol-4-yl]-N-methyl-1-(tetrahydro-2-furanylmethyl)-1H-benzimidazole-5-carboxamide,
2-[2-(4-chlorophenyl)-1H-imidazol-4-yl]-N-methyl-1-(tetrahydro-3-furanyl)-1H-benzimidazole-5-carboxamide,
2-[2-(4-chlorophenyl)-1H-imidazol-4-yl]-N-methyl-1-[2-(2-pyridinyl)ethyl]-1H-benzimidazole-5-carboxamide,
2-{2-[2-fluoro-5-(methyloxy)phenyl]-1H-imidazol-4-yl}-N-methyl-1-[2-(methyloxy)ethyl]-1H-benzimidazole-5-carboxamide,
N-methyl-1-[2-(methyloxy)ethyl]-2-{2-[3-(methyloxy)phenyl]-1H-imidazol-4-yl}-1H-benzimidazole-5-carboxamide,
2-{2-[2,5-bis(methyloxy)phenyl]-1H-imidazol-4-yl}-N-methyl-1-[2-(methyloxy)ethyl]-1H-benzimidazole-5-carboxamide,
N-methyl-1-[2-(methyloxy)ethyl]-2-{2-[2-(methyloxy)phenyl]-1H-imidazol-4-yl}-1H-benzimidazole-5-carboxamide,
N-methyl-1-[2-(methyloxy)ethyl]-2-{2-[4-(methyloxy)phenyl]-1H-imidazol-4-yl}-1H-benzimidazole-5-carboxamide,
2-{2-[4-chloro-3-(methyloxy)phenyl]-1H-imidazol-4-yl}-N,N-dimethyl-1-[2-(methyloxy)ethyl]-1H-benzimidazole-5-carboxamide,
2-[2-(4-chloro-3-hydroxyphenyl)-1H-imidazol-4-yl]-N,N-dimethyl-1-[2-(methyloxy)ethyl]-1H-benzimidazole-5-carboxamide,
2-[2-(1,3-benzothiazol-5-yl)-1H-imidazol-4-yl]-N-methyl-1-[2-(methyloxy)ethyl]-1H-benzimidazole-5-carboxamide,
2-{2-[4-chloro-3-(methyloxy)phenyl]-1H-imidazol-4-yl}-N-methyl-1-[2-(methyloxy)ethyl]-1H-benzimidazole-5-carboxamide,
N-methyl-1-[2-(methyloxy)ethyl]-2-{2-[4-(methylsulfonyl)phenyl]-1H-imidazol-4-yl}-1H-benzimidazole-5-carboxamide,
2-{2-[4-(aminosulfonyl)phenyl]-1H-imidazol-4-yl}-N-methyl-1-[2-(methyloxy)ethyl]-1H-benzimidazole-5-carboxamide,
2-[2-(1H-indol-5-yl)-1H-imidazol-4-yl]-N-methyl-1-[2-(methyloxy)ethyl]-1H-benzimidazole-5-carboxamide,
2-[2-(1H-indol-6-yl)-1H-imidazol-4-yl]-N-methyl-1-[2-(methyloxy)ethyl]-1H-benzimidazole-5-carboxamide,
2-{2-[3-(amino sulfonyl)phenyl]-1H-imidazol-4-yl}-N-methyl-1-[2-(methyloxy)ethyl]-1H-benzimidazole-5-carboxamide,
2-{2-[3-(amino carbonyl)phenyl]-1H-imidazol-4-yl}-N-methyl-1-[2-(methyloxy)ethyl]-1H-benzimidazole-5-carboxamide,
2-[2-(1H-indazol-5-yl)-1H-imidazol-4-yl]-N-methyl-1-[2-(methyloxy)ethyl]-1H-benzimidazole-5-carboxamide,
2-[2-(1H-indazol-6-yl)-1H-imidazol-4-yl]-N-methyl-1-[2-(methyloxy)ethyl]-1H-benzimidazole-5-carboxamide,
2-[2-(2,1,3-benzoxadiazol-5-yl)-1H-imidazol-4-yl]-N-methyl-1-[2-(methyloxy)ethyl]-1H-benzimidazole-5-carboxamide,
2-[2-(2,1,3-benzoxadiazol-5-yl)-1H-imidazol-4-yl]-N,N-dimethyl-1-[2-(methyloxy)ethyl]-1H-benzimidazole-5-carboxamide,
2-[2-(1,3-benzothiazol-5-yl)-1H-imidazol-4-yl]-N,N-dimethyl-1-[2-(methyloxy)ethyl]-1H-benzimidazole-5-carboxamide,
5-(4-{6-methyl-1-[2-(methyloxy)ethyl]-1H-benzimidazol-2-yl}-1H-imidazol-2-yl)-2,1,3-benzoxadiazole,
5-(4-{6-methyl-1-[2-(methyloxy)ethyl]-1H-benzimidazol-2-yl}-1H-imidazol-2-yl)-1,3-benzothiazole,
2-{2-[4-chloro-3-(methyloxy)phenyl]-1H-imidazol-4-yl}-6-methyl-1-[2-(methyloxy)ethyl]-1H-benzimidazole,
2-chloro-5-(4-{6-methyl-1-[2-(methyloxy)ethyl]-1H-benzimidazol-2-yl}-1H-imidazol-2-yl)phenol,
2-[2-(4-chlorophenyl)-1H-imidazol-4-yl]-1-[2-(methyloxy)ethyl]-1H-benzimidazole,
methyl 2-[2-(4-chlorophenyl)-1H-imidazol-4-yl]-1-[2-(methyloxy)ethyl]-1H-benzimidazole-5-carboxylate,
2-[2-(4-chlorophenyl)-1H-imidazol-4-yl]-N,1-bis[2-(methyloxy)ethyl]-1H-benzimidazole-5-carboxamide,
2-[2-(4-chlorophenyl)-1H-imidazol-4-yl]-N-methyl-1-[2-(methyloxy)ethyl]-1H-benzimidazole-5-carboxamide,
2-[2-(4-chlorophenyl)-1H-imidazol-4-yl]-N,N-dimethyl-1-[2-(methyloxy)ethyl]-1H-benzimidazole-5-carboxamide,
2-[2-(4-chlorophenyl)-1H-imidazol-4-yl]-1-[2-(methyloxy)ethyl]-5-(trifluoromethyl)-1H-benzimidazole,
2-[2-(4-chlorophenyl)-1H-imidazol-4-yl]-6-(methyloxy)-1-[2-(methyloxy)ethyl]-1H-benzimidazole,
2-[2-(4-chlorophenyl)-1H-imidazol-4-yl]-5-methyl-1-[2-(methyloxy)ethyl]-1H-benzimidazole,
2-[2-(4-chlorophenyl)-1H-imidazol-4-yl]-6-methyl-1-[2-(methyloxy)ethyl]-1H-benzimidazole,
N,1-bis[2-(methyloxy)ethyl]-2-(2-phenyl-1H-imidazol-4-yl)-1H-benzimidazole-5-carboxamide,
2-[2-(4-chlorophenyl)-1H-imidazol-4-yl]-1-(2-hydroxyethyl)-N-[2-(methyloxy)ethyl]-1H-benzimidazole-5-carboxamide,
2-[2-(4-chlorophenyl)-1H-imidazol-4-yl]-N-methyl-1-[2-(methyloxy)ethyl]-1H-benzimidazole-6-carboxamide,
2-[2-(4-chlorophenyl)-1H-imidazol-4-yl]-1-[2-(methyloxy)ethyl]-1H-benzimidazole-6-carboxylic acid,
2-[2-(3-chlorophenyl)-1H-imidazol-4-yl]-N-methyl-1-[2-(methyloxy)ethyl]-1H-benzimidazole-5-carboxamide,
2-[2-(4-chlorophenyl)-1H-imidazol-4-yl]-N-methyl-1-propyl-1H-benzimidazole-5-carboxamide,
2-[2-(4-chlorophenyl)-1H-imidazol-4-yl]-1-[2-(ethyloxy)ethyl]-N-methyl-1H-benzimidazole-5-carboxamide,
2-[2-(4-chlorophenyl)-1H-imidazol-4-yl]-7-methyl-1-[2-(methyloxy)ethyl]-1H-benzimidazole,
2-[2-(4-chlorophenyl)-1H-imidazol-4-yl]-1-[2-(methyloxy)ethyl]-N-4-pyridinyl-1H-benzimidazole-5-carboxamide,
2-[2-(4-chlorophenyl)-1H-imidazol-4-yl]-1-[2-(methyloxy)ethyl]-N-2-pyridinyl-1H-benzimidazole-6-carboxamide,
2-[2-(4-chlorophenyl)-1H-imidazol-4-yl]-1-[2-(methyloxy)ethyl]-N-3-pyridinyl-1H-benzimidazole-6-carboxamide,
2-[2-(4-chlorophenyl)-1H-imidazol-4-yl]-1-[2-(methyloxy)ethyl]-N-4-pyridinyl-1H-benzimidazole-6-carboxamide,
2-[2-(4-chlorophenyl)-1H-imidazol-4-yl]-1-[2-(methyloxy)ethyl]-N-2-pyridinyl-1H-benzimidazole-5-carboxamide,
2-[2-(4-chlorophenyl)-1H-imidazol-4-yl]-1-[2-(methyloxy)ethyl]-1H-imidazo[4,5-c]pyridine,
2-[2-(1,3-benzothiazol-5-yl)-1H-imidazol-4-yl]-1-[2-(methyloxy)ethyl]-1H-benzimidazole-6-carbonitrile,
5-{4-[6-methyl-1-[2-(methyloxy)ethyl]-5-(methylsulfonyl)-1H-benzimidazol-2-yl]-1H-imidazol-2-yl}-1,3-benzothiazole,
5-(4-{6-fluoro-1-[2-(methyloxy)ethyl]-1H-benzimidazol-2-yl}-1H-imidazol-2-yl)-1,3-benzothiazole,
5-(4-{7-chloro-1-[2-(methyloxy)ethyl]-1H-benzimidazol-2-yl}-1H-imidazol-2-yl)-1,3-benzothiazole,
5-(4-{4-methyl-1-[2-(methyloxy)ethyl]-1H-benzimidazol-2-yl}-1H-imidazol-2-yl)-1,3-benzothiazole,
5-(4-{6-chloro-1-[2-(methyloxy)ethyl]-1H-benzimidazol-2-yl}-1H-imidazol-2-yl)-1,3-benzothiazole,
5-(4-{6-bromo-1-[2-(methyloxy)ethyl]-1H-benzimidazol-2-yl}-1H-imidazol-2-yl)-1,3-benzothiazole,
5-(4-{6-(ethyloxy)-1-[2-(methyloxy)ethyl]-1H-benzimidazol-2-yl}-1H-imidazol-2-yl)-1,3-benzothiazole,
5-{4-[1-[2-(methyloxy)ethyl]-6-(trifluoromethyl)-1H-benzimidazol-2-yl]-1H-imidazol-2-yl}-1,3-benzothiazole,
2-[2-(1,3-benzothiazol-5-yl)-1H-imidazol-4-yl]-N,N,6-trimethyl-1-[2-(methyloxy)ethyl]-1H-benzimidazole-5-carboxamide,
5-{4-[6-methyl-1-[2-(methyloxy)ethyl]-5-(4-morpholinylcarbonyl)-1H-benzimidazol-2-yl]-1H-imidazol-2-yl}-1,3-benzothiazole,
5-(4-{6-methyl-5-{[(3R)-3-methyl-4-morpholinyl]carbonyl}-1-[2-(methyloxy)ethyl]-1H-benzimidazol-2-yl}-1H-imidazol-2-yl)-1,3-benzothiazole,
5-(4-{6-methyl-5-{[(3S)-3-methyl-4-morpholinyl]carbonyl}-1-[2-(methyloxy)ethyl]-1H-benzimidazol-2-yl}-1H-imidazol-2-yl)-1,3-benzothiazole,
5-(4-{6-methyl-1-[2-(methyloxy)ethyl]-5-[(4-methyl-1-piperazinyl)carbonyl]-1H-benzimidazol-2-yl}-1H-imidazol-2-yl)-1,3-benzothiazole,
methyl 2-[2-(1,3-benzothiazol-5-yl)-1H-imidazol-4-yl]-1-[2-(methyloxy)ethyl]-1H-benzimidazole-6-carboxylate,
2-[2-(1,3-benzothiazol-5-yl)-1H-imidazol-4-yl]-1-[2-(methyloxy)ethyl]-1H-benzimidazole-6-carboxylic acid,
methyl 2-[2-(1,3-benzothiazol-5-yl)-1H-imidazol-4-yl]-1-[2-(methyloxy)ethyl]-1H-benzimidazole-5-carboxylate,
2-{2-[2-(1,3-benzothiazol-5-yl)-1H-imidazol-4-yl]-1-[2-(methyloxy)ethyl]-1H-benzimidazol-5-yl}-2-propanol,
2-{2-[2-(1,3-benzothiazol-5-yl)-1H-imidazol-4-yl]-1-[2-(methyloxy)ethyl]-1H-benzimidazol-5-yl}-2-propanol,
2-[2-(1,3-benzothiazol-5-yl)-1H-imidazol-4-yl]-3-[2-(methyloxy)ethyl]-3H-imidazo[4,5-b]pyridine,
2-[2-(1,3-benzothiazol-5-yl)-1H-imidazol-4-yl]-5-methyl-3-[2-(methyloxy)ethyl]-3H-imidazo[4,5-b]pyridine,
2-[2-(1,3-benzothiazol-5-yl)-1H-imidazol-4-yl]-1-[2-(methyloxy)ethyl]-1H-imidazo[4,5-b]pyridine,
2-[2-(1,3-benzothiazol-5-yl)-1H-imidazol-4-yl]-6-methyl-3-[2-(methyloxy)ethyl]-3H-imidazo[4,5-b]pyridine,
{2-[2-(1,3-benzothiazol-5-yl)-1H-imidazol-4-yl]-1-[2-(methyloxy)ethyl]-1H-benzimidazol-5-yl}methanol,
5-{4-[1-[2-(methyloxy)ethyl]-5-(trifluoromethyl)-1H-benzimidazol-2-yl]-1H-imidazol-2-yl}-1,3-benzothiazole,
5-{4-[1-[2-(methyloxy)ethyl]-5-(methylsulfonyl)-1H-benzimidazol-2-yl]-1H-imidazol-2-yl}-1,3-benzothiazole,
2-[2-(1,3-benzothiazol-5-yl)-1H-imidazol-4-yl]-1-[2-(methyloxy)ethyl]-1H-benzimidazole-5-sulfonamide,
5-(4-{5-bromo-1-[2-(methyloxy)ethyl]-1H-benzimidazol-2-yl}-1H-imidazol-2-yl)-1,3-benzothiazole,
2-[2-(1,3-benzothiazol-5-yl)-1H-imidazol-4-yl]-1-[2-(methyloxy)ethyl]-1H-benzimidazole-5-carbonitrile,
2-[2-(1,3-benzothiazol-5-yl)-1H-imidazol-4-yl]-6-chloro-1-[2-(methyloxy)ethyl]-1H-benzimidazole-5-carboxylic acid,
5-(4-{1-[2-(methyloxy)ethyl]-1H-benzimidazol-2-yl}-1H-imidazol-2-yl)-1,3-benzothiazole,
5-{4-{1-[2-(methyloxy)ethyl]-4-(methylsulfonyl)-1H-benzimidazol-2-yl}-1H-imidazol-2-yl}-1,3-benzothiazole,
5-(4-{1-[2-(methyloxy)ethyl]-4-[(phenylmethyl)sulfonyl]-1H-benzimidazol-2-yl}-1H-imidazol-2-yl)-1,3-benzothiazole,
5-{4-{1-[2-(methyloxy)ethyl]-4-(4-morpholinyl)-1H-benzimidazol-2-yl}-1H-imidazol-2-yl}-1,3-benzothiazole,
2-[2-(1,3-benzothiazol-5-yl)-1H-imidazol-4-yl]-1-[2-(methyloxy)ethyl]-4-(4-morpholinyl)-1H-imidazo[4,5-c]pyridine,
2-[2-(1,3-benzothiazol-5-yl)-1H-imidazol-4-yl]-4-[(3S)-3-methyl-4-morpholinyl]-1-[2-(methyloxy)ethyl]-1H-imidazo[4,5-c]pyridine,
2-[2-(1,3-benzothiazol-5-yl)-1H-imidazol-4-yl]-4-[(3R)-3-methyl-4-morpholinyl]-1-[2-(methyloxy)ethyl]-1H-imidazo[4,5-c]pyridine,
2-[2-(1,3-benzothiazol-5-yl)-1H-imidazol-4-yl]-4-[(3R)-3-ethyl-4-morpholinyl]-1-[2-(methyloxy)ethyl]-1H-imidazo[4,5-c]pyridine,
2-[2-(1,3-benzothiazol-5-yl)-1H-imidazol-4-yl]-4-(2-methyl-4-morpholinyl)-1-[2-(methyloxy)ethyl]-1H-imidazo[4,5-c]pyridine,
((2S,5R)-4-{2-[2-(1,3-benzothiazol-5-yl)-1H-imidazol-4-yl]-1-[2-(methyloxy)ethyl]-1H-imidazo[4,5-c]pyridin-4-yl}-5-ethyl-2-morpholinyl)methanol,
((2S,5S)-4-{2-[2-(1,3-benzothiazol-5-yl)-1H-imidazol-4-yl]-1-[2-(methyloxy)ethyl]-1H-imidazo[4,5-c]pyridin-4-yl}-5-methyl-2-morpholinyl)methanol,
2-[2-(1,3-benzothiazol-5-yl)-1H-imidazol-4-yl]-1-[2-(methyloxy)ethyl]-4-(1-piperidinyl)-1H-imidazo[4,5-c]pyridine,
2-[2-(1,3-benzothiazol-5-yl)-1H-imidazol-4-yl]-1-[2-(methyloxy)ethyl]-4-(1-pyrrolidinyl)-1H-imidazo[4,5-c]pyridine,
4-(1-azetidinyl)-2-[2-(1,3-benzothiazol-5-yl)-1H-imidazol-4-yl]-1-[2-(methyloxy)ethyl]-1H-imidazo[4,5-c]pyridine,
2-[2-(1,3-benzothiazol-5-yl)-1H-imidazol-4-yl]-1-[2-(methyloxy)ethyl]-4-(4-methyl-1-piperazinyl)-1H-imidazo[4,5-c]pyridine,
2-[2-(1,3-benzothiazol-5-yl)-1H-imidazol-4-yl]-4-[(3S)-3,4-dimethyl-1-piperazinyl]-1-[2-(methyloxy)ethyl]-1H-imidazo[4,5-c]pyridine,
2-[2-(1,3-benzothiazol-5-yl)-1H-imidazol-4-yl]-N-methyl-N,1-bis[2-(methyloxy)ethyl]-1H-imidazo[4,5-c]pyridin-4-amine,
2-[{2-[2-(1,3-benzothiazol-5-yl)-1H-imidazol-4-yl]-1-[2-(methyloxy)ethyl]-1H-imidazo[4,5-c]pyridin-4-yl}(methyl)amino]ethanol,
2-[2-(1,3-benzothiazol-5-yl)-1H-imidazol-4-yl]-N-ethyl-N,1-bis[2-(methyloxy)ethyl]-1H-imidazo[4,5-c]pyridin-4-amine,
2-[2-(1,3-benzothiazol-5-yl)-1H-imidazol-4-yl]-6-methyl-1-[2-(methyloxy)ethyl]-4-(4-morpholinyl)-1H-imidazo[4,5-c]pyridine,
5-{4-[1-[2-(methyloxy)ethyl]-5-(4-morpholinylsulfonyl)-1H-benzimidazol-2-yl]-1H-imidazol-2-yl}-1,3-benzothiazole,
5-{4-[1-[2-(methyloxy)ethyl]-5-(1-piperazinylsulfonyl)-1H-benzimidazol-2-yl]-1H-imidazol-2-yl}-1,3-benzothiazole,
5-{4-[1-[2-(methyloxy)ethyl]-5-(1-pyrrolidinylsulfonyl)-1H-benzimidazol-2-yl]-1H-imidazol-2-yl}-1,3-benzothiazole,
2-[2-(1,3-benzothiazol-5-yl)-1H-imidazol-4-yl]-N,1-bis[2-(methyloxy)ethyl]-1H-benzimidazole-5-sulfonamide,
2-[2-(1,3-benzothiazol-5-yl)-1H-imidazol-4-yl]-6-methyl-N,1-bis[2-(methyloxy)ethyl]-1H-benzimidazole-5-sulfonamide,
2-[2-(1,3-benzothiazol-5-yl)-1H-imidazol-4-yl]-6-methyl-1-[2-(methyloxy)ethyl]-1H-benzimidazole-5-carboxylic acid,
5-(4-{6-methyl-1-[2-(methyloxy)ethyl]-5-[(4-methyl-1-piperazinyl)sulfonyl]-1H-benzimidazol-2-yl}-1H-imidazol-2-yl)-1,3-benzothiazole,
5-{4-[6-methyl-1-[2-(methyloxy)ethyl]-5-(4-morpholinylsulfonyl)-1H-benzimidazol-2-yl]-1H-imidazol-2-yl}-1,3-benzothiazole,
2-[2-(1,3-benzothiazol-5-yl)-1H-imidazol-4-yl]-1-[2-(methyloxy)ethyl]-1H-benzimidazole-5-carboxylic acid,
5-(1-methyl-4-{6-methyl-1-[2-(methyloxy)ethyl]-1H-benzimidazol-2-yl}-1H-imidazol-2-yl)-1,3-benzothiazole,
2′-(1,3-benzothiazol-5-yl)-1-[2-(methyloxy)ethyl]-1H,1′H-2,4′-biimidazole,
2′-(1,3-benzothiazol-5-yl)-4,5-dimethyl-1-[2-(methyloxy)ethyl]-1H,1′H-2,4′-biimidazole,
2′-(1,3-benzothiazol-5-yl)-4,5-diethyl-1-[2-(methyloxy)ethyl]-1H,1′H-2,4′-biimidazole, or
5-(4-{1-[2-(methyloxy)ethyl]-4,5,6,7-tetrahydro-1H-benzimidazol-2-yl}-1H-imidazol-2-yl)-1,3-benzothiazole,
or a pharmaceutically acceptable salt thereof.

50. A pharmaceutical composition comprising the compound according to claim 32, or a pharmaceutically acceptable salt thereof, and a pharmaceutically acceptable excipient.
